SEQUENCE LISTING (14 sequences)

SEQ ID NO: 1 - DNA, 414 bp, Artificial Sequence
  engineered cholera toxin subunit B (CTB) sequence
atgaaaaagacagctatcgcgattgcagtggcactggccggcttcgctaccgtagcgcaggccacacctcaaaatattactgatttgtgtgcagaataccacaacacacaaatacatacgctaaatgataagatattttcgtatacagaatctctagctggaaaaagagagatggctatcattacttttaagaatggtgcaacttttcaagtagaagtaccaggtagtgatagtaatataactcatatagattcacaaaaaaaagcgattgaaaggatgaaggataccctgaggattgcatatcttactgaagctaaagtcgaaaagttatgtgtatgggacaataataaaacgcctcatgcgattgccgcaattagtatggcaaatggatcccaccaccaccaccaccactag

SEQ ID NO: 2 - protein, 137 aa, Artificial Sequence
  translated cholera toxin subunit B (CTB) sequence
MKKTAIAIAVALAGFATVAQATPQNITDLCAEYHNTQIHTLNDKIFSYTESLAGKREMAIITFKNGATFQVEVPGSDSNITHIDSQKKAIERMKDTLRIAYLTEAKVEKLCVWDNNKTPHAIAAISMANGSHHHHHH

SEQ ID NO: 3 - DNA, 2016 bp, Artificial Sequence
  engineered exotoxin A of Pseudomonas aeruginosa (EPA)
atgaaaaagatttggctggcgctggctggtttagttttagcgtttagcgctagcgcgggcagcggcggtggcgatcagaacgcgaccggcagcggcggtggcaagcttgccgaggaagccttcgacctctggaacgaatgcgccaaggcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggtaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggctcaggcccagccgcgccgggaaaagcgctggagcgaatgggccagcggcaaggtgttgtgcctgctcgacccgctggacggggtctacaactacctcgcccagcagcgctgcaacctcgacgatacctgggaaggcaagatctaccgggtgctcgccggcaacccggcgaagcatgacctggacatcaaggataataataattctactcccacggtcatcagtcatcgcctgcatttccccgagggcggcagcctggccgcgctgaccgcgcaccaggcctgccacctgccgctggaggccttcactcgtcatcgccagccgcgcggctgggaacaactggagcagtgcggctatccggtgcagcggctggtcgccctctacctggcggcgcgactgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccagcgccgacgtggtgagcctgacctgccccgtcgccaaagatcaaaatagaactaaaggggaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcgcgcggccggatccgcaacggtgccctgctgcgggtctatgtgccgcgctggagtctgccgggcttctaccgcaccggcctgaccctggccgcgccggaggcggcgggcgaggtcgaacggctgatcggccatccgctgccgctgcgcctggacgccatcaccggccccgaggaggaaggcgggcgcgtgaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacttgaagcttggcagcggcggtggcgatcagaacgcgacctga

SEQ ID NO: 4 - protein, 671 aa, Artificial Sequence
  translated exotoxin A of Pseudomonas aeruginosa (EPA)
MKKIWLALAGLVLAFSASAGSGGGDQNATGSGGGKLAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAKDQNRTKGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKLGSGGGDQNAT

SEQ ID NO: 5 - DNA, 3027 bp, Artificial Sequence
  N. meningitidis PglA-I operon
atgaaaatcgtctttattaccaccgtcgcttcgagtatctatggcttccgtgcgccggttatcaaaaaactgattggcaaaaaccaccaggtctatgcgtttgtgagcgaattttctgataacgaactggacattatccgcgaaatgggcgtgaccccggttacgtaccgtagtaaccgctccggtctgaatccgttttcagatattaaatcgacctttctgatcttcaaagaactgaagaaaattagtccggatctggtttttccgtatttcgcgaaaccggtcatctttggcaccttcgcagcaaaactggcaggcgttccgcgcattgtcggtatgctggaaggcctgggttttgcattcaccccgcaaccggaaggcatcccgctgaaaacgaaaatcatcaaaggtattctgatcgcgctgtatcgtattgccctgccgatgctggaatcactgatcgttctgaacccggatgacaaagatgaactgaccgacaaatacggcatcaaaatcaaaaacatccatatcctgggcggtattggtctggatctgcgccagtatccgtactcggaagccgatattccggacgaaaaagaaccggtgaaatttctgttcatcggccgctttctgaaagaaaaaggtattgatgacttcatccgtgcagctgaacaagtgaaagataaatatccggacaccgtttttacggcgctgggtgccattgataaaagtcgtggcggtggcggtgacctggaacgtctggcagcccgcgatattatccgttttccgggcttcgtcaacaatgtgtccgaagttattaaagaacatcacatctttgtcctgccgagctattaccgcgaaggtgtgccgcgttctacccaggaagcaatggctgtgggccgcgcagttatcaccacggacgttccgggttgccgtgaaacggtcgctgataaagtgaacggctttctgattgaaccgtggaatccgcgtatcctggcagaaaaaatgatctacttcatcgaaaaccgcgaagctgtgcgtctgatgggtaatgcatcttacgctattgctaaagacaaattcgatgctgaaaaagtggatctgaaactgctggacattctgaaagcgtaatctagagtcgacaggaggaacattatgagccaagctctgccgtaccgcccggacattgataccctgcgtgccgctgctgtgctgagtgttattgtgttccatattgaaaaagattggctgccgggcggtttcctgggcgttgacatctttttcgtcatttccggttttctgatgaccacgattctgcatcgtgaaatgtctggcggtggcggtcgcttcagtctgaaagcattttatattcgtcgcatcaaacgtattctgccggcctttttcgcagtgctggcagcaaccctggcgggcggctttttcctgttcacgaaagatgactttttcctgctgtggaaatccgctctgaccgcgctgggcttcgcttcaaacctgtattttgcgcgcggtaaagattactttgacccggcccaggaagaaaaaccgctgctgcatatctggagcctgtctgtcgaagaacaattttatttcgtgtttccgattctgctgctgctggttgcgcgtaaatctctgcgcgtccagttcggctttctggcagctctgtgcgcactgagcctggcagcctcttttatcccgagtgcactggataaatattacctgccgcacctgcgtgcgtgtgaactgctgattggttctctgaccgctgtctggatgcgttgccgccagccggcggtgggccgtcgctgtgcagctgtgggtgccctgttcgcagtttgcattctgagtacctgtctgttcagttattccgaacaaacggcatactttccgggcccggcggcactgatcccgtgcctggcagtggcagctctgatttattttaaccattacgaacacccgctgaaaaaatttttccagtccaaaatcacggttgcggcgggtctgatttcatattcgctgtacctgtggcattggccgatcctggcgttcatgcgttatattggtccggataatctgccgccgtactcaccggcagctgcggtggttctgatcctgctgctgagtctgttttcctaccactgtatcgaaaaaccgttcaaaaaatggcagggctcctttgcccaatcagtgctgtggatctacgcactgccgatgctgattctgggcgctggtagctttttcgcgatgcgcctgccgtttatggcgcagtatgatcgtctgggtctgacccgcagcaacacgtcgtgccataacaataccggcaaacagtgtctgtggggtgatacggaaaaacaaccggaactgctggtgctgggcgattcgcatgccgaccactataaaacctttttcgatgcagttggtaaaaaagaaaaatggtcggctacgatggtcagcgcggacgcatgcgcgtatgtggaaggctacgcctctcgcgtttttcagaactgggccgcatgtcgtgccgtttatcgctacgcagaagaacatctgccgcgttattcgaaagtcgtgctggcgatgcgctggggcagccaaatgccggaaaatagccgttctctggcttacgatgcgggctttttccagaaatttgaccgcatgctgcacaaactgagcagcgaaaaacaagccgtgtatctgatggccgataacctggcaagttcctataatgttcagcgtgcgtacatcctgtcatcgcgcattccgggttaccgtcaagccctgcgcccggatgacgaaagtaccctgaaagctaacgcgcgtatccgcgaactggctgcgaaatatccgaatgtctacattatcgatgccgcagcttatatcccggcggactttcagattggcggtctgccggtttatagcgataaagaccacattaatccgtacggcggtacggaactggctaaacgcttctccgaaaaacaacgcttcctggacacccgtcataaccactga

SEQ ID NO: 6 - DNA, 1422 bp, Artificial Sequence
  N. meningitidis pglF (a flippase)
atggacacaaaagaaatcctcggctacgcggcaggctcgatcggcagcgcggttttagccgtcatcatcctgccgctgctgtcgtggtatttccccgccgacgacatcgggcgcatcgtgctgatgcagacggcggcgggactgactgtgtcggtattgtgcctcgggctggatcaggcatacgtccgcgaatactatgccgccgccgacaaagacaccttgttcaaaaccctgttcctgccgccgctgctgtctgccgccgcgatagccgccctgctgctttcccgcccgtccctgccgtctgaaatcctgttttcgctcgacgatgccgccgccggcatcgggctggtgctgtttgaactgagcttcctgcccatccgctttctcttactggttttgcgtatggaagggcgcgcccttgccttttcgtccgcgcaactcgtgtccaagctcgccatcctgctgctgctgccgctgacggtcgggctgctgcactttccggcgaacaccgccgtcctgaccgccgtttacgcgctggcaaaccttgccgccgccgcctttttgctgtttcaaaaccgatgccgtctgaaggccgtccggcgcgcaccgttttcatccgccgtcctgcatcgcggcctgcgctacggcataccgatcgcactaagcagcatcgcctattgggggctggcatccgccgaccgtttgttcctgaaaaaatatgccggcctagaacagctcggcgtttattcgatgggtatttcgttcggcggggcggcattattgttccaaagcatcttttcaacggtctggacaccgtatattttccgcgcaatcgaagcaaacaccccgcccgcccgcctctcggcaacggcagaatctgcctccgccctgcttgccgccgccgtctgcctgaccggcattttctcgcccctcgcctccctcctgctgccggaaaactacgccgccgtccggtttatcgtcgtatcgtgtatgctgccgccgctgttttgcacgctggtagaaatcagcggcatcggtttgaacgtcgtccgaaaaacacgcccgatcgcgctcgccaccttgggcgcgctggcggcaaacctgctgctgctggggcttgccgtaccgtccggcggcgcgcgcggcgcggcggttgcctgtgccgcctcattctggttgttttttgttttcaagacagaaagctcctgtcgcctgtggcagccgctcaaacgcctgccgctttatacgcacacattgctctgcctgacctcctcggcggcctacacctgcttcggcacaccggcaaactaccccctgtttgccggcgtatgggcggtatatctggcaggctgcatcctgcgccactggaaagatttgcacaaactgtttcattatttgaaaaaacaagggtttccattatga

SEQ ID NO: 7 - protein, 473 aa, Artificial Sequence
  translated N. meningitidis pglF (a flippase)
MDTKEILGYAAGSIGSAVLAVIILPLLSWYFPADDIGRIVLMQTAAGLTVSVLCLGLDQAYVREYYAAADKDTLFKTLFLPPLLSAAAIAALLLSRPSLPSEILFSLDDAAAGIGLVLFELSFLPIRFLLLVLRMEGRALAFSSAQLVSKLAILLLLPLTVGLLHFPANTAVLTAVYALANLAAAAFLLFQNRCRLKAVRRAPFSSAVLHRGLRYGIPIALSSIAYWGLASADRLFLKKYAGLEQLGVYSMGISFGGAALLFQSIFSTVWTPYIFRAIEANTPPARLSATAESASALLAAAVCLTGIFSPLASLLLPENYAAVRFIVVSCMLPPLFCTLVEISGIGLNVVRKTRPIALATLGALAANLLLLGLAVPSGGARGAAVACAASFWLFFVFKTESSCRLWQPLKRLPLYTHTLLCLTSSAAYTCFGTPANYPLFAGVWAVYLAGCILRHWKDLHKLFHYLKKQGFPL

SEQ ID NO: 8 - DNA, 9033 bp, Artificial Sequence
  synthetic N. meningitidis PglFBCDAI operon
atggacacaaaagaaatcctcggctacgcggcaggctcgatcggcagcgcggttttagccgtcatcatcctgccgctgctgtcgtggtatttccccgccgacgacatcgggcgcatcgtgctgatgcagacggcggcgggactgactgtgtcggtattgtgcctcgggctggatcaggcatacgtccgcgaatactatgccgccgccgacaaagacaccttgttcaaaaccctgttcctgccgccgctgctgtctgccgccgcgatagccgccctgctgctttcccgcccgtccctgccgtctgaaatcctgttttcgctcgacgatgccgccgccggcatcgggctggtgctgtttgaactgagcttcctgcccatccgctttctcttactggttttgcgtatggaagggcgcgcccttgccttttcgtccgcgcaactcgtgtccaagctcgccatcctgctgctgctgccgctgacggtcgggctgctgcactttccggcgaacaccgccgtcctgaccgccgtttacgcgctggcaaaccttgccgccgccgcctttttgctgtttcaaaaccgatgccgtctgaaggccgtccggcgcgcaccgttttcatccgccgtcctgcatcgcggcctgcgctacggcataccgatcgcactaagcagcatcgcctattgggggctggcatccgccgaccgtttgttcctgaaaaaatatgccggcctagaacagctcggcgtttattcgatgggtatttcgttcggcggggcggcattattgttccaaagcatcttttcaacggtctggacaccgtatattttccgcgcaatcgaagcaaacaccccgcccgcccgcctctcggcaacggcagaatctgcctccgccctgcttgccgccgccgtctgcctgaccggcattttctcgcccctcgcctccctcctgctgccggaaaactacgccgccgtccggtttatcgtcgtatcgtgtatgctgccgccgctgttttgcacgctggtagaaatcagcggcatcggtttgaacgtcgtccgaaaaacacgcccgatcgcgctcgccaccttgggcgcgctggcggcaaacctgctgctgctggggcttgccgtaccgtccggcggcgcgcgcggcgcggcggttgcctgtgccgcctcattctggttgttttttgttttcaagacagaaagctcctgtcgcctgtggcagccgctcaaacgcctgccgctttatacgcacacattgctctgcctgacctcctcggcggcctacacctgcttcggcacaccggcaaactaccccctgtttgccggcgtatgggcggtatatctggcaggctgcatcctgcgccactggaaagatttgcacaaactgtttcattatttgaaaaaacaaggtttcccattggattacaaggatgacgacgataagtgaactagtaggagggcaaatgagtaaattcttcaaacgcctgtttgacattgttgcctccgcctcgggactgattttcctctcgccagtatttttgattttgatatacctcatccgcaagaatctaggttcgcccgtcttcttctttcaggaacgccccggaaaggacggaaaaccttttaaaatggtcaaattccgttccatgcgcgacgcgcttgattcagacggcattccgctgcccgacggagaacgcctgacaccgttcggcaaaaaactgcgtgccgccagtttggacgaactgcctgaattatggaatatcttaaaaggcgagatgagcctggtcggcccccgcccgctgctgatgcaatatctgccgctgtacgacaacttccaaaaccgccgccacgaaatgaaacccggcattaccggctgggcgcaggtcaacgggcgcaacgcgctttcgtgggacgaaaaattcgcctgcgatgtttggtatatcgaccacttcagcctgtgcctcgacatcaaaatcctactgctgacggttaaaaaagtattaatcaaggaagggatttccgcacagggcgaagccaccatgccccctttcacaggaaaacgcaaactcgccgtcgtcggtgcgggcggacacggaaaagtcgttgccgaccttgccgccgcactcggccggtacagggaaatcgtttttctggacgaccgcgcacaaggcagcgtcaacggcttttccgtcatcggcacgacgctgctgcttgaaaacagtttatcgcccgaacaatacgacgtcgccgtcgccgtcggcaacaaccgcatccgccgccaaatcgccgaaaaagccgccgcgctcggcttcgccctgcccgttctggttcatccggacgcgaccgtctcgccttctgcaacagtcggacaaggcagcgtcgttatggcgaaagccgtcgtacaggcaggcagcgtattgaaagacggcgtgattgtgaacactgccgccaccgtcgatcacgactgcctgcttaacgctttcgtccacatcagcccaggcgcgcacctgtcgggcaacacgcatatcggcgaagaaagctggataggcacgggcgcgtgcagccgccagcagatccgtatcggcagccgcgcaaccattggagcgggcgcagtcgtcgtacgcgacgtttcagacggcatgaccgtcgcgggcaatccggcaaagccgctgccgcgcaaaaaccccgagacctcgacagcataagcgattaaaatacacccccgtacagaccgattttgacaacacctgcggcgcgcgcccgattcttcggaacacgccccccttcagacggcatagggtcggaaatgccgtctgaaaaccgacggacaaaccatcatgctgaacactttcctttccccgtggccctgcttcacccaagaagaagccgatgccgtttccaaagtcctgctgtccaacaaagtcaactactggacgggcaacgaatgccgcgaatttgaaaaagaatttgccgcctttgccggcacgcggtacgccgtcgcccttgccaacggcacgctggcactcgatgtcgcgctcaaagcaatgggcataggcgcgggcgacgatgtgattgttacctcgcgcaccttcctcgcttccgcgtcctgcattgtgaacgcgggcgcaaaccccgtgtttgccgatgtggatttgaacagccaaaacatcagcgcggaaaccgtcaaagccgcgctgacaccgactaccaaagccgtcatcgtcgtccacctcgccggtatgcccgccgaaatggacggcattatggctttggcaaaagaacataatctttgggtaatcgaagactgcgcccaagcgcacggcgcaaaatacaaaggcaaatccgtcggctctatcggacacgtcggcgcgtggtcgttctgccaagacaaaatcatgaccaccggcggcgaaggcggtatggttacgaccaacgacaaaaccctgtgggaaaaaatgtggtcgtacaaagaccacggcaaaagctacgatgccgtgtacaaccacgaacacgcgcccggtttccgctggctgcacgaaagtttcggcacaaactggcgtatgatggaaatgcaggcggtcatcggacgcatccagctcaaacgcctgcccgaatggacggcgcgccgccgagaaaacgccgccaagctggcggaaagtttgggcaaattcagcagcatccgcttggttgaagtcgccgactacatcggacacgcgcaatataagttctacgccttcgtcaaacccgaacacctcaaagacggctggacgcgcgaccgcatcgtcggcgaactgaacgcgcgcaaagtcccctgctatcaaggcagctgctccgaagtctatttggaaaaagccttcgacaacacgccgtggcgaccgaaagagcgtttgacaaatgctgtcgagttgggcgacaccagcctgatgttcttggtgcacccgacgctgaccgacgacgaaattgcgttttgcaaaaaacacatcgaagccgtcttgaccgaagccgcacgataacccttcagacggcatatgccgcctgaaaacacataccgccccacgatatgaatctggaaactctgatcgccctgccgcgcaacatcaagaaaatctgtttcctcatacacgattttctgatgattttcattgccttttggttcacccaaagcctaaaggccgactactcggacgaatggttcgattttgccaactggcagtcttttttgctgactgccttgctgaccatcacattatttatccgaatggggctttaccacgccgttacacgcttcgtcagcttccgcatcctcaccaccgcactggcgggcagcctcgcctccgccgtgttgtttttcctcaatacgctgatatttgaagaaaggctgcgcctcgccctgccgattgtctatttcttactgctgtttgtttccgtgaccggctcgcgtatggttttgcgcggactgttgtccgaacaccccaaaaaacagatgatccctgtcatcatttacggcgcgggacggtcgggcagacaactgcttgaggccgtcaaacaaatgcgcgaatattccgccgccgcctttgtagacgacgaccccaaactgtggcacaccgtcatctacgaccttgccgtttaccagcccgatgccatcgccttcctcatcgaacgctacggcgtggaaaaaatcctgctcgccatccccggcgcgacccaggaacaacgccgccgaatcatcaacaaactggaagcctatccgtgcgaagtgttgaccattcccggaatgaaagacctgatggacggaaaaatcagcatcggcacgctcaaaaaaatctctgtgtccgacctgctcgggcgtgattccgtcgcgcccgacgaccgcctgatgagtgccgacatcgaaggcaaaaccgtcatggtaaccggcgcgggcggctccatcggttcggaactctgccgccagattatccgccgccgccccgaaaagctgctgctgttcgagttatccgaattcgccctgtacgccatcgaaaaagaattgcgcgaaacctgcatccaaaaacgcctcgacaccgaaatcctgccctttctcggttcggtgcaaaaccgcacgctgctcgaacacgtcatgaccgccttttccgttgcgaccgtctatcacgccgctgcctacaaacacgtccccatggtcgagttcaacaccgtcgaaggcatacgcaacaacatcttcggcacactcgagtgcgcgcttgccgccacgacatcgggcgtaagaactttcgtcctcatctccaccgacaaagccgtccgccccaccaacaccatgggtgccagcaaacgcatggcggaactctgccttcaggcactcgccgccgaacccggacaaaaaacccgcttcagcatggtacgtttcggcaatgttttaggttcgtccggctccgttgtcccgctgtttgaaaaacagattgcagaaggcggcccgcttaccctgacccaccccgaaatcacacgttatttcatgaccatacccgaagccgcccaactcgtcatacaggcaggcgcgatgggtacgggcggcgacgtattcgtcctcgacatgggtgaatccgtcaaaatcatcgaccttgcccgccaaatgattaccctaagcggcctcaaacccaacacacccgaacaacccgacggcgacatcgaaatcctcattaccggactgcgtcccggagaaaaactctacgaagagctgctcatcggcgacaacgtccgcaaaaccggccatccgcgcatcatgaccgccaacgagaccatgctgccgtggcacgagctctccgccctgctcgaccgcatccgtgcggcctgcgaccgttacgaccagcaggcaatccgcaccctgctcatcaacgccccgaccggctttgccccgagcgacggcatctgcgacctgctttgggtacgagaaacacacagaaaaaatgccgtctgaaccttcagacccggggatccaggaggtgtccgccttatgaaaatcgtctttattaccaccgtcgcttcgagtatctatggcttccgtgcgccggttatcaaaaaactgattggcaaaaaccaccaggtctatgcgtttgtgagcgaattttctgataacgaactggacattatccgcgaaatgggcgtgaccccggttacgtaccgtagtaaccgctccggtctgaatccgttttcagatattaaatcgacctttctgatcttcaaagaactgaagaaaattagtccggatctggtttttccgtatttcgcgaaaccggtcatctttggcaccttcgcagcaaaactggcaggcgttccgcgcattgtcggtatgctggaaggcctgggttttgcattcaccccgcaaccggaaggcatcccgctgaaaacgaaaatcatcaaaggtattctgatcgcgctgtatcgtattgccctgccgatgctggaatcactgatcgttctgaacccggatgacaaagatgaactgaccgacaaatacggcatcaaaatcaaaaacatccatatcctgggcggtattggtctggatctgcgccagtatccgtactcggaagccgatattccggacgaaaaagaaccggtgaaatttctgttcatcggccgctttctgaaagaaaaaggtattgatgacttcatccgtgcagctgaacaagtgaaagataaatatccggacaccgtttttacggcgctgggtgccattgataaaagtcgtggcggtggcggtgacctggaacgtctggcagcccgcgatattatccgttttccgggcttcgtcaacaatgtgtccgaagttattaaagaacatcacatctttgtcctgccgagctattaccgcgaaggtgtgccgcgttctacccaggaagcaatggctgtgggccgcgcagttatcaccacggacgttccgggttgccgtgaaacggtcgctgataaagtgaacggctttctgattgaaccgtggaatccgcgtatcctggcagaaaaaatgatctacttcatcgaaaaccgcgaagctgtgcgtctgatgggtaatgcatcttacgctattgctaaagacaaattcgatgctgaaaaagtggatctgaaactgctggacattctgaaagcgtaatctagagtcgacaggaggaacattatgagccaagctctgccgtaccgcccggacattgataccctgcgtgccgctgctgtgctgagtgttattgtgttccatattgaaaaagattggctgccgggcggtttcctgggcgttgacatctttttcgtcatttccggttttctgatgaccacgattctgcatcgtgaaatgtctggcggtggcggtcgcttcagtctgaaagcattttatattcgtcgcatcaaacgtattctgccggcctttttcgcagtgctggcagcaaccctggcgggcggctttttcctgttcacgaaagatgactttttcctgctgtggaaatccgctctgaccgcgctgggcttcgcttcaaacctgtattttgcgcgcggtaaagattactttgacccggcccaggaagaaaaaccgctgctgcatatctggagcctgtctgtcgaagaacaattttatttcgtgtttccgattctgctgctgctggttgcgcgtaaatctctgcgcgtccagttcggctttctggcagctctgtgcgcactgagcctggcagcctcttttatcccgagtgcactggataaatattacctgccgcacctgcgtgcgtgtgaactgctgattggttctctgaccgctgtctggatgcgttgccgccagccggcggtgggccgtcgctgtgcagctgtgggtgccctgttcgcagtttgcattctgagtacctgtctgttcagttattccgaacaaacggcatactttccgggcccggcggcactgatcccgtgcctggcagtggcagctctgatttattttaaccattacgaacacccgctgaaaaaatttttccagtccaaaatcacggttgcggcgggtctgatttcatattcgctgtacctgtggcattggccgatcctggcgttcatgcgttatattggtccggataatctgccgccgtactcaccggcagctgcggtggttctgatcctgctgctgagtctgttttcctaccactgtatcgaaaaaccgttcaaaaaatggcagggctcctttgcccaatcagtgctgtggatctacgcactgccgatgctgattctgggcgctggtagctttttcgcgatgcgcctgccgtttatggcgcagtatgatcgtctgggtctgacccgcagcaacacgtcgtgccataacaataccggcaaacagtgtctgtggggtgatacggaaaaacaaccggaactgctggtgctgggcgattcgcatgccgaccactataaaacctttttcgatgcagttggtaaaaaagaaaaatggtcggctacgatggtcagcgcggacgcatgcgcgtatgtggaaggctacgcctctcgcgtttttcagaactgggccgcatgtcgtgccgtttatcgctacgcagaagaacatctgccgcgttattcgaaagtcgtgctggcgatgcgctggggcagccaaatgccggaaaatagccgttctctggcttacgatgcgggctttttccagaaatttgaccgcatgctgcacaaactgagcagcgaaaaacaagccgtgtatctgatggccgataacctggcaagttcctataatgttcagcgtgcgtacatcctgtcatcgcgcattccgggttaccgtcaagccctgcgcccggatgacgaaagtaccctgaaagctaacgcgcgtatccgcgaactggctgcgaaatatccgaatgtctacattatcgatgccgcagcttatatcccggcggactttcagattggcggtctgccggtttatagcgataaagaccacattaatccgtacggcggtacggaactggctaaacgcttctccgaaaaacaacgcttcctggacacccgtcataaccactga

SEQ ID NO: 9 - DNA, 10368 bp, Artificial Sequence
  synthetic N. meningitidis PglFB2CDAI operon
atggacacaaaagaaatcctcggctacgcggcaggctcgatcggcagcgcggttttagccgtcatcatcctgccgctgctgtcgtggtatttccccgccgacgacatcgggcgcatcgtgctgatgcagacggcggcgggactgactgtgtcggtattgtgcctcgggctggatcaggcatacgtccgcgaatactatgccgccgccgacaaagacaccttgttcaaaaccctgttcctgccgccgctgctgtctgccgccgcgatagccgccctgctgctttcccgcccgtccctgccgtctgaaatcctgttttcgctcgacgatgccgccgccggcatcgggctggtgctgtttgaactgagcttcctgcccatccgctttctcttactggttttgcgtatggaagggcgcgcccttgccttttcgtccgcgcaactcgtgtccaagctcgccatcctgctgctgctgccgctgacggtcgggctgctgcactttccggcgaacaccgccgtcctgaccgccgtttacgcgctggcaaaccttgccgccgccgcctttttgctgtttcaaaaccgatgccgtctgaaggccgtccggcgcgcaccgttttcatccgccgtcctgcatcgcggcctgcgctacggcataccgatcgcactaagcagcatcgcctattgggggctggcatccgccgaccgtttgttcctgaaaaaatatgccggcctagaacagctcggcgtttattcgatgggtatttcgttcggcggggcggcattattgttccaaagcatcttttcaacggtctggacaccgtatattttccgcgcaatcgaagcaaacaccccgcccgcccgcctctcggcaacggcagaatctgcctccgccctgcttgccgccgccgtctgcctgaccggcattttctcgcccctcgcctccctcctgctgccggaaaactacgccgccgtccggtttatcgtcgtatcgtgtatgctgccgccgctgttttgcacgctggtagaaatcagcggcatcggtttgaacgtcgtccgaaaaacacgcccgatcgcgctcgccaccttgggcgcgctggcggcaaacctgctgctgctggggcttgccgtaccgtccggcggcgcgcgcggcgcggcggttgcctgtgccgcctcattctggttgttttttgttttcaagacagaaagctcctgtcgcctgtggcagccgctcaaacgcctgccgctttatacgcacacattgctctgcctgacctcctcggcggcctacacctgcttcggcacaccggcaaactaccccctgtttgccggcgtatgggcggtatatctggcaggctgcatcctgcgccactggaaagatttgcacaaactgtttcattatttgaaaaaacaaggtttcccattggattacaaggatgacgacgataagtgaactagtgcgatcgcatgaataaatttttcaaacgcctgttcgacatcatcgcctccgcatcggggctgattgtcctctcacccgttttcctgattttggcgtacctcatccgcaagaatctaggttcgcccgtcttcttcattcaagaacgccccggaaaggacggaaaaccttttaaaatggtcaaattccgttccatgcgcgacgcgcttgattcagacggcattccgctgcccgacagcgagcggctcacacccttcggcaaaaaactgcgtgccgctagtttggacgaactgcctgaattatggaatgtcttaaaaggcgacatgagcctggtcggcccgcgcccgcttttgacgggctacctgccgctttataacgacttccaaaaccgccgccacgaaatgaaaccgggcattaccggctgggcgcaggtcaacgggcgcaacgcgctttcgtgggacgaaaagttcgcctgcgatgtttggtacaccgacaacttcagcttttggctggatatgaaaatcctgtttttgacagtcaaaaaagtcttgatcaaagaaggcatttcggcggaaggggaagccactatgccttatttcactggaaatcaaactaatgaaaaaaataatatactgattctgtcggcaggtcgccgtgtcgaattggtgcaggacttccaaaccgaagccgcacgtttttcagacggcatcagcgtttttgccactgacctcaacccccgcatgtcttctgcctgccatgtttcggacggttcatttgaagtgccgccgattagtgccgattcatatatcgacagcattttcaatctggcggtacgcgaaaatatcggtttaatcatcccaaccatagacaccgaactgcaaaagctggcagacgagcgcgaacgctttgaagcggcaagtgtccacatcgtcgtttccgattcggacttcatcgcacaatgccgcgacaaacggaaaaccgccaccttgtttgcccgctacggcatacgctcacccgaaatttacgaccgcaagaagttggttttcccttgcttcgccaagccttatgacggcagccgcgccatcggtgcaaaacggattgacacacctgccgacctgacaccggaagtcttggcagacaccaaattaatgttctgtcagtttatcgacatcgaaaataaattttccgagtttaccgtcgatatgtattacgaccgtcaatgccgtctgaaatgcgccatcccgcgaaaacgcttggaagtgcgctccggcgaaatcagcaaaggcgttacctgcaaaaacagcctctaccaaacgctgctggaaaaaatgtcggtattgcagggcgcgcgcggctgtatcacggcgcagttcttctacaacgaagaaaccggcgaattctacggcgtggaaatcaacccccgcttcggcggtggcttcccgctgacgtatgccgcaggcggcaattatccgagccggttgatgcgcgaatacctcggcgacgaagaaatcccgttttcagacgactgggaaaacaacctcattatgttgcgctacgatgccaaggtcttggttcatgaaaacgattaatcctgaaaccaccgtcatcgtcttcgacttggacgataccctgtattcggaatacgaatacaagctctccggtatccgttcggtcatcgataccgttgccgcactgtatcccgattggaattcagacaacctatggcgcaacatcgaccccgacggcaaagattggctggacaaattgtgccgccactgtggttttaatgaatcggaaaaacaggttcttttgtggcaataccggctgcaccgcccgacgctgactccgtatgctccgccggatttcctgtccgaactgaccgcgcccttcgccgcacgcgcgctgattaccgacggcagaagcctgacccaacgcctgaagctggaagccttgggtctgtcttccctgtttgacgacatcctcatctctgaagcctgctcttcggaaaagcccgacggcaaacgcttccgccatcttcaagacaaatatgccgacaaggcaggctgcttcatctatatcggcgacaacatttccaaagactttatcgcgccgaacactttgggctggataaccataggattgcttccctccggacagaacatccaccgccaccagcctgaaagttttaacacagaatatcatcccgacttttgggttaactcccttcaagatttagttcctataattgattcaaatacttaaccattacataatcaaacatgcactcacaatattaaaaggactatttcatgacaaaatattattcatggggcgtaaacggctttccgccctacgttttcgacaccatttggaaacgatggaagttgaatgggcgcgggggcggttatcgtgtgcgacatcccggacggcatgaccgtcgcgggcaatccggcaaagccgctgccgcgcaaaaaccccaagaccgggacggcataagcgattaaaatatagtggattaaatttaaatcaggacaaggcgacgaagccgcagacagtacagatagtacggcaaggcgaggcaacgccgtactggtttaaatttaatccactatgcacccccgtacagaccgattttgacaacacctgcggcgcgcgcccgattcctcgaaacacgcccccttcagacggcatagggtcggaaatgccgtctgaacccgacggacaaaccatcatgttgaacacttccctttccccgtggccctgcttcacccaagaagaagccgatgccgtttccaaagtcctcttgtccaacaaagtcaattactggacgggcagcgaatgccgcgaatttgaaaaagaatttgccgcctttgccggcacgcggtacgccgtcgccctttccaacggcacgctggcactcgatgccgcgctcaaagcaatcggcataggcgagggcgatgatgtggttgttacctcgcgcaccttcctcgcctccgcgtcctgcatcgtgaacgcgggcgcaaaccccgtgtttgccgacgtggatttgaacagccaaaacatcagcgcggaaaccgtcaaggctgtgctgaccccgaataccaaagcgattatcgtggttcaccttgccggtatgcctgccgaaatggacgacatcatggctttggcaaaagaacatgatttgtgggtaattgaagactgcgcccaagcgcacggcgcaaaatacaaaggcaaatccgtcggctctatcggacacgtcggcgcgtggtcgttctgccaagacaaaatcatgaccaccggcggcgaaggcggtatggttacgaccaacgacaaaaccctgtgggaaaaaatgtggtcgtacaaagaccacggcaaaagctacgatgccgtgtaccaccgcgaacacgcgcccggtttccgctggctgcacgaaagtttcggcacaaactggcgtatgatggaaatgcaggcggcaatcgggcgcatccagctcaaacgcctgcccgaatggactgcctgccgtcaagcgcacgccgccaagctggcggaaagcctaggcaaattcaaaagtatccgcttggttgaagtcgccgactacatcggacacgcgcaatataagttctacgctttcgtcaaacccgaacacctcaaagacggctggacgcgcgaccgcatcgtcagcgaactgaacgcgcgcaaagtcccctgctatcaaggcagctgctccgaagtctatttggaaaaatctttcgacgacacgccttggcggccgaaagagcgtttgaaaaatgctgtcgaactgggcgacaccagcctgatgttcttggtgcacccgacgctgaccgacgacgaaatcgcgttctgcaaagaacacatcgaagccgtattggtagaagccacacgataacccttcagacggcatatgccgcctgaaagcacataccgccccacgatatgactctggaaaccctgatcgccctgccgcgcaacatcaagaaaatctgtttcctcatacacgattttctgatgattttcattgcattctggttcacccaaagcctaaaggccgactactcgaacgaatggttcgaccccgccaactggcagtctttcctattgaccgcgtttttgaccgcctgcctgttcttcagaatggggctttaccgcgccgtcacacgcttcatcagcttccgcatcctctccaccgcgctggcaggcagcctcgcctccgccgtattgtttttcctcaatacgctgatatttgaagaacggctgcggctcgccctgccgattgtctatttcttactgctgtttgtttccgtgaccggctcgcgtatggttttgcgcggactgttgtccgaacaccccaaaaaacagatggcccccgtcattatttacggcgcagggcaggcgggcagacagcttctcgaggccgtcaaacaaatgcgcgaatattccgccgccgcctttgtggacgacgaccccaaactgtggcgcaccgtcatctgcggccttgccgtttaccgccccgacgacatcgccgccctcatcggacgctacggcgtggaaaaaatcctgctcgccattcccagcgcgactcaggaacaacgccgccgaatcatcaacaaactggaagcctatccgtgcgaagtgttgaccattcccggaatgaaagacctgatggacgggaaaatcagcatcggcacgctcaaaaaaatctctgtgtccgacctgctcgggcgcgattccgtcacacccgacgaccgcctgatgagtgccgacatcgaaggtaaaaccgtcatggtaaccggcgcgggcggctccatcggttcggaactctgccgccagattatccgccgccgccccgaaaagctgctgctgttcgagttatccgaattcgccctgtacgccgtcgaaaaagaattgcgcgaaacctgcatccaaaaacgcctcgacaccgaaatcctgccctttctcggttcggtgcaaaaccgcacgctgctcgaacacgtcatgaccgccttttccgttgcgaccgtctatcacgccgccgcctacaaacacgtccccatggtcgagttcaacaccgtcgaaggcatacgcaacaacatcttcggcacactcgaatgcgcgcttgccgccacggcttcgggcgtggaaaccttcgtcctcatctccaccgacaaagccgtccgccccaccaacacgatgggcgccagcaaacgtatggcggaactctgcctacaggcgctcgccgccgaacccggacaaaaaacccgcttcagcatggtgcgtttcggcaatgttttaggctcgtccggctccgtcgtcccgctgtttgaaaaacagattgcagaaggcggcccgcttaccctgacccaccccgacatcacacgttatttcatgaccatacccgaagccgcccaactcgtcatacaggcaggcgcgatgggtactggcggcgacgtattcgtcctcgatatgggcgaatccgtcaaaatcatcgaccttgcccgccaaatgattaccttaagcggcctcaaacccaaaacacccgaacaacccgacggcgacatcgaaatcctcattaccggactgcgtcccggcgaaaaactctacgaagagctgctcatcggcgacaacgtccgcaaaaccggccatccgcgcattatgacggctgacgaggcgatgctgccgtggcacgagctctctgtcctgctcgaccgcatccgtgcggcctgcgaccgttacgaccagcaggcaatccgcaccctgctcatcaacgccccgaccggctttgccccgagcgacggtatctgcgacctgctttgggtacgagaaacacacagaaaaaatgccgtctgacccggggatccaggaggtgtccgccttatgaaaatcgtctttattaccaccgtcgcttcgagtatctatggcttccgtgcgccggttatcaaaaaactgattggcaaaaaccaccaggtctatgcgtttgtgagcgaattttctgataacgaactggacattatccgcgaaatgggcgtgaccccggttacgtaccgtagtaaccgctccggtctgaatccgttttcagatattaaatcgacctttctgatcttcaaagaactgaagaaaattagtccggatctggtttttccgtatttcgcgaaaccggtcatctttggcaccttcgcagcaaaactggcaggcgttccgcgcattgtcggtatgctggaaggcctgggttttgcattcaccccgcaaccggaaggcatcccgctgaaaacgaaaatcatcaaaggtattctgatcgcgctgtatcgtattgccctgccgatgctggaatcactgatcgttctgaacccggatgacaaagatgaactgaccgacaaatacggcatcaaaatcaaaaacatccatatcctgggcggtattggtctggatctgcgccagtatccgtactcggaagccgatattccggacgaaaaagaaccggtgaaatttctgttcatcggccgctttctgaaagaaaaaggtattgatgacttcatccgtgcagctgaacaagtgaaagataaatatccggacaccgtttttacggcgctgggtgccattgataaaagtcgtggcggtggcggtgacctggaacgtctggcagcccgcgatattatccgttttccgggcttcgtcaacaatgtgtccgaagttattaaagaacatcacatctttgtcctgccgagctattaccgcgaaggtgtgccgcgttctacccaggaagcaatggctgtgggccgcgcagttatcaccacggacgttccgggttgccgtgaaacggtcgctgataaagtgaacggctttctgattgaaccgtggaatccgcgtatcctggcagaaaaaatgatctacttcatcgaaaaccgcgaagctgtgcgtctgatgggtaatgcatcttacgctattgctaaagacaaattcgatgctgaaaaagtggatctgaaactgctggacattctgaaagcgtaatctagagtcgacaggaggaacattatgagccaagctctgccgtaccgcccggacattgataccctgcgtgccgctgctgtgctgagtgttattgtgttccatattgaaaaagattggctgccgggcggtttcctgggcgttgacatctttttcgtcatttccggttttctgatgaccacgattctgcatcgtgaaatgtctggcggtggcggtcgcttcagtctgaaagcattttatattcgtcgcatcaaacgtattctgccggcctttttcgcagtgctggcagcaaccctggcgggcggctttttcctgttcacgaaagatgactttttcctgctgtggaaatccgctctgaccgcgctgggcttcgcttcaaacctgtattttgcgcgcggtaaagattactttgacccggcccaggaagaaaaaccgctgctgcatatctggagcctgtctgtcgaagaacaattttatttcgtgtttccgattctgctgctgctggttgcgcgtaaatctctgcgcgtccagttcggctttctggcagctctgtgcgcactgagcctggcagcctcttttatcccgagtgcactggataaatattacctgccgcacctgcgtgcgtgtgaactgctgattggttctctgaccgctgtctggatgcgttgccgccagccggcggtgggccgtcgctgtgcagctgtgggtgccctgttcgcagtttgcattctgagtacctgtctgttcagttattccgaacaaacggcatactttccgggcccggcggcactgatcccgtgcctggcagtggcagctctgatttattttaaccattacgaacacccgctgaaaaaatttttccagtccaaaatcacggttgcggcgggtctgatttcatattcgctgtacctgtggcattggccgatcctggcgttcatgcgttatattggtccggataatctgccgccgtactcaccggcagctgcggtggttctgatcctgctgctgagtctgttttcctaccactgtatcgaaaaaccgttcaaaaaatggcagggctcctttgcccaatcagtgctgtggatctacgcactgccgatgctgattctgggcgctggtagctttttcgcgatgcgcctgccgtttatggcgcagtatgatcgtctgggtctgacccgcagcaacacgtcgtgccataacaataccggcaaacagtgtctgtggggtgatacggaaaaacaaccggaactgctggtgctgggcgattcgcatgccgaccactataaaacctttttcgatgcagttggtaaaaaagaaaaatggtcggctacgatggtcagcgcggacgcatgcgcgtatgtggaaggctacgcctctcgcgtttttcagaactgggccgcatgtcgtgccgtttatcgctacgcagaagaacatctgccgcgttattcgaaagtcgtgctggcgatgcgctggggcagccaaatgccggaaaatagccgttctctggcttacgatgcgggctttttccagaaatttgaccgcatgctgcacaaactgagcagcgaaaaacaagccgtgtatctgatggccgataacctggcaagttcctataatgttcagcgtgcgtacatcctgtcatcgcgcattccgggttaccgtcaagccctgcgcccggatgacgaaagtaccctgaaagctaacgcgcgtatccgcgaactggctgcgaaatatccgaatgtctacattatcgatgccgcagcttatatcccggcggactttcagattggcggtctgccggtttatagcgataaagaccacattaatccgtacggcggtacggaactggctaaacgcttctccgaaaaacaacgcttcctggacacccgtcataaccactga

SEQ ID NO: 10 - protein, 23 aa, Artificial Sequence
  peptide 1 from tryptic digested glycosylated CTB
NGATFQVEVPGSDSNITHIDSQK

SEQ ID NO: 11 - protein, 24 aa, Artificial Sequence
  peptide 2 from tryptic digested glycosylated CTB
NGATFQVEVPGSDSNITHIDSQKK

SEQ ID NO: 12 - protein, 8 aa, Artificial Sequence
  peptide 3 from tryptic digested glycosylated CTB
LCVWDNNK

SEQ ID NO: 13 - protein, 19 aa, Artificial Sequence
  tryptic glycopeptides identified containing Gal(OAc)-DATDH
HDLDIKDNNNSTPTVISHR

SEQ ID NO: 14 - protein, 16 aa, Artificial Sequence
  tryptic glycopeptides identified containing Gal(OAc)-DATDH
GSGGGDQNATGSGGGK

(19) United States
(12) Patent Application Publication
(10) Pub. No.: US 2014/0336366 A1
(43) Pub. Date: Nov. 13, 2014

(54) BIOCONJUGATE VACCINES MADE IN PROKARYOTIC CELLS

(71) Applicant: Amirreza Faridmoayer, Zurich (CH)

(72) Inventor: Amirreza Faridmoayer, Zurich (CH)

(73) Assignee: GLYCOVAXYN AG, Schlieren (CH)

(21) Appl. No.: 14/342,958

(22) PCT Filed: Sep. 6, 2012
(86) PCT No.: PCT/EP2012/067460

§ 371 Date: Jun. 24, 2014

Related U.S. Application Data
(60) Provisional application No. 61/531,577, filed on Sep. 6, 2011.

(51) Int. Cl. C07K 14/22; C12P 21/00

(52) U.S. Cl. CPC C07K 14/22; C12P 21/005
USPC 530/395; 435/252.33; 435/69.1

(57) ABSTRACT

Provided herein are prokaryotic cells capable of producing bioconjugates comprising glycosylated proteins. Also provided herein are compositions comprising such bioconjugates and/or comprising the saccharide moieties of such bioconjugates, as well as methods of vaccinating subjects using such compositions.

### 1. INTRODUCTION

Provided herein are prokaryotic cells capable of producing bioconjugates comprising glycosylated proteins and methods of producing such prokaryotic cells. Also provided herein are compositions comprising such bioconjugates and/or comprising the saccharide moieties of such bioconjugates, as well as methods of vaccinating subjects using such compositions.

### 2. BACKGROUND

Glycosylation is a biological process that is observed in all domains of life. The process of glycosylation comprises the addition of carbohydrates to an acceptor molecule, such as a protein or a polypeptide chain, and is involved in many biological functions, including cellular interactions, protein folding, secretion, and degradation.

N-glycosylation, the addition of oligosaccharides to an asparagine residue of a protein, is the most frequent post-translational modification of eukaryotic organisms. N-glycosylation takes place in the endoplasmic reticulum, where a preassembled oligosaccharide is transferred from a lipid carrier (dolychol phosphate) to an asparagine residue of a nascent protein by the enzyme oligosaccharyltransferase (OST), within the conserved sequence Asn-X-Ser/Thr (where X is any amino acid except proline). The saccharidic chain is then subject to other modifications in the Golgi apparatus.

Previous studies have demonstrated how to generate E. coli strains that can perform N-glycosylation (see, e.g., Wacker et al., Science. 2002; 298(5599):1790-3; Nita-Lazar et al., Glycobiology. 2005; 15(4):361-7; Feldman et al., Proc Natl Acad Sci USA. 2005; 102(8):3016-21; Kowarik et al., EMBO J. 2006; 25(9):1957-66; Wacker et al., Proc Natl Acad Sci USA. 2006; 103(18):7088-93; International Patent Application Publication Nos. WO2003/074687, WO2006/119987, WO 2009/104074, and WO/2011/06261; and International Patent Application No. PCT/EP2011/057111). Another form of glycosylation, O-glycosylation, comprises the modification of serine or threonine residues. In addition, Campylobacter jejuni, a Gram-negative bacterium, possesses its own glycosylation machinery. This machinery is encoded by a cluster called pgl (for protein glycosylation), which can be transferred to E. coli to allow for the glycosylation of recombinant proteins expressed by the E. coli

Neisseria meningitidis is a leading cause of meningitis. It has been estimated by the World Health Organization that there are over one million cases of invasive meningococcal disease per year, with an average mortality rate of 9%. N. meningitidis bacteria are classified into several groups based on their cell surface glycans, such as capsular polysaccharide (CPS) and lipooligosaccharide (LOS). To date, several glycoconjugate-based meningococcal vaccines have been successfully developed by chemical coupling of CPS isolated from N. meningitidis to proteins; however, production of CPS-based vaccines has not been successful for N. meningitidis serogroup B, due to the fact that group B CPS is similar to the human proteoglycan in structure. Recent studies have identified novel oligosaccharides that are located on the surface of various Neisseria species, such as N. meningitidis, N. gonnorroae, and N. lactamica (see, e.g., Borud et al., 2010, J. Bacteriol. 192(11):2816-2829). These oligosaccharides are attached to a number of cell-surface proteins such as pilin via O-glycosidic linkages. The process that governs O-glycosylation in this organism is analogous to Campylobacter N-glycosylation pathway. Hence, these surface glycans represent new targets for vaccine development, particularly in the development of vaccines against serogroup B strains of N. meningitidis.

In this invention a method for production of original glycosylated proteins with different Neisserial surface oligosaccharides that can be used as vaccine candidates is developed. In order to produce novel and innovative glycosylated proteins, different E. coli strains were engineered combining for the first time Neisseria protein O-glycosylation and Campylobacter spp. protein N-glycosylation systems.

### 3. SUMMARY

The present invention provides methods for the glycosylation of a target protein with a monosaccharide, disaccharide, or a trisaccharide in a prokaryotic host. In certain embodiments, the glycosylation with the monosaccharide, disaccharide, or a trisaccharide occurs at an asparagine residue of a glycosylation consensus sequence, e.g., Asn-X-Ser/Thr, wherein X can be any amino acid except Pro; or an Asp/Glu-X-Asn-Z-Ser/Thr, wherein X and Z can be any amino acid except Pro. More specifically, in certain embodiments, the consensus sequence for N-glycosylation is introduced recombinantly into the target protein. In certain embodiments, an oligosaccharyltransferase is introduced into the prokaryotic host. The oligosaccharyltransferase can be from any source. In a specific embodiment, the oligosaccharyltransferase is from Campylobacter.

The present invention also provides prokaryotic host cells that have been engineered for the biosynthesis of specific conjugates based on methods described herein. The present invention further provides bioconjugates generated from engineered prokaryotes with the methods described above. In certain embodiments, the prokaryotic host cells provided herein are engineered to express a set of the genes required for biosynthesis of a lipid linked oligosaccharide including, but not limited to, oligosaccharyltransferase(s), flippase(s), and glycosyltransferase(s).

The present invention further provides culture conditions for the glycosylation of the target protein. In certain embodiments, MgCl₂ is added to the culture medium, in particular 1 to 100 mM MgCl₂, 1 to 50 mM MgCl₂, 1 to 25 mM MgCl₂, 1 to 10 mM MgCl₂, 5 to 100 mM MgCl₂, 5 to 50 mM MgCl₂, 5 to 25 mM MgCl₂, 5 to 15 mM MgCl₂, at least 1 mM MgCl₂, at least 5 mM MgCl₂, at least 10 mM MgCl₂, at least 15 mM MgCl₂, at least 20 mM MgCl₂, or at least 25 mM MgCl₂ is added. In specific embodiments, at most 1 mM MgCl₂, at most 5 mM MgCl2, at most 10 mM MgCl₂, at most 15 mM MgCl₂, at most 20 mM MgCl₂, or at most 25 mM MgCl₂ is added. In a specific embodiment, 10 mM MgCl₂ is added. In a specific embodiment, the MgCl₂ concentration in the culture medium is 10 mM.

In certain embodiments, Terrific Broth is used as culture medium for the prokaryotic host cells provided herein. In certain embodiments, multiple open reading frames are introduced on a single plasmid to reduce the number of different antibiotics required to select for the presence of the plasmid.

In one aspect, provided herein are bioconjugates, e.g., isolated bioconjugates, that comprise a carrier protein and an oligosaccharide.

In a specific embodiment, provided herein is a bioconjugate comprising a carrier protein and a monosaccharide. Those of skill in the art will recognize that based on the discovery of the instant invention, monosaccharides can be transferred to carrier proteins. Thus, those of skill in the art will recognize that the particular monosaccharides selected for use in accordance with the methods described herein are not limited. In certain embodiments, the monosaccharide is DATDH or GATDH. In another specific embodiment, provided herein is a bioconjugate comprising a carrier protein and a monosaccharide, wherein the monosaccharide is from N. meningitidis.

In another specific embodiment, provided herein is a bioconjugate comprising a carrier protein and a disaccharide. Those of skill in the art will recognize that based on the discovery of the instant invention, disaccharides can be transferred to carrier proteins. Thus, those of skill in the art will recognize that the particular disaccharides selected for use in accordance with the methods described herein are not limited. In certain embodiments, the disaccharide is Gal-DATDH, Gal(OAc)-DATDH, Gal-GATDH, Gal-GATDH, Gal(OAc)-GATDH, Gal-GlcNAc, Gal(OAc)-GlcNAc, Glc-DATDH, or Glc-GATDH. In another specific embodiment, provided herein is a bioconjugate comprising a carrier protein and a disaccharide, wherein the disaccharide is from N. meningitidis.

In another specific embodiment, provided herein is a bioconjugate comprising a carrier protein and a trisaccharide. Those of skill in the art will recognize that based on the discovery of the instant invention, trisaccharides can be transferred to carrier proteins. Thus, those of skill in the art will recognize that the particular trisaccharides selected for use in accordance with the methods described herein are not limited. In certain embodiments, the trisaccharide is Gal(OAc)-Gal-DATDH, Gal-Gal-DATDH, Gal(OAc)-Gal-GATDH, or Gal-Gal-GATDH. In another specific embodiment, provided herein is a bioconjugate comprising a carrier protein and a trisaccharide, wherein the trisaccharide is from N. meningitidis.

In certain embodiments, the monosaccharide, disaccharide, or trisaccharide of the bioconjugates provided herein is covalently bound to the Asn within a glycosylation site of the carrier protein, wherein the glycosylation site comprises the amino acid sequence Asp/Glu-X-Asn-Z-Ser/Thr wherein X and Z may be any amino acid except Pro. In certain embodiments, the carrier proteins of the bioconjugates provided herein do not naturally (e.g., in their normal/native, or “wild-type” state) comprise a glycosylation site. In certain embodiments, the carrier proteins of the bioconjugates provided herein are engineered to comprise one or more glycosylation sites, e.g., the carrier proteins are engineered to comprise one or more glycosylation sites comprising the amino acid sequence Asp/Glu-X-Asn-Z-Ser/Thr wherein X and Z may be any amino acid except Pro. For example, the carrier proteins used in accordance with the methods described herein may comprise 1, 2, 3, 4, 5, 6, 7, 8, 9, 10 or more glycosylation sites, each having the amino acid sequence Asp/Glu-X-Asn-Z-Ser/Thr, wherein X and Z may be any amino acid except Pro; and wherein some (e.g., 1, 2, 3, 4, 5, 6, 7, 8, or 9) or all of the glycosylation sites have been recombinantly introduced into the carrier protein.

Any carrier proteins suitable for use in the methods described herein can be used in accordance with the methods described herein. Exemplary carrier proteins include, without limitation, Exotoxin A of P. aeruginosa, CRM 197, Diphtheria toxoid, tetanus toxoid, detoxified hemolysin A of S. aureus, clumping factor A, clumping factor B, E. coli FimH, E. coli FimHC, E. coli heat labile enterotoxin, detoxified variants of E. coli heat labile enterotoxin, Cholera toxin B subunit, cholera toxin, detoxified variants of cholera toxin, E. coli sat protein, the passenger domain of E. coli sat protein, C. jejuni AcrA, and a C. jejuni natural glycoprotein.

In a specific embodiment, the carrier protein to generate a bioconjugate described herein is an antigen of Neisseria, e.g., an antigen of Neisseria meningitidis such as an antigen of N. meningitidis group B. Exemplary antigens of N. meningitidis include, without limitation, pilin, NMB0088, nitrite reductase (AniA), heparin-binding antigen (NHBA), factor H binding protein (fHBP), adhesion, NadA, Ag473, or surface protein A (NapA).

In a second aspect, provided herein are prokaryotic host cells capable of producing the bioconjugates described herein.

In a specific embodiment, provided herein is a prokaryotic host cell for generating a bioconjugate, wherein the prokaryotic host cell comprises: (i) a heterologous nucleotide sequence encoding a carrier protein comprising at least one glycosylation site comprising the amino acid sequence Asp/Glu-X-Asn-Z-Ser/Thr wherein X and Z may be any natural amino acid except Pro; and (ii) a heterologous nucleotide sequence encoding an oligosaccaryltransferase; wherein the prokaryotic host cell is recombinantly engineered to produce Und-PP-monosaccharide, Und-PP-disaccharide, or Und-PP-trisaccharide and wherein the oligosaccharyltransferase transfers the disaccharide or the trisaccharide to the Asn of the glycosylation site. In a specific embodiment, the prokaryotic host cells described herein are E. coli host cells. In another specific embodiment, the oligosaccaryltransferase recombinantly introduced into the host cells described herein, e.g., E. coli host cells, is PglB of Campylobacter jejuni.

In certain embodiments, the host cells described herein comprise heterologous nucleic acid sequences (i.e., nucleic acid sequences, e.g., genes, that are not normally associated with the host cell in its natural/native state, e.g., its “wild-type” state) in addition to heterologous oligo saccharyltransferases. Such additional heterologous nucleic acid sequences may comprise, without limitation, flippases (e.g., PglK of Campylobacter jejuni or PglF of Neisseria meningitidis); and/or glycosyltransferases (e.g., PglA of Neisseria meningitidis or RfpB of Shigella dysenteriae. In specific embodiments, the heterologous nucleic sequences recombinantly introduced into the host cells described herein (e.g., prokaryotic host cells, e.g., E. coli) include nucleic acids that encode the genes for N. meningitidis PglB, PglC, and PglD; N. meningitidis PglB2, PglC, and PglD; N. meningitidis PglB, PglC, and PglD, PglF, PglA and PglI; and/or N. meningitidis PglB2, PglC, and PglD, PglF, PglA and PglI. In yet other specific embodiments, the heterologous nucleic sequences recombinantly introduced into the host cells described herein (e.g., prokaryotic host cells, e.g., E. coli) include nucleic acids that encode the genes corresponding to the entire pgl cluster of Campylobacter jejuni, or the entire pgl cluster of Campylobacter jejuni carrying a mutation or deletion of a desired gene, e.g., a transposon mutation in galE of the cluster.

In certain embodiments, the prokaryotic host cells provided herein produce and flip Und-PP-DATDH, Und-PP-GATDH, Und-PP-DATDH-Gal(OAc), and/or Und-PP-GATDH-Gal(OAc).

In yet another aspect, provided herein are methods of generating the bioconjugates provided herein. In certain embodiments, the methods for generating the bioconjugates provided herein comprise culturing a host cell described herein under conditions suitable for the production of proteins, and isolating the bioconjugate. Those of skill in the art will recognize conditions suitable for the maintenance of growth of host cells such that the bioconjugates described herein can be produced by the host cells and subsequently isolated. Such methods are additionally encompassed by the working Examples provided herein (see Section 6).

In yet another aspect, provided herein are compositions, e.g., immunogenic compositions, comprising the bioconjugates described herein. In certain embodiments, the immunogenic compositions described herein comprise a bioconjugate described herein and one or more additional components, e.g., an adjuvant.

### 4. DESCRIPTION OF THE FIGURES

FIG. 1 depicts “plasmid p6,” used for expression of cholera toxin subunit B (CTB) with two N-glycosylation sites with C-terminal hexa-His tags. The map of plasmid p6 is shown in (A). An OmpA signal sequence has been used for expression of CTB in the periplasm. (B) shows the DNA and translated protein sequence of plasmid p6. Underlined Asn residues in the protein sequence show the location of N-glycosylation sites.

FIG. 2 depicts “plasmid p18,” used for expression of exotoxin A of Pseudomonas aeruginosa (EPA) with four N-glycosylation sites. The map of plasmid p18 is shown in (A). DsbA signal sequence has been used for expression of EPA in the periplasm. (B) shows the DNA and translated protein sequence of plasmid p18. Underlined Asn residues in the protein sequence show the location of N-glycosylation sites.

FIG. 3 depicts “plasmid p15,” used for expression of a synthetic N. meningitidis PglA-I operon. pglA encodes the Galactosyltransferase and pglI encodes the O-acetyltransferase of N. meningitidis. In this plasmid, a synthetic, codon usage optimized operon of PglA-I was cloned into the pMLBAD vector. The map of plasmid p15 is shown in (A). (B) shows the DNA sequence of the PglA-I operon.

FIG. 4 depicts “plasmid p13,” used for expression of N. meningitidis pglF, encoding a flippase. pglF was amplified by PCR from genomic DNA of N. meningitidis L2 35E and cloned into pMLBAD. The map of plasmid p13 is shown in (A). (B) shows the DNA and translated protein sequence of plasmid p13.

FIG. 5 depicts “plasmid p17,” used for expression of a synthetic N. meningitidis PglFBCDAI operon. A synthetic PglFBCDAI operon was cloned into pMLBAD. The map of plasmid p17 is shown in (A). (B) shows the DNA sequence of the PglFBCDAI operon. Plasmid p17 contains all the necessary genes required for biosynthesis of Gal(OAc)-DATDH-Undpp and translocation into the periplasm.

FIG. 6 depicts “plasmid p20,” used for expression of a synthetic N. meningitidis PglFB2CDAI operon. A synthetic PglFB2CDAI operon was cloned into pMLBAD. The map of plasmid p20 is shown in (A). (B) shows the DNA sequence of the PglF-B2-C-D-A-I operon. Plasmid p20 contains all the necessary genes required for biosynthesis of Gal(OAc)-GATDH-Undpp and translocation into the periplasm.

FIG. 7 demonstrates the synthesis of different lipooligosaccharide (LOS) structures in E. coli using the C. jejuni N-glycan biosynthetic pathway. E. coli SCM7 and E. coli Sφ874 ΔwecA-wecG (see, e.g., Alaimo et al., EMBO, 2006) were transformed with different C. jejuni 81116 pgl plasmids harboring transposon mutations in different genes of the biosynthetic pathway gene cluster (see, e.g., Linton et al., Mol. Micro., 2005). Whole-cell extracts were digested with proteinase K in Laemmli sample buffer, polyacrylamide gel electrophoresis (PAGE) was performed, and the gels subjected to silver staining. Lane 1, “plasmid p1” (pACYC184, empty vector); lane 2, “plasmid p3” (pACYCpglA::Kan); lane 3, “plasmid p4” (pACYCpglJ::Kan); lane 4, “plasmid p5” (pACYCpglH::Kan); lane 5, “plasmid p2” (pACYCpgl, containing the entire protein glycosylation cluster). All plasmids for synthesis of C. jejuni truncated oligosaccharides and N-glycan variants were under the control of a constitutive promoter (see Wacker et al., Science, 2002). Shake flask cultures were harvested after overnight incubation at 37° C. Digested Whole-cell extracts (0.01 OD) were run on 12% Bis-Tris NuPage gels (Invitrogen) using MES buffer for 50 min at 200V.

FIG. 8 demonstrates glycosylation of engineered cholera toxin subunit B (CTB) with truncated variants of C. jejuni N-glycan in E. coli. Western-blot analysis of the periplasmic extract of E. coli SCM6 and E. coli SCM7ΔwaaL, co-transformed with plasmid p6, encoding engineered CTB, containing two N-glycosylation sites, and two other plasmids for sugar production and PglB expression is depicted. Lane 1, pEXT21 empty vector (“plasmid p7”) and “plasmid p8” (pACYCpglBmut, containing C. jejuni whole protein glycosylation cluster with inactivated PglB by double mutation of W458A/D459A); lane 2, “plasmid p9” (expressing C. jejuni PglB) and plasmid p8; lane 3, plasmid p9 and plasmid p5; lane 4, plasmid p9 and plasmid p7. Shake flask cultures were inoculated with overnight preculture and kept at 37° C. in a shaker incubator until reaching OD of 0.4-0.8. Cultures were induced with 0.02% Arabinose and 1 mM IPTG and incubated overnight at 37 C in the shaker incubator. Amounts (equal to 0.02 OD) of the periplasmic extracts were loaded onto a NuPAGE 12% SDS-Gel (Invitrogen) and run with MES buffer at 200 V for 65 min and electro-blotted with iBlot. The blot was developed after blocking with 10% milk in PBST and incubated with anti-CTB polyclonal antibody (1:1000) followed by incubation with HRP coupled anti-rabbit (1:10,000, BioRad).

FIG. 9 demonstrates the functional characterization of N. meningitidis flippase, PglF. E. coli SCM7, lacking undecaprenylpyrophosphate linked glycan flippase activity, was transformed with “plasmid p10” (containing the C. jejuni 81116 pgl cluster with a transposon mutation in pglK, inactivating flippase of N-glycoslyation pathway) and complementation of flipping activity was tested with other flippases: lane 1, “plasmid p11” (pMLBAD, empty vector); lane 2, “plasmid p12”, expressing functional C. jejuni PglK (see Alaimo et al. EMBO, 2006); lane 3; plasmid p13 (pGVX654 expressing N. meningitidis 35E pglF). Cultures were grown overnight at 37° C. in a shake flask, then cells were harvested and digested with proteinase K in Laemmli buffer. Digested samples (0.01 OD) were run on a 12% Bis-Tris NuPage SDS-gel (Invitrogen) using MES buffer for 50 min at 200V.

FIG. 10 demonstrates functional characterization of N. meningitidis PglA, galactosyltransferase, in E. coli. A shows a silver-stained PAGE profile of LOS produced in E. coli SCM7 harboring: Lane 1, plasmid p2 (pACYCpgl whole protein glycosylation cluster) and an empty vector control (pMLBAD, plasmid p11); lane 2, plasmid p3 (contains C. jejuni pgl cluster with a transposon mutation of the pglA, which impairs assembly of the first GalNAc onto Undpp-DATDH) and an empty vector (pMLBAD); lane 3, plasmid p3 and plasmid p14 (expressing Shigella dysenteriae O1 rfpB, which encodes an O-antigen Gal-transferase that assembles Gal to Undpp-GlcNAc); lane 4, plasmid p3 and plasmid p15 (N. meningitidis pglA-I operon, which encodes a Gal-transferase and O-acetyl transferase, respectively); lane 5; empty vector controls pMLBAD (plasmid p11) and pACYC184 (plasmid p1). B illustrates results of Western-blot analysis of corresponding samples from panel A using antibody that specifically recognizes C. jejuni heptasaccharide (N-glycan). Cultures were inoculated after induction with 0.2% Arabinose and grown overnight at 37° C. in a shake flask, then cells were harvested and digested with proteinase K, followed by silver staining of amounts of sample (0.01 OD) as well as Western blotting of amounts of sample (0.01 OD). For both, samples were run on a 12% Bis-Tris NuPage SDS-gel (Invitrogen) using MES buffer for 50 min at 200V.

FIG. 11 demonstrates N. meningitidis PglA specificity. E. coli SCM3 (E. coli Sφ874ΔwaaL) was transformed with plasmid p15, encoding pglA and pglI, or empty vector (pMLBAD, plasmid p11), and lipid-linked oligosaccharide (LLO) was extracted and analyzed. (A) shows an HPLC chromatogram of LLO labeled with 2AB run resolved with GlycoSep™ N Column (Prozyme). The arrow indicates a strong peak that was observed in the chromatogram of the extracts from cells harboring plasmid p15 (solid line) compared to control extract from cells with empty vector, plasmid p11 (dashed line). (B) shows the MS/MS analysis of the above indicated peak.

FIG. 12 demonstrates in vitro analysis of the LLO extract from E. coli SCM3 expressing N. meningitidis pglA-I. Enriched LLO extracts (200 OD) comprising the same samples as used in FIG. 14, were used for an in vitro N-glycosylation assay using purified C. jejuni PglB and a synthetic peptide, Tamra-DANYTK. (A) illustrates PAGE analysis of the glycopeptide after in vitro glycosylation. In (B), an in vitro glycopeptide sample (the same sample as lane 3 in panel A) was treated with α1-3,6 galactosidase from Xanthamonas manihotis (NEB) and subjected to PAGE analysis. Lane 1, untreated glycopeptide; lane 2, treated glycopeptide with galactosidase; lane 3; equal amounts of treated and untreated sample mixed prior loading to the PAGE gel. (C) shows MS/MS analysis of the purified peptide.

FIG. 13 demonstrates production of N. meningitidis MC58 disaccharide, Gal(OAc)-DATDH, in E. coli. Silver-stained PAGE analysis of LOS form E. coli SCM7 harboring: Lane 1, plasmid p15 (expressing pglA-I) and empty vector (pACYC184); lane 2 plasmid p16 (contains C. jejuni pgl cluster with a transposon mutation in galE epimerase, abrogates production of UDP-GalNAc and therefore accumulates Undpp-diNAcBac) and empty vector (pMLBAD, p11); lane 3, plasmid p16 and plasmid p14 (expressing Shigella dysenteriae O1 rfpB, which encodes a galactosyltransferase); lane 4, plasmid p15 and plasmid p16. Cultures were induced with 0.2% Arabinose and grown overnight at 37° C. in a shaker flask, then cells were harvested and digested with proteinase K, followed by silver staining of amounts of sample (0.01 OD) as well as Western blotting of amounts of sample (0.01 OD). For both, samples were run on a 12% Bis-Tris NuPage SDS-gel (Invitrogen) using MES buffer for 50 min at 200V.

FIG. 14 demonstrates production of a glycoconjugate with N. meningitidis MC58 disaccharide, Gal(OAc)-DATDH, by combining C. jejuni N-glycan biosynthetic pathway and a Neisseria Galactosyltransferase. E. coli SCM6 was transformed with plasmids p15 (expressing pglA-I_{N. meningitidis}), p16 (pACYCga/Emut, expressing galE mutant of C. jejuni pgl), p9 (p114, expressing pglB_{C. jejuni}) and p6 (expressing engineered CTB, containing two N-glycosylation sites). (A) depicts a coommassie-stained SDS-PAGE of purified CTB using IMAC. (B) depicts an HPLC chromatogram of 2AB-labeled of LLO extracted from the cells after periplasmic extraction. (C) depicts MS analysis of tryptic digested glycosylated CTB. Terrific Broth (TB) was used for growing cells. Cultures were induced with 0.1% Arabinose and 1 mM IPTG, grown overnight at 37° C. in a shaker flask, then cells were harvested and the periplasmic proteins were extracted and applied to IMAC.

FIG. 15 demonstrates LLO analysis of engineered E. coli for production of recombinant glycoconjugate with N. meningitidis MC58 disaccharide, Gal(OAc)-DATDH. E. coli SCM6 was transformed with plasmids p17 (expressing synthetic operon pglFBCDAI_{N. meningitidis}), p9 (expressing pglB_{C. jejuni}) and p6 (expressing engineered CTB, containing two N-glycosylation sites). (A) depicts an HPLC chromatogram of 2AB-labeled of LLO extracted from the cells. (B) depicts MS/MS analysis of the peaks indicated by arrows in panel A. Terrific Broth (TB) supplemented with 10 mM MgCl₂ was used for growing cells. Cultures were induced with 0.1% Arabinose and 1 mM IPTG, grown overnight at 37° C. in a shaker flask, then cells were harvested and the periplasmic proteins were extracted and applied to IMAC.

FIG. 16 demonstrates production of a glycoconjugate with N. meningitidis MC58 disaccharide, Gal(OAc)-DATDH. E. coli SCM6 was transformed with plasmids p17 (expressing synthetic operon pglFBCDAI_{N. meningitidis}), p9 (expressing pglB_{C. jejuni}) p6 (expressing engineered CTB, containing two N-glycosylation sites). (A) depicts and coommassie-stained SDS-PAGE of the IMAC purified CTB. (B) depicts MS/MS analysis of the glycopeptides after trypsin digestion of glycosylated CTB. Upper left panel, ion fragmentations of glycosylated N₆₅GATFQVEVPGSDSNITHIDSQK₈₇ (peptide 1, SEQ ID NO:10) with Gal-DATDH; lower left panel, the same peptide with a miss-cleavage at position 87, N₆₅GATFQVEVPGSDSNITHIDSQKK₈₈ (peptide 2, SEQ ID NO:11) glycosylated with Gal(OAc)-DATDH; lower panels ion fragmentations of L₁₁₀CVWDNNK₁₁₇ (peptide 3, SEQ ID NO:12), with Gal-DATDH (left) and Gal(OAc) (right). Peptide 3 is carboxymethylated. Terrific Broth (TB) supplemented with 10 mM MgCl₂ was used for growing cells. Cultures were induced with 0.1% Arabinose and 1 mM IPTG, grown overnight at 37° C. in a shaker flask, then cells were harvested and the periplasmic proteins were extracted and applied to IMAC.

FIG. 17 demonstrates glycosylation of engineered EPA with N. meningitidis MC58 disaccharide, Gal(OAc)-DATDH. E. coli SCM6 was transformed with plasmids p17 (expressing synthetic operon pglFBCDAl_{N. meningitidis}), p9 (expressing pglB_{C. jejuni}) and p18 (expressing engineered EPA, containing four N-glycosylation sites). (A) depicts Western-blot analysis of the periplasmic extracts. Lane 1, unglycosylated EPA; lanes 2 and 3, glycosylated-EPA. (B) depicts coommassie-stained SDS-PAGE of the final purification step of glycosylated EPA, size-exclusion chromatography (SEC). Purified glycoproteins were digested with trypsin and subjected to LC-MS. (C) depicts MS/MS analysis of tryptic glycopeptides.

FIG. 18 demonstrates production of a glycoconjugate with N. meningitidis MC58 disaccharide, Gal-DATDH. E. coli SCM6 was transformed with plasmids p19 (expressing synthetic operon pgFBCDA_{N. meningitidis} lacking O-acetyltransfease (PglI)), p9 (expressing pglB_{C. jejuni}) and p6 (expressing engineered CTB, containing two N-glycosylation sites). (A) depicts an HPLC chromatogram of 2AB labeled LLO profile from cell pellets after periplasmic extraction, the arrow indicates the peak containing Gal-DATDH-2AB. (B) depicts coommassie-stained SDS-PAGE of the IMAC purified CTB. Lane 1, control negative, purified unglycosylated CTB; lanes 2-7, elution fractions from Ni-column; lane 8, control positive, purified CTB glycosylated with Gal(Oac)-DATDH. (C) depicts MS/MS analysis of the glycopeptides after trypsin digestion of glycosylated CTB. Ion fragmentation of glycosylated N₆₅GATFQVEVPGSDSNITHIDSQK₈₇ (SEQ ID NO:10) with Gal-DATDH is shown. Terrific Broth (TB) supplemented with 10 mM MgCl₂ was used for growing cells. Cultures were induced with 0.1% Arabinose and 1 mM IPTG, grown overnight at 37° C. in a shaker flask, then cells were harvested and the periplasmic proteins were extracted and applied to IMAC.

FIG. 19 demonstrates LLO analysis of engineered E. coli for production of recombinant conjugates with N. meningitidis disaccharide, Gal(OAc)-GATDH. E. coli SCM6 was transformed with plasmids p20 (expressing synthetic operon pglFB₂CDAI_{N. meningitidis}), p9 (expressing pglB_{C. jejuni}) and p6 (expressing engineered CTB, containing two N-glycosylation sites). (A) depicts an HPLC chromatogram of 2AB-labeled of LLO extracted from the cell, arrows show peaks containing the designated sugar structure. (B) depicts MS/MS analysis of the peaks indicated by arrows in panel A. Terrific Broth (TB) supplemented with 10 mM MgCl₂ was used for growing cells. Cultures were induced with 0.1% Arabinose and 1 mM IPTG, grown overnight at 37° C. in a shaker flask, then cells were harvested and the periplasmic proteins were extracted and applied to IMAC. LLO was extracted from cell pellets after the periplasmic extraction.

FIG. 20 demonstrates production of a glycoconjugate with N. meningitidis disaccharide, Gal(OAc)-GATDH. E. coli SCM6 was transformed with plasmids p20 (expressing synthetic operon pglFB₂CDAI_{N. meningitidis}), p9 (expressing pglB_{C. jejuni}) and p6 (expressing engineered CTB, containing two N-glycosylation sites). (A) depicts coommassie-stained SDS-PAGE of the IMAC purified CTB. Lane 1, unglycosylated purified CTB; lanes 2-7, elution fractions from Ni-column; lane 8, positive control (purified CTB di-glycosylated with Gal(OAc)-DATDH). (B) depicts MS/MS analysis of the glycopeptides after trypsin digestion of glycosylated CTB. A tryptic peptide, N₆₅GATFQVEVPGSDSNITHIDSQK₈₇ (SEQ ID NO:10), was identified to be glycosylated with Gal(OAc)-GATDH (upper panel) and non-acetylated form (lower panel). Terrific Broth (TB) supplemented with 10 mM MgCl₂ was used for growing cells. Cultures were induced with 0.1% Arabinose and 1 mM IPTG, grown overnight at 37° C. in a shaker flask, then cells were harvested and the periplasmic proteins were extracted and applied to IMAC.

FIG. 21 demonstrates production of a glycoconjugate with N. meningitidis disaccharide, Gal(OAc)-GATDH. E. coli SCM6 was transformed with plasmids p20 (expressing synthetic operon pglFB₂CDAI_{N. meningitidis}), p9 (expressing pgIB_{C. jejuni}) and p18 (expressing engineered EPA, containing four N-glycosylation sites). (A) depicts coommassie-stained SDS-PAGE of the purified EPA after size exclusion chromatography (SEC). Lane 1, unglycosylated purified EPA; lanes 2-7, elution fractions from a SEC column. (B) depicts MS/MS analysis of the glycopeptides after trypsin digestion of glycosylated EPA. A tryptic peptide, H₆₅DLDLIKDNNNSTPTVISHR₈₇ (SEQ ID NO:13), was identified to be glycosylated with Gal(OAc)-GATDH (upper panel) and non-acetylated form (lower panel). Terrific Broth (TB) supplemented with 10 mM MgCl₂ was used for growing cells. Cultures were induced with 0.1% Arabinose and 1 mM IPTG, grown overnight at 37° C. in a shaker flask, then cells were harvested and the periplasmic extract applied to anion exchange chromatography followed by SEC to obtain purified EPA.

FIG. 22 demonstrates specific glycan antibodies raised against different N. meningitidis O-glycosylated pilins recognize recombinant N-glycosylated proteins. Western-blot analysis of the glycosylated EPA (A) and CTB (B). Lane 1, unglycosylated protein; lane 2, glycosylated proteins with Gal(OAc)-DATDH; lane 3, with Gal-DATDH; lane 4, with Gal(OAc)-GATDH. 200 nanograms of purified protein was loaded to 12% NuPAGE Gel (Invitrogen) and run with MES Buffer for 70 min at 200V. Gels were trans blotted with iBlot and blocked by milk. Primary antibodies from rabbit, α-EPA (1:1000), α-CTB (1:1000), α-DATDH-Gal (1:20,000), α-GATDH (1:20,000) were used; HRP coupled anti-rabbit antibody (1:10,000) was used as the secondary antibody.

FIG. 23 demonstrates immunogenicity of CTB-DATDH-Gal(OAc) in rabbits. IgG titters were measured in rabbit sera after injection with recombinant glycoconjugates using ELISA. White New Zealand rabbits were injected three times, at 28 day time intervals, with low (1 μg) and high (10 μg) dosages of glycoconjugates with different adjuvants. 0.06% of Alhydrogel was used for rabbits injected with glycoconjugates adjuvenated with Alum. Freund's complete adjuvant (FCA) used for the first injection followed by Freund's incomplete adjuvant (FIC). Rabbit sera was obtained after 77 days. Control rabbits were injected with buffer containing adjuvants. Glycosylated EPA was used to coat the ELISA plate.

FIG. 24 demonstrates sera of rabbits injected with CTB-DATDH-Gal(OAc) recognizes Neisseria meningitidis glycosylated pilin. Western-blot analysis was performed on whole-cell extracts from different N. meningitidis strains: MØ1 240013, a clinical isolate with unknown O-glycan; NZ 98/254, containing glycosylated pilin with Gal(OAc)-GATDH; and H4476-SL and MC58, which both contain pilin glycosylated with Gal(OAc)-DATDH. Whole-cell extracts (0.09 OD) loaded to the gel blot were incubated with: (A), a serum containing polyclonal antibody against N. meningitidis pilin (1:2000); (B), a serum from a rabbit prior to injection with the conjugate (1:50); and (C) post immune serum (1:50).

FIG. 25 demonstrates glycosylation of the CTB by the disaccharide of C. jejuni. Western-Blot analysis of periplasmic extracts of SCM6 expressing CTB (“Plasmid 2”), PglJmut (“Plasmid 3,” lanes 1 and 2) or Pgl cluster (“Plasmid 1,” lane 3) and PglB overexpressed (“Plasmid 5,” lane 1 and 3) or the empty vector (eV, lane 2). Anti-CTB was used, 0.2 OD was loaded.

FIG. 26 demonstrates optimization of the glycosylation of the CTB. Western-Blot analysis against CTB of periplasmic extracts from (A) SCM6 expressing the CTB (“Plasmid 2”), PglB overexpressed (“Plasmid 5,” lanes 1 to 6, and 8) or not (eV, lane 7), the O-acetylated galactose of N. meningitidis (“Plasmid 12,” lane 1 to 6) and the pgl cluster mutated for GalE (“Plasmid 4,” lanes 1 to 6) or the WT cluster (“Plasmid 1,” lane 8) grown in LB with different antibiotics, induced for 4 h, 0.2 OD loaded; (B) SCM6 expressing the CTB, PglB overexpressed, the O-acetylated galactose of N. meningitidis, and the pgl cluster mutated for GalE, grown in LB, SOB, TB, TSB or BHI and induced for the night, 0.2 OD loaded. (C) Periplasmic extracts from the same strain grown in TB+/−10 mM MgCl₂ induced for the night and purified on a His SpinTrap column. Five μL of each fraction was loaded. L: Load, FT: Flow-Through, W: Wash, E: Elution fraction.

FIG. 27 demonstrates purification of the glycosylated CTB. SDS-PAGE analysis of the elution fraction from (A) SCM6 expressing the CTB (“Plasmid 2”), PglB overexpressed (“Plasmid 5”), the O-acetylated galactose of N. meningitidis (“Plasmid 12”) and the pgl cluster mutated for GalE (“Plasmid 4”); 5 μL of each fraction was loaded; (B) SCM6 expressing the CTB, PglB overexpressed, and the operon forming the entire disaccharide of N. meningitidis (“Plasmid 16,” pglF is in the wrong orientation); 10 μL of each fraction was loaded; (C) SCM6 expressing the CTB, PglB overexpressed, and the operon forming the entire disaccharide of N. meningitidis (“Plasmid 17,” pglF is in the correct orientation); 10 μL of each fraction was loaded. Gels were stained with Coomassie Simply Blue. For all the experiments, cells were grown in TB+10 mM MgCl₂ and induced overnight.

FIG. 28 depicts certain O-glycan structures that have been identified in Neisseria meningitidis (see BØrud et al., 2011, PNAS USA 108:9643-9648).

FIG. 29 depicts the general N-glycosylation pathway in C. jejuni. (A) shows genes that are involved in protein glycosylation, the Pgl cluster. (B) illustrates steps that are involved in biosynthesis of N-glycans and transferring N-glycans to protein acceptors.

FIG. 30 depicts the general O-glycosylation pathway in Neisseria. (A) shows the genetic organization of some of the genes that are involved in Neisseria pilin glycosylation. Unlike C. jejuni pgl, no single cluster of genes has been identified for Neisseria protein O-glycosylation. (B) illustrates steps that are involved in biosynthesis of 2,4-diacetimido-2,4,6-trideoxyhexopyranose (DATDH), assembly on undecaprenylpyrophosphate (Undpp), extension by a galactose residue as a function of PglA and O-acetylation with PglI, prior to flipping into the periplasm by PglF. After translocation into the periplasm PglL, the oligosaccharyltransferase, assembles the glycan en bloc onto a protein carrier such as pilin.

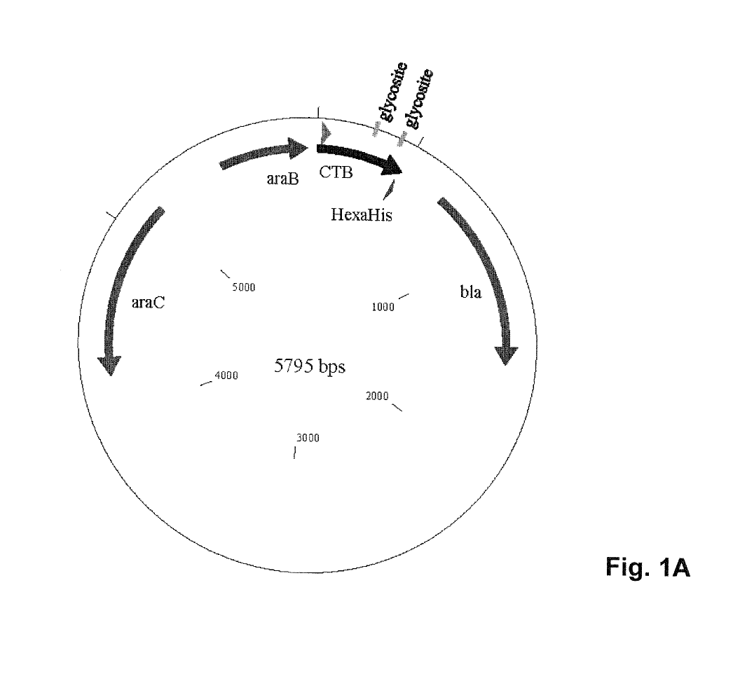

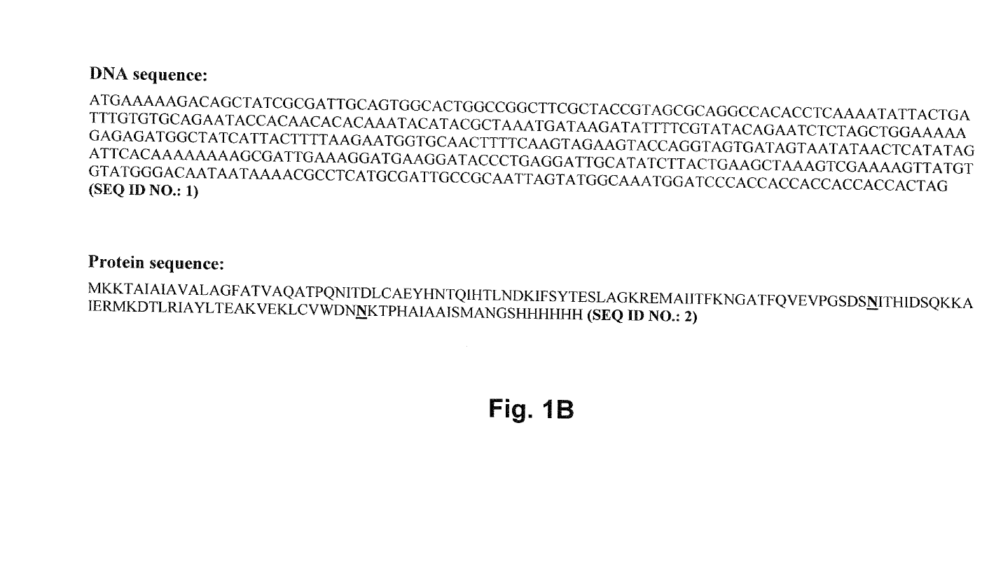

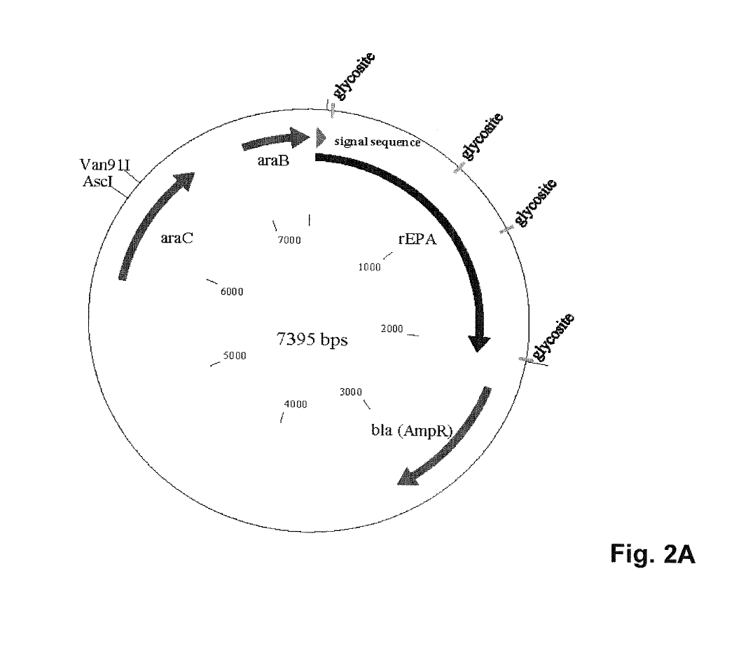

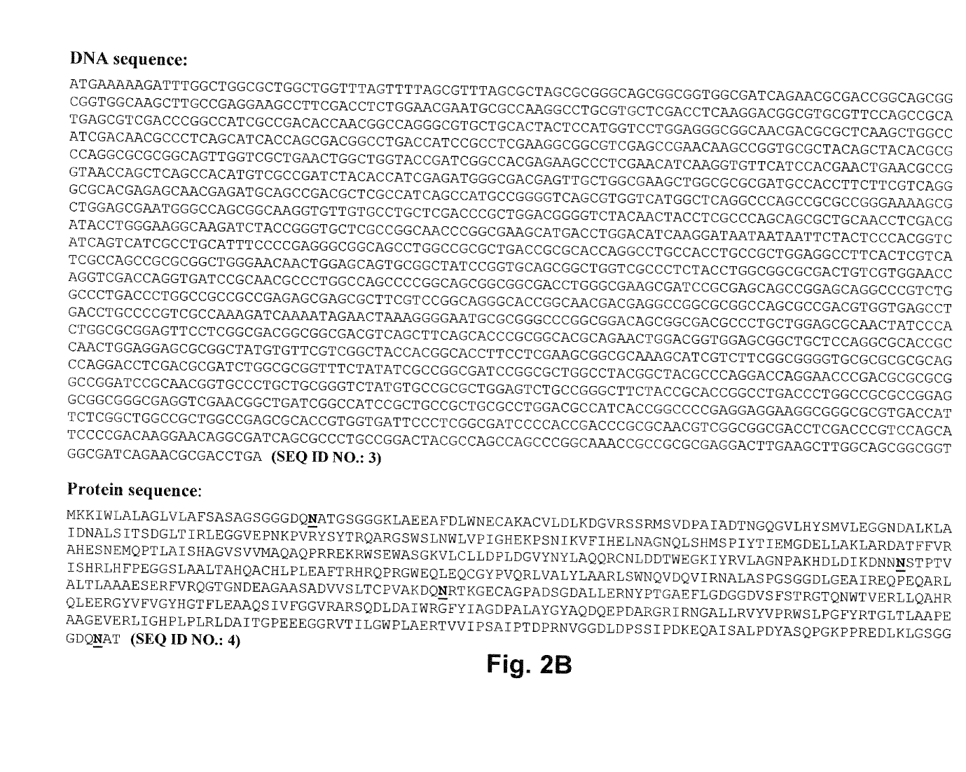

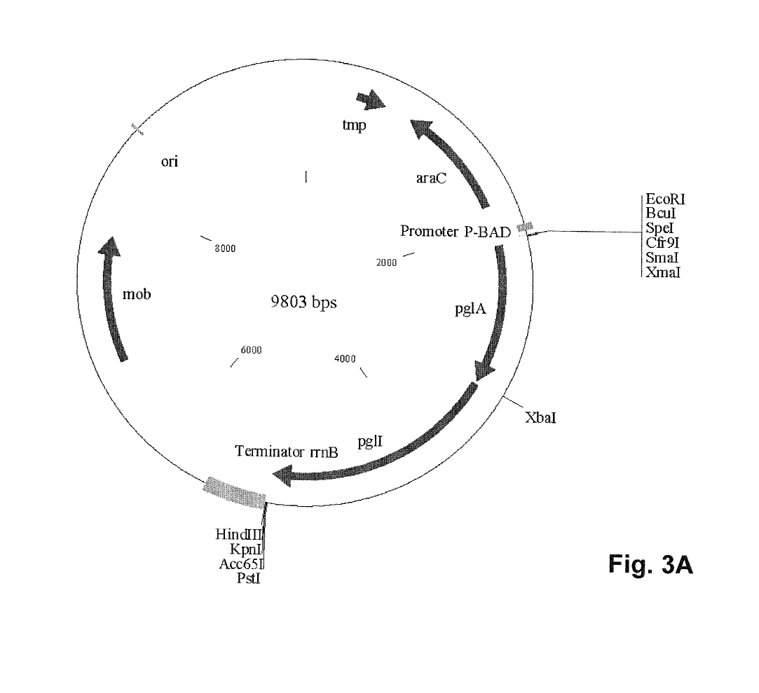

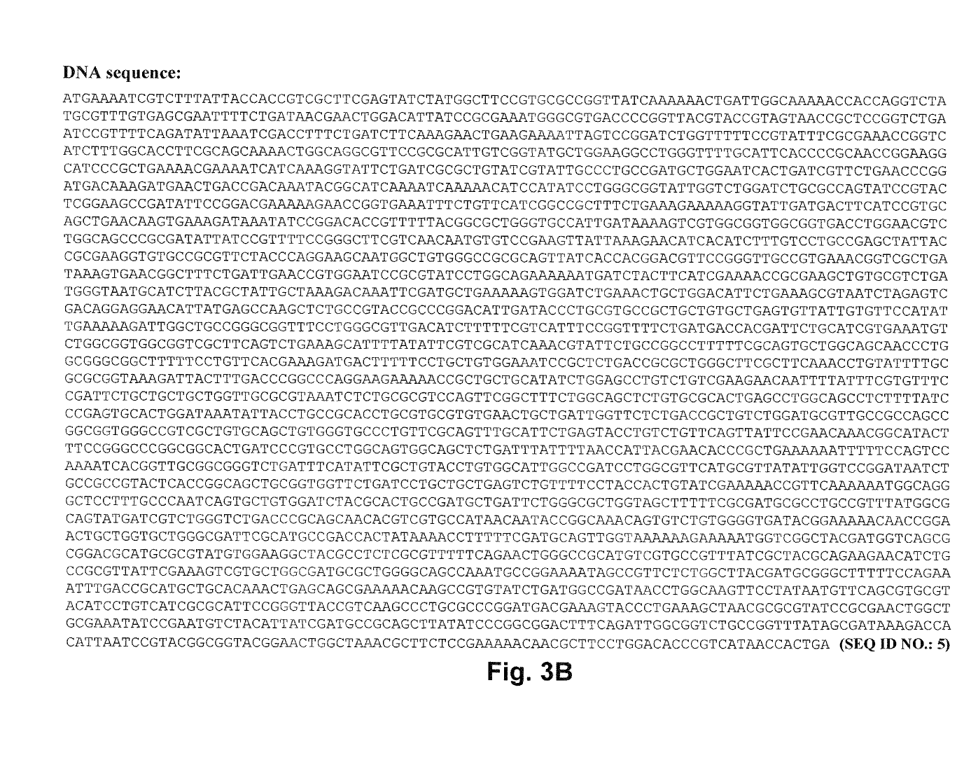

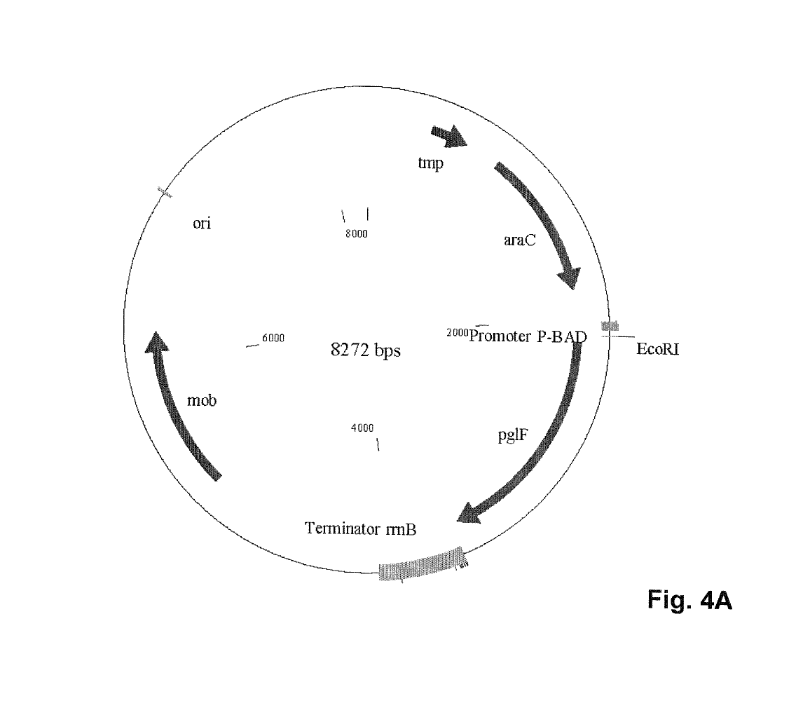

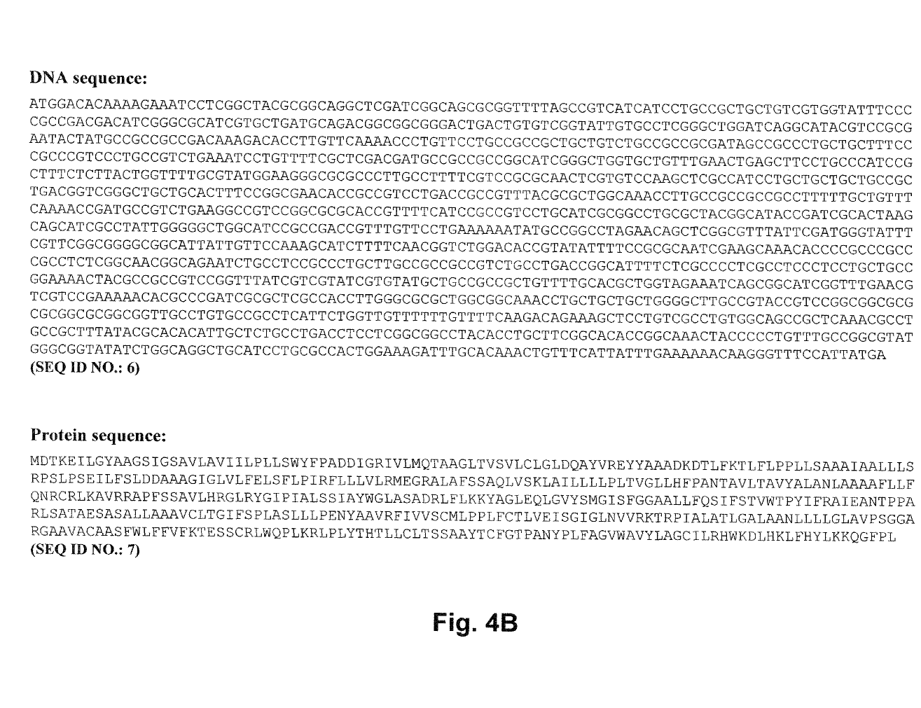

### 5. DETAILED DESCRIPTION

The present invention provides methods for production of short sugar moieties, e.g., monosaccharides, disaccharides, and trisaccharides, and assembly of such sugar moieties (e.g., monosaccharides, disaccharides, and trisaccharides) on a target (carrier) protein in prokaryotic systems In certain embodiments, carrier proteins are glycosylated, e.g. glycosylated at a glycosylation consensus sequence, for example, Asn-X-Ser/Thr, wherein X can be any amino acid except Pro; or an Asp/Glu-X-Asn-Z-Ser/Thr, wherein X and Z can be any amino acid except Pro, consensus sequence. In certain embodiments, one or more of such glycosylation consensus sequences can be introduced recombinantly into a protein of choice (a target/carrier protein).

Accordingly, the present invention also provides prokaryotic host cells that have been engineered (e.g., genetically manipulated using recombinant approaches) for use in accordance with the methods described herein. The present invention further provides bioconjugates, i.e., carrier proteins onto which are assembled short sugar moieties (e.g., monosaccharides, disaccharides, or trisaccharides), generated using the methods described herein. Further provided herein are compositions, e.g., immunogenic compositions, comprising the host cells and/or bioconjugates described herein. Such immunogenic compositions can be used, for example, as vaccines directed against the particular organisms from which the short sugar moieties (e.g., monosaccharides, disaccharides, or trisaccharides) used in accordance with the methods described herein are derived, e.g., vaccines against N. meningitidis and other bacterial species.

In certain embodiments, genes required for the biosynthesis of lipid linked oligosaccharides (LLO) are introduced into prokaryotic host cells that are used in accordance with the methods described herein, i.e., prokaryotic host cells capable of producing short sugar moieties (e.g., monosaccharides, disaccharides, or trisaccharides). In a specific embodiment, said oligosaccharides are Neisseria meningitidis oligosaccharides, i.e., the prokaryotic host cells described herein and used in accordance with the methods described herein comprise genes used in the biosynthesis of short sugar moieties (e.g., monosaccharides, disaccharides, or trisaccharides) of N. meningitidis. In certain embodiments, recombinant glycosyltransferases are introduced into the prokaryotic host cells used in the methods described herein. In specific embodiments, the glycosyltransferases synthesize the monosaccharide, disaccharide or trisaccharide on a lipid, such as undecaprenyl pyrophosphate. In certain specific embodiments, at least one glycosyltransferase is from a different organism than the prokaryotic host cell, that is, the glycosyltransferase is heterologous to (e.g., not normally associated with) the prokaryotic host cell. Such glycosyltransferases can be obtained from various sources, such as species of bacteria including Campylobacter and Neisseria. In certain embodiments, an oligosaccharyltransferase is introduced into the prokaryotic host. The oligosaccharyltransferases can be from any source, and may include heterologous oligosaccharyltransferases, i.e., oligosaccharyltransferases derived from a different organism than the prokaryotic host cell (e.g., oligosaccharyltransferases derived from a different bacterial species). In a specific embodiment, the oligosaccharyltransferase is from a species Campylobacter, e.g., C. jejuni.

In certain embodiments, the target protein onto which oligosaccharides are assembled in accordance with the methods described herein is Cholera toxin Subunit B (CTB) or exotoxin A of Pseudomonas aeruginusa (EPA).

The present invention further provides culture conditions for the glycosylation of the target proteins described herein by the methods described herein. For example, in certain embodiments, MgCl₂ is added to the culture medium, in particular 1 to 100 mM MgCl₂, 1 to 50 mM MgCl₂, 1 to 25 mM MgCl₂, 1 to 10 mM MgCl₂, 5 to 100 mM MgCl₂, 5 to 50 mM MgCl₂, 5 to 25 mM MgCl₂, 5 to 15 mM MgCl₂, at least 1 mM MgCl₂, at least 5 mM MgCl₂, at least 10 mM MgCl₂, at least 15 mM MgCl₂, at least 20 mM MgCl₂, or at least 25 mM MgCl₂ is added. In specific embodiments, at most 1 mM MgCl₂, at most 5 mM MgCl₂, at most 10 mM MgCl₂, at most 15 mM MgCl₂, at most 20 mM MgCl₂, or at most 25 mM MgCl₂ is added. In a specific embodiment, 10 mM MgCl₂ is added. In a specific embodiment, the MgCl₂ concentration in the culture medium is 10 mM.

In certain embodiments, Terrific Broth is used as a culture medium for protein glycoslyation.

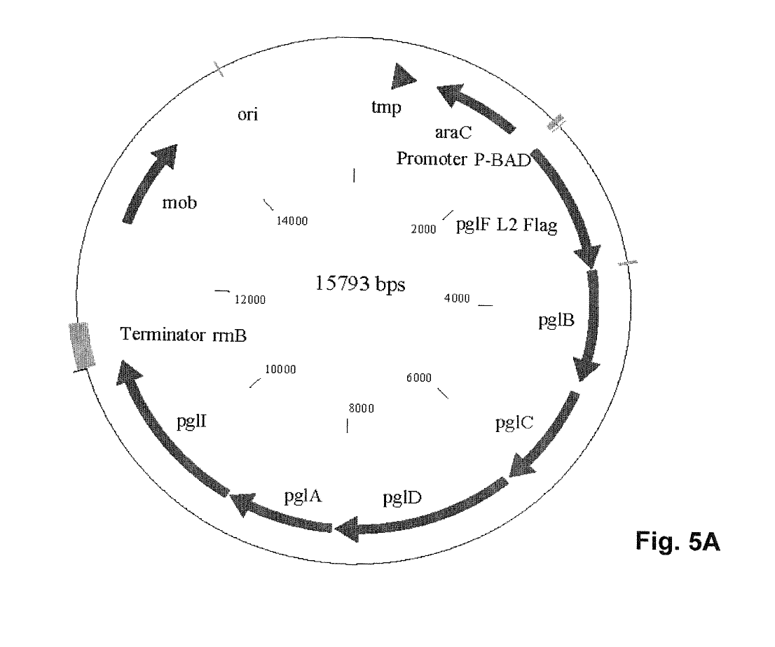

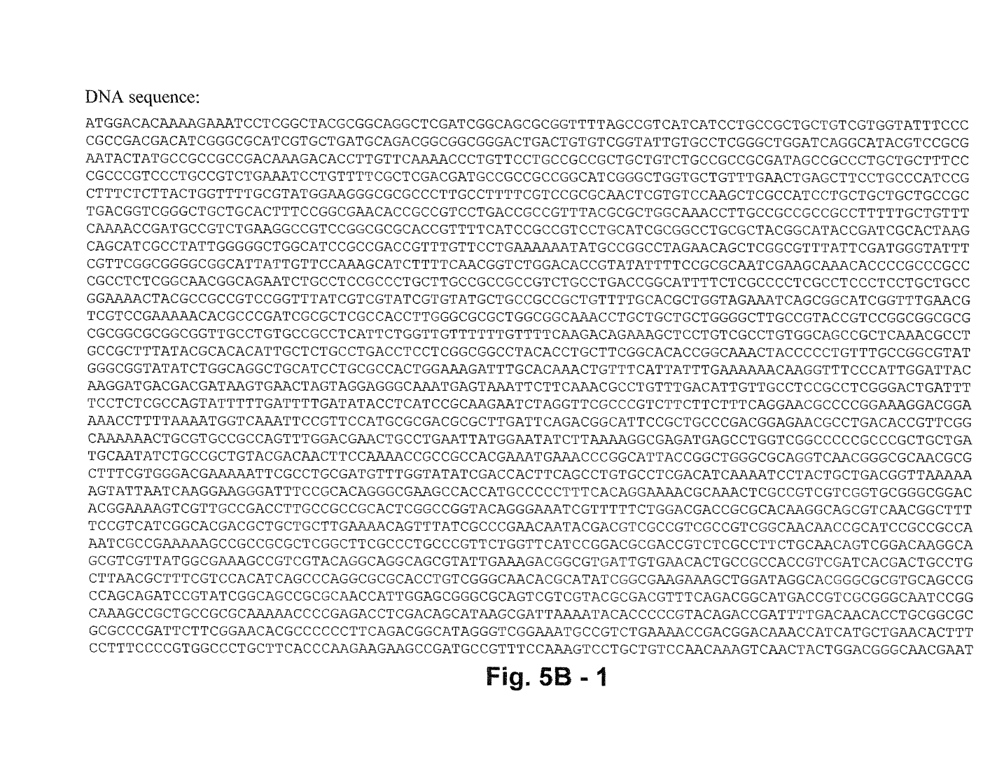

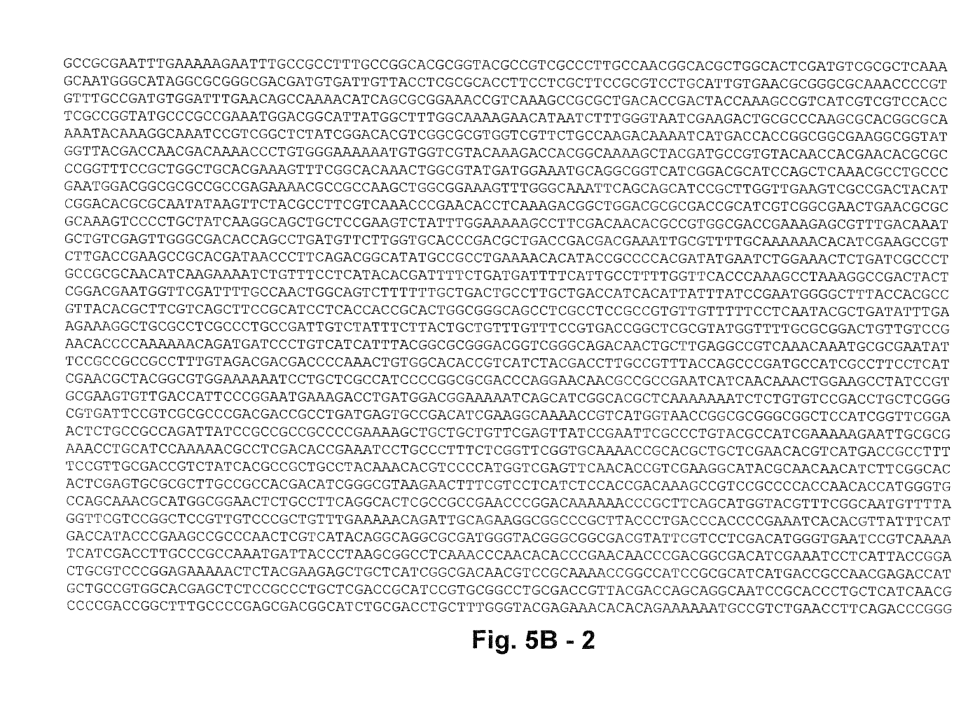

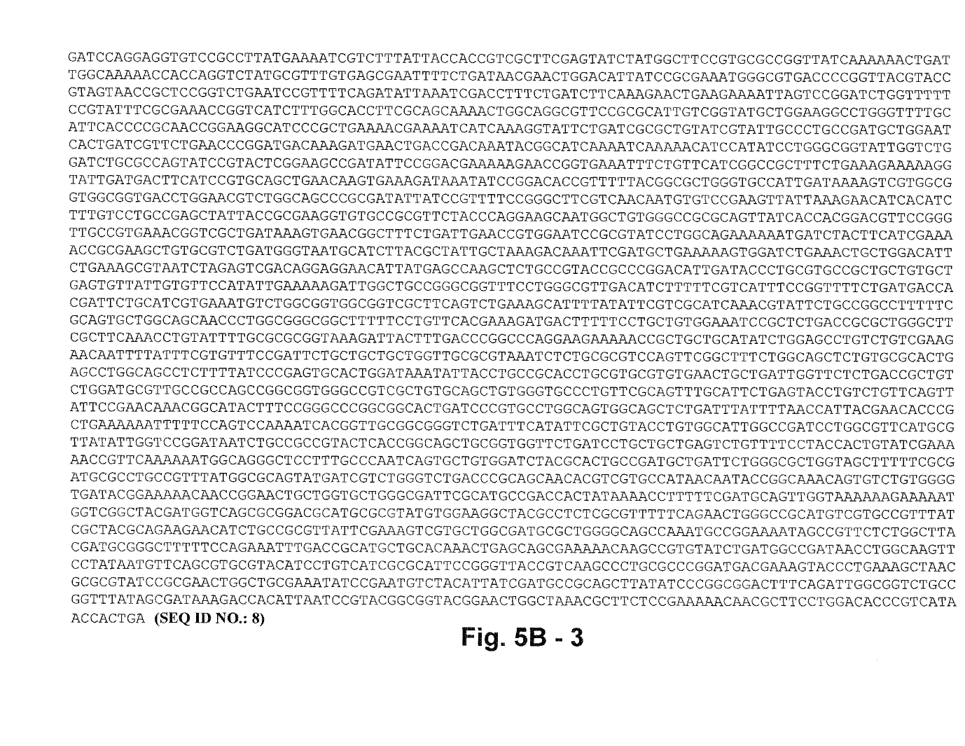

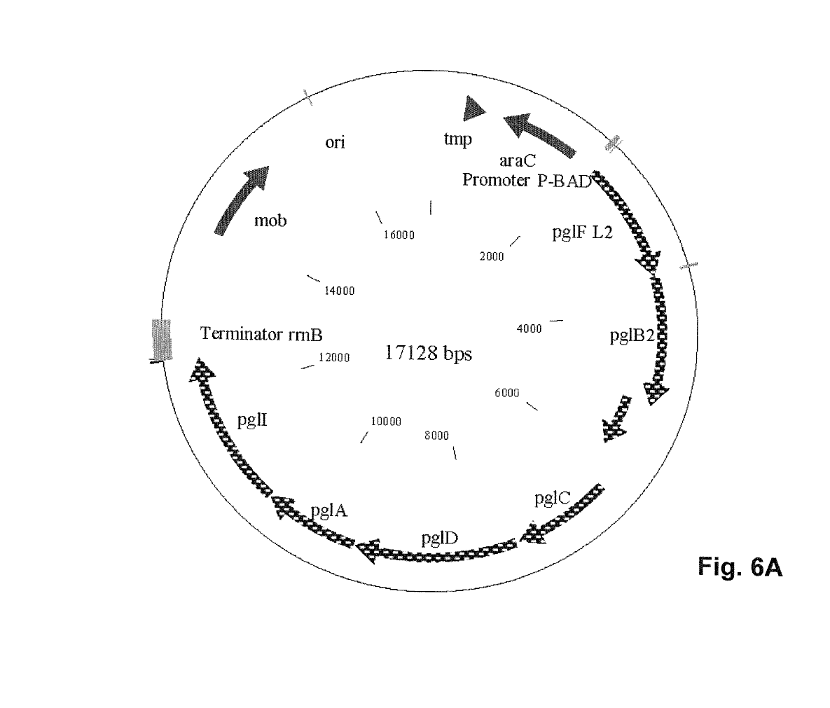

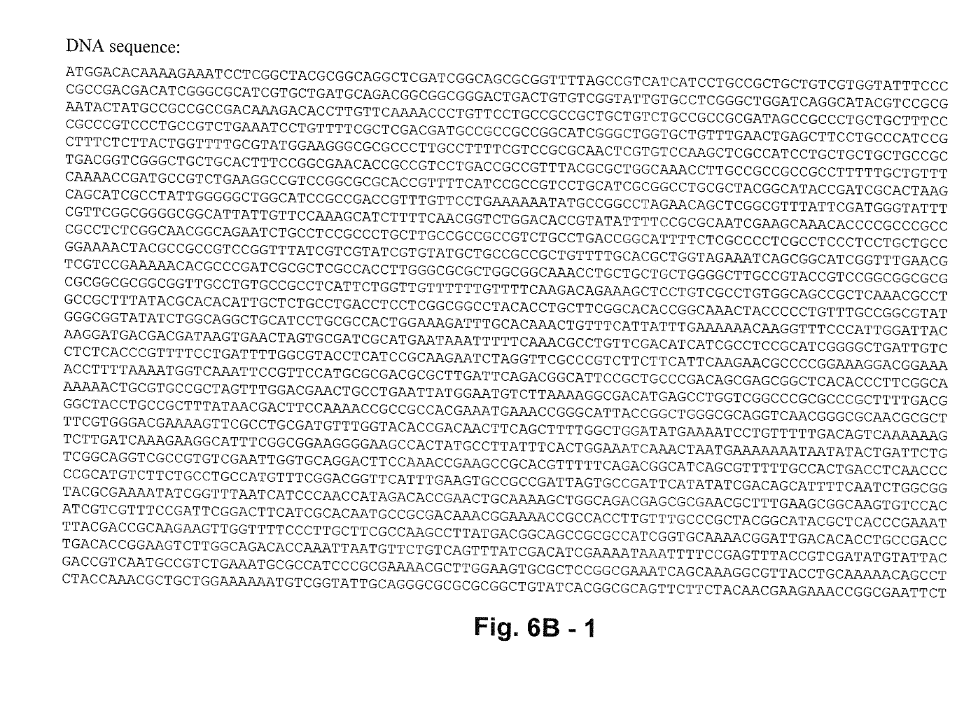

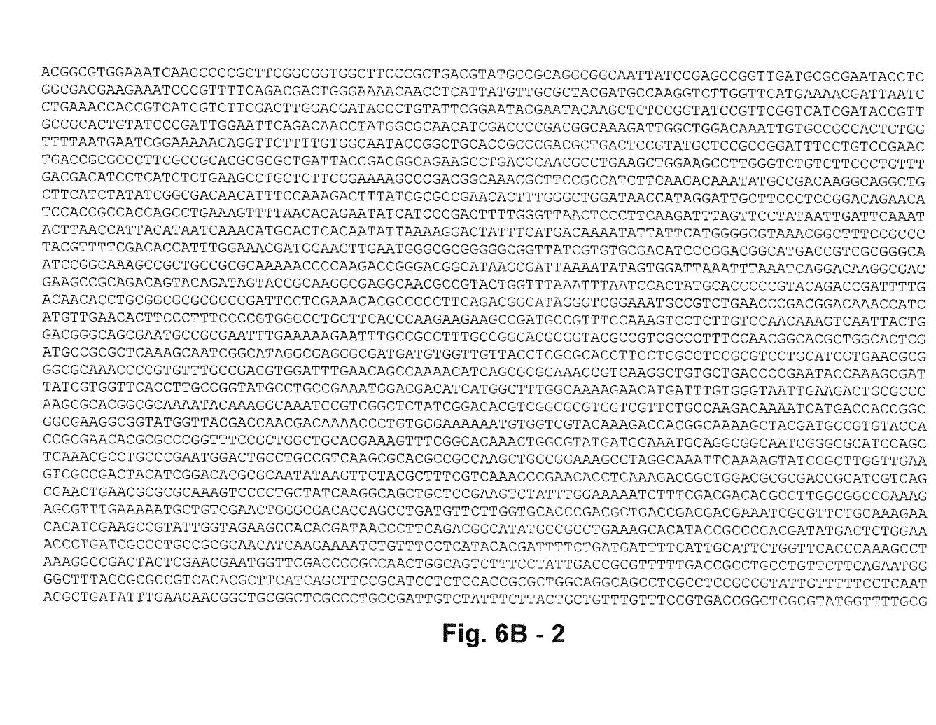

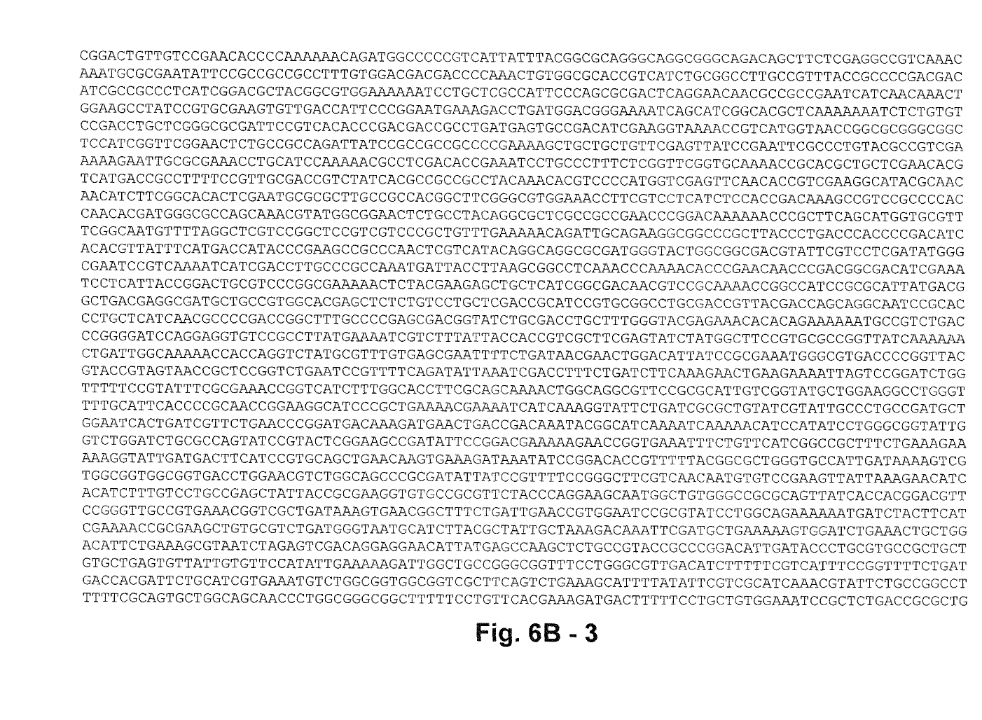

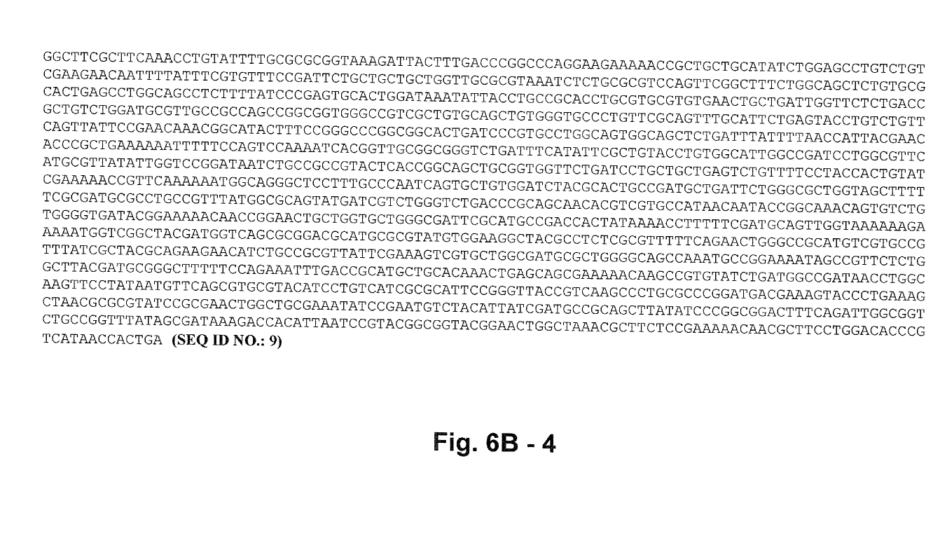

In certain embodiments, multiple open reading frames are introduced on a single plasmid to form a synthetic cluster for production of a specific lipid-linked oligosaccharide when said plasmid is introduced into a host prokaryotic cell (e.g., when the host cell is transformed with the plasmid). Without being bound by any particular theory of operation, reducing the number of plasmids by introducing a single synthetic cluster on one plasmid can be used to reduce the number of different antibiotics required to select for the presence of the plasmid. See FIGS. 5 and 6.

In specific embodiments, carrier proteins (e.g., CTB, EPA) glycosylated with a short sugar moiety (e.g., monosaccharide, disaccharide, or trisaccharide) of Neisseria meningitidis are used as an antigen, e.g. in an immunogenic composition. As demonstrated herein, glycan specific antibodies can be raised against proteins that were conjugated with oligosaccharide in accordance with the methods described herein. Thus, without being limited by and particular theory of operation, the bioconjugates described herein can be used to elicit immune responses in host organisms (e.g. human subjects) and thus represent vaccine candidates.

5.1 Host Cells

Any host cells can be used to produce bioconjugates in accordance with the methods described herein. In specific embodiments, the host cells used in accordance with the methods described herein are prokaryotic host cells. Exemplary prokaryotic host cells include, without limitation, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species. In a specific embodiment, the host cell used in accordance with the methods described herein is Escherichia coli (E. coli).

In certain embodiments, the host cells used in accordance with the methods described herein are engineered to comprise heterologous nucleic acids, e.g., heterologous nucleic acids that encode one or more carrier proteins (see, e.g., Section 5.2) and/or heterologous nucleic acids that encode one or more proteins, e.g., genes encoding one or more proteins (see, e.g., Section 5.1.1). In a specific embodiment, heterologous nucleic acids that encode proteins involved in glycosylation pathways (e.g., prokaryotic and/or eukaryotic glycosylation pathways) may be introduced into the host cells described herein. Such nucleic acids may encode proteins including, without limitation, oligosaccharyl transferases, glycosyltransferases, and/or flippases. Heterologous nucleic acids (e.g., nucleic acids that encode carrier proteins and/or nucleic acids that encode other proteins, e.g., proteins involved in glycosylation) can be introduced into the host cells described herein using any methods known to those of skill in the art, e.g., electroporation, chemical transformation by heat shock, natural transformation, phage transduction, and conjugation. In specific embodiments, heterologous nucleic acids are introduced into the host cells described herein on a plasmid, e.g., the heterologous nucleic acids are expressed in the host cells by a plasmid.

In certain embodiments, additional modifications may be introduced (e.g., using recombinant techniques) into the host cell that are useful for glycoprotein production. For example, host cell DNA can be removed that encodes a possibly competing or interfering pathway. In certain embodiments, the pathway to be removed is replaced by a desirable sequence, e.g., a sequence that is useful for glycoprotein production. Deletion of a gene that interferes with a desired activity is also an option, when the interfering activity is not replaced by a DNA insert. Exemplary genes that can be deleted include genes of the host cells involved in glycolipid biosynthesis, such as waaL (see, e.g., Feldman et al., 2005, PNAS USA 102:3016-3021), lipid A core biosynthesis cluster, galactose cluster, arabinose cluster, colonic acid cluster, capsular polysaccharide cluster, undecaprenol-p biosynthesis genes, und-P recycling genes, metabolic enzymes involved in nucleotide activated sugar biosynthesis, enterobacterial common antigen cluster, and prophage O antigen modification clusters like the gtrABS cluster.

5.1.1 Glycosylation Machinery

In certain embodiments, the glycosylation machinery of the host cell is engineered to produce a monosaccharide, a disaccharide, or a trisaccharide. In more specific embodiments, the glycosylation machinery of the host cell is engineered to produce a monosaccharide, a disaccharide, or a trisaccharide that would be part of a lipid-linked oligosaccharide of a prokaryotic cell such as Neisseria meningitidis. In even more specific embodiments, the glycosylation machinery of the host cell is engineered to produce a UndPP-linked monosaccharide, a disaccharide, or a trisaccharide.

Without being bound by theory, the UndPP-linked monosaccharide, a disaccharide, or a trisaccharide is then flipped from the cytosol of the host cell into the periplasmic space of the host cell. Further, without being bound by theory, the monosaccharide, a disaccharide, or a trisaccharide is then transferred from UndPP onto the carrier protein on an Asn of a glycosylation site of the carrier protein.

In certain embodiments, a glycosyltransferase of the host cell's glycosylation machinery is functionally inactivated by recombinant means so that the synthesis of a lipid-linked oligosaccharide is interrupted resulting in a monosaccharide, a disaccharide, or a trisaccharide. In addition, other components of the glycosylation machinery can be modified or inactivated to modify the chemical structure of the resulting monosaccharide, a disaccharide, or a trisaccharide.

In certain embodiments, a heterologous nucleic acid encoding a glycosyltransferase is introduced into the host cell so that a monosaccharide, a disaccharide, or a trisaccharide is generated on UndPP. In certain, more specific embodiments, a heterologous glycosylation operon is introduced into the host cell wherein the heterologous glycosylation operon is mutated such that upon expression of its enzymes a UndPP-linked monosaccharide, a disaccharide, or a trisaccharide is generated in the host cell. In certain specific embodiments, the heterologous glycosyltransferase is PglA of Neisseria meningitidis or RfpB of Shigella dysenteriae.

In certain specific embodiments, the host cell has been engineered to produce Und-PP-DATDH. In even more specific embodiments, PglB, PglC, and/or PglD of Neisseria meningitidis are introduced into the host cell. In certain specific embodiments, the host cell has been engineered to produce Und-PP-GATDH. In even more specific embodiments, PglB2, PglC, and/or PglD of Neisseria meningitidis are introduced into the host cell. In even more specific embodiments, PglB, PglC, PglD, PglF, PglA, and/or PglI of Neisseria meningitidis are introduced into the host cell. In even more specific embodiments, PglB2, PglC, PglD, PglF, PglA, and/or PglI of Neisseria meningitidis are introduced into the host cell.

5.2 Carrier Proteins

Any carrier proteins suitable for use in the methods described herein can be used in accordance with the methods described herein. Exemplary carrier proteins include, without limitation, Exotoxin A of P. aeruginosa, CRM197, Diphtheria toxoid, tetanus toxoid, detoxified hemolysin A of S. aureus, clumping factor A, clumping factor B, E. coli FimH, E. coli FimHC, E. coli heat labile enterotoxin, detoxified variants of E. coli heat labile enterotoxin, Cholera toxin B subunit, cholera toxin, detoxified variants of cholera toxin, E. coli sat protein, the passenger domain of E. coli sat protein, C. jejuni AcrA, and a C. jejuni natural glycoprotein.

In a specific embodiment, the carrier protein to generate a bioconjugate described herein is an antigen of Neisseria, e.g., an antigen of Neisseria meningitidis such as an antigen of N. meningitidis group B. Exemplary antigens of N. meningitidis include, without limitation, pilin, NMB0088, nitrite reductase (AniA), heparin-binding antigen (NHBA), factor H binding protein (fHBP), adhesion, NadA, Ag473, or surface protein A (NapA).

In certain embodiments, the carrier proteins used in accordance with the methods described herein are modified, e.g., modified in such a way that the protein is less toxic and or more susceptible to glycosylation, etc. In a specific embodiment, the carrier proteins used in the methods described herein are modified such that the number of glycosylation sites in such proteins is maximized in a manner that allows for lower concentrations of the protein to be administered, e.g., in an immunogenic composition, in its bioconjugate form. Accordingly in certain embodiments, the carrier proteins described herein are modified to include 1, 2, 3, 4, 5, 6, 7, 8, 9, 10, or more glycosylation sites than would normally be associated with the carrier protein (e.g., relative to the number of glycosylation sites associated with the carrier protein in its native/natural, e.g., “wild-type” state). In specific embodiments, introduction of glycosylation sites is accomplished by insertion of glycosylation consensus sequences anywhere in the primary structure of the protein. Introduction of such glycosylation sites can be accomplished by, e.g., adding new amino acids to the primary structure of the protein (i.e., the glycosylation sites are added, in full or in part), or by mutating existing amino acids in the protein in order to generate the glycosylation sites (i.e., amino acids are not added to the protein, but selected amino acids of the protein are mutated so as to form glycosylation sites). Those of skill in the art will recognize that the amino acid sequence of a protein can be readily modified using approaches known in the art, e.g., recombinant approaches, that include modification of the nucleic acid sequence encoding the protein. In specific embodiments, glycosylation consensus sequences are introduced into surface structures of the protein, at the N or C termini of the protein, and/or in loops that are stabilized by disulfide bridges at the base of the protein. In certain embodiments, the classical 5 amino acid consensus may be extended by Lysine residues for more efficient glycosylation, and thus the inserted consensus sequence may encode 5, 6, or 7 amino acids that should be inserted or that replace acceptor protein amino acids.

In certain embodiments, the carrier proteins used in accordance with the methods described herein comprise a “tag,” i.e., a sequence of amino acids that allow for the isolation and/or identification of the carrier protein. For example, adding a tag to a carrier protein described herein can be useful in the purification of that protein. Exemplary tags that can be used herein include, without limitation, histidine (HIS) tags (e.g., hexa histidine-tag, or 6×His-Tag), FLAG-TAG, and HA tags. In certain embodiments, the tags used herein are removable, e.g., removal by chemical agents or by enzymatic means, once they are no longer needed, e.g., after the protein has been purified.

5.3 Sugar Moiety

The instant invention relates, in part, to Applicants discovery that host cells, e.g., prokaryotic host cells, can be engineered using recombinant approaches to produce bioconjugates comprising carrier proteins onto which are assembled short sugar moieties (i.e., at glycosylation sites on the carrier proteins). Accordingly, encompassed herein are bioconjugates comprising a carrier protein and any short sugar moiety known to those of skill in the art. In a specific embodiment, a monosaccharide is assembled on a carrier protein described herein. In another specific embodiment, a disaccharide is assembled on a carrier protein described herein. In another specific embodiment, a trisaccharides is assembled on a carrier protein described herein.

Those of skill in the art will recognize that based on the discovery of the instant invention, monosaccharides can be transferred to carrier proteins. Thus, those of skill in the art will recognize that the particular monosaccharides selected for use in accordance with the methods described herein are not limited. In certain embodiments, the monosaccharide is DATDH or GATDH. In another specific embodiment, provided herein is a bioconjugate comprising a carrier protein and a monosaccharide, wherein the monosaccharide is from N. meningitidis.

Further, those of skill in the art will recognize that based on the discovery of the instant invention, disaccharides can be transferred to carrier proteins. Thus, those of skill in the art will recognize that the particular disaccharides selected for use in accordance with the methods described herein are not limited. In certain embodiments, the disaccharide is Gal-DATDH, Gal(OAc)-DATDH, Gal-GATDH, Gal-GATDH, Gal(OAc)-GATDH, Gal-GlcNAc, Gal(OAc)-GlcNAc, Glc-DATDH, or Glc-GATDH. In another specific embodiment, provided herein is a bioconjugate comprising a carrier protein and a disaccharide, wherein the disaccharide is from N. meningitidis.

Further still, those of skill in the art will recognize that based on the discovery of the instant invention, trisaccharides can be transferred to carrier proteins. Thus, those of skill in the art will recognize that the particular trisaccharides selected for use in accordance with the methods described herein are not limited. In certain embodiments, the trisaccharide is Gal(OAc)-Gal-DATDH, Gal-Gal-DATDH, Gal(OAc)-Gal-GATDH, or Gal-Gal-GATDH. In another specific embodiment, provided herein is a bioconjugate comprising a carrier protein and a trisaccharide, wherein the trisaccharide is from N. meningitidis.

In certain embodiments, the monosaccharide, disaccharide, or trisaccharide of the bioconjugates provided herein is covalently bound to the Asn within a glycosylation site of the carrier protein, wherein the glycosylation site comprises the amino acid sequence Asp/Glu-X-Asn-Z-Ser/Thr wherein X and Z may be any amino acid except Pro. In certain embodiments, the carrier proteins of the bioconjugates provided herein do not naturally (e.g., in their normal/native, or “wild-type” state) comprise a glycosylation site. In certain embodiments, the carrier proteins of the bioconjugates provided herein are engineered to comprise one or more glycosylation sites, e.g., the carrier proteins are engineered to comprise one or more glycosylation sites comprising the amino acid sequence Asp/Glu-X-Asn-Z-Ser/Thr wherein X and Z may be any amino acid except Pro. For example, the carrier proteins used in accordance with the methods described herein may comprise 1, 2, 3, 4, 5, 6, 7, 8, 9, 10 or more glycosylation sites, each having the amino acid sequence Asp/Glu-X-Asn-Z-Ser/Thr, wherein X and Z may be any amino acid except Pro; and wherein some (e.g., 1, 2, 3, 4, 5, 6, 7, 8, or 9) or all of the glycosylation sites have been recombinantly introduced into the carrier protein.

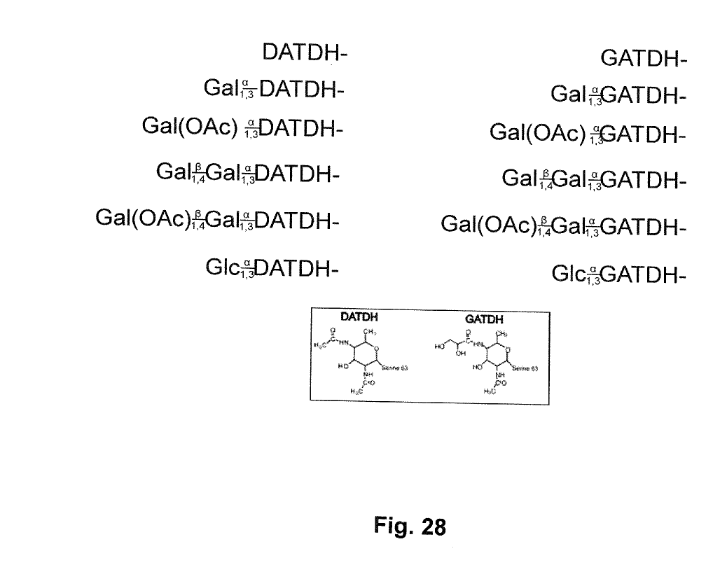

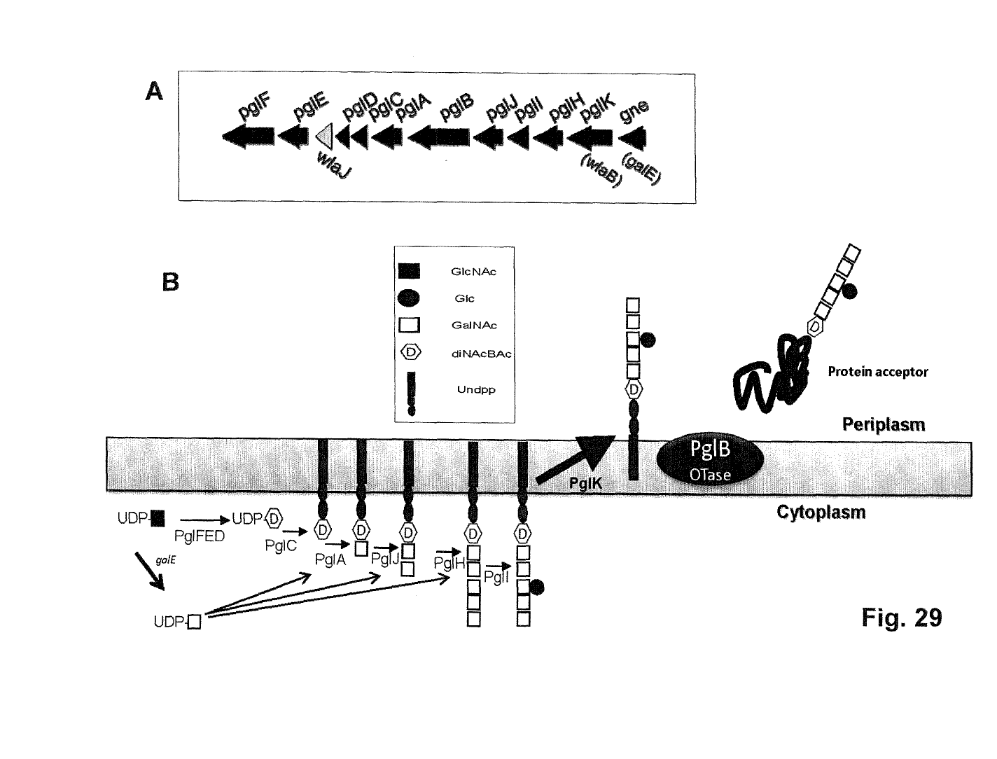

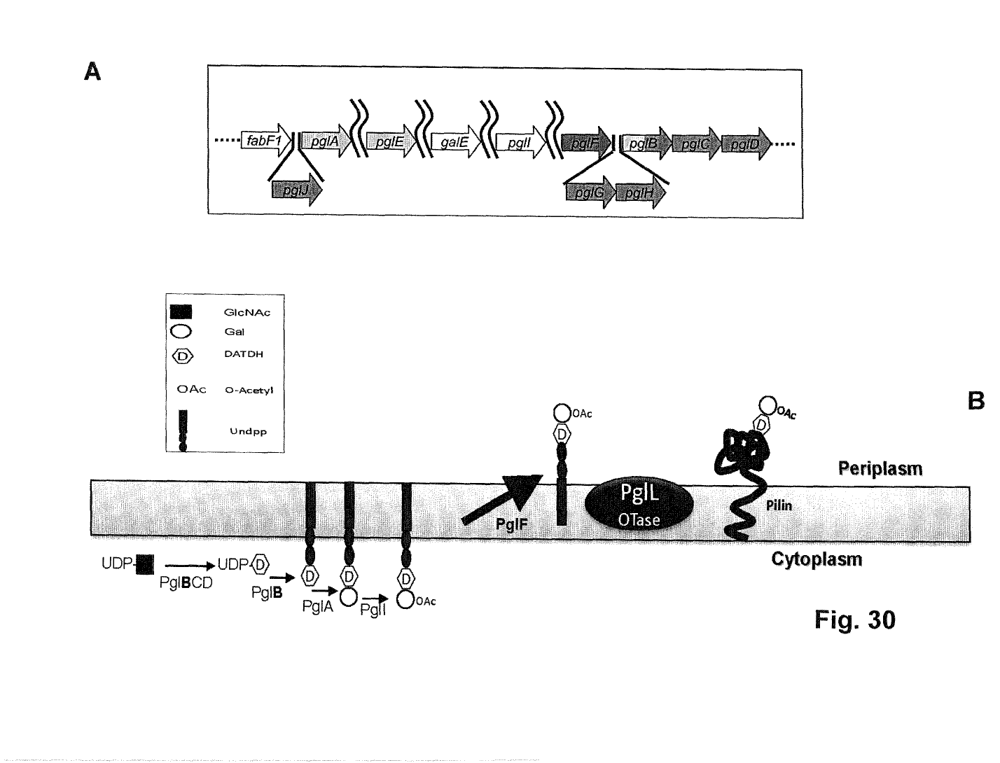

In a specific embodiment, the short sugar moiety attached to a carrier protein to form a bioconjugate described herein is a sugar moiety illustrated in FIG. 28, e.g., the sugar moiety has the same structure and linkages as one of the sugar moieties illustrated in FIG. 28.

In specific embodiments, the sugar moieties attached to a carrier protein to form a bioconjugate described herein comprise the same linkage. In a specific embodiment, the sugar moieties attached to a carrier protein to form a bioconjugate described herein comprise α1,3 linkages. In another specific embodiment, the sugar moieties attached to a carrier protein to form a bioconjugate described herein comprise α1,4 linkages. In another specific embodiment, the sugar moieties attached to a carrier protein to form a bioconjugate described herein comprise α1,3 and α1,4 linkages. See FIG. 28.

In specific embodiments, the sugar moieties attached to a carrier protein to form a bioconjugate described herein have the same chirality. In specific embodiments, the sugars comprise D isomers.

In specific embodiments, the reducing end of the short sugar moieties (e.g., monosaccharides, disaccharides, and trisaccharides) described herein has a specific component. In a specific embodiment, the reducing end of the short sugar moieties (e.g., monosaccharides, disaccharides, and trisaccharides) comprises a galactose.

5.4 Bioconjugates

Provided herein are bioconjugates produced by the host cells described herein, wherein said bioconjugates comprise a carrier protein and a monosaccharide, disaccharide, and/or trisaccharide. As referred to herein, bioconjugates comprise a carrier protein and short sugar moiety (e.g., a monosaccharide, disaccharide, and/or trisaccharide), wherein said short sugar moiety (e.g., a monosaccharide, disaccharide, and/or trisaccharide) is covalently linked to an asparagine (ASN) residue of the carrier protein (e.g., linked at a glycosylation site of the carrier protein).

In a specific embodiment, provided herein is a bioconjugate comprising a carrier protein and a monosaccharide, e.g., DATDH or GATDH. In another specific embodiment, provided herein is a bioconjugate comprising a carrier protein and a monosaccharide, wherein the monosaccharide is from N. meningitidis. In another specific embodiment, the carrier protein is CTB, EPA, or an antigen of N. meningitidis.

In another specific embodiment, provided herein is a bioconjugate comprising a carrier protein and a disaccharide, e.g., Gal-DATDH, Gal(OAc)-DATDH, Gal-GATDH, Gal-GATDH, Gal(OAc)-GATDH, Gal-GlcNAc, Gal(OAc)-GlcNAc, Glc-DATDH, or Glc-GATDH. In another specific embodiment, provided herein is a bioconjugate comprising a carrier protein and a disaccharide, wherein the disaccharide is from N. meningitidis. In another specific embodiment, the carrier protein is CTB, EPA, or an antigen of N. meningitidis.

In another specific embodiment, provided herein is a bioconjugate comprising a carrier protein and a trisaccharide, e.g., Gal(OAc)-Gal-DATDH, Gal-Gal-DATDH, Gal(OAc)-Gal-GATDH, or Gal-Gal-GATDH. In another specific embodiment, provided herein is a bioconjugate comprising a carrier protein and a trisaccharide, wherein the trisaccharide is from N. meningitidis. In another specific embodiment, the carrier protein is CTB, EPA, or an antigen of N. meningitidis.

In certain embodiments, the bioconjugates provided herein are isolated, i.e., the bioconjugates are produced by a host cell described herein using methods of production of bioconjugates known in the art and/or described herein, and the produced bioconjugate is isolated and/or purified. In certain embodiments, the bioconjugates provided herein are at least 75%, 80%, 85%, 90%, 95%, 98%, or 99% pure, e.g., free from other contaminants, etc.

In certain embodiments, the bioconjugates provided herein are homogeneous with respect to the short sugar moieties attached to the glycosylation sites of the bioconjugates, e.g., the bioconjugates express all of the same monosaccharide, all of the same disaccharide, all of the same trisaccharide, etc., at the glycosylation sites of the bioconjugate.

In certain embodiments, the bioconjugates provided herein are not homogeneous with respect to the short sugar moieties attached to the glycosylation sites of the bioconjugates, e.g., the bioconjugates express different monosaccharides, different disaccharides, and/or different trisaccharides, or combinations thereof (e.g., some monosaccharides and some disaccharides), at the glycosylation sites of the bioconjugate.

In certain embodiments, the bioconjugates provided herein possess greater than one glycosylation site, wherein each glycosylation site of the bioconjugate is glycosylated (i.e., 100% of the glycosylation sites of the bioconjugate are glycosylated). In certain embodiments, the bioconjugates provided herein possess greater than one glycosylation site, wherein not all of the glycosylation sites of the bioconjugate are glycosylated, e.g., about or at least 10%, 20%, 25%. 30%, 35%, 40%, 45%, 50%, 55%, 60%, 65%, 70%, 75%, 80%, 85%, 90%, or 95% of the glycosylation sites of the bioconjugate are glycosylated, but not all of the glycosylation sites of the bioconjugate are glycosylated. In certain embodiments, all of the glycosylation sites of the bioconjugate that are glycosylated comprise (i.e., are glycosylated with) the same monosaccharide, the same disaccharide, or the same trisaccharide.

In certain embodiments, provided herein are populations of bioconjugates. In one embodiment, provided herein is a population of bioconjugates, wherein at least 50%, 55%, 60%, 65%, 70%, 75%, 80%, 85%, 90%, or 95%, or wherein 100%, of a first glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the first glycosylation site of each bioconjugate is glycosylated with the same monosaccharide, disaccharide, or trisaccharide as the other bioconjugates in the population (i.e., all bioconjugates have the same sugar moiety at the first glycosylation site of the carrier protein). In certain embodiments, at least 50%, 55%, 60%, 65%, 70%, 75%, 80%, 85%, 90%, or 95%, or 100%, of a second glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the second glycosylation site of each bioconjugate is glycosylated with the same monosaccharide, disaccharide, or trisaccharides as the other bioconjugates in the population (i.e., all bioconjugates have the same sugar moiety at the second glycosylation site of the carrier protein). In certain embodiments, at least 50%, 55%, 60%, 65%, 70%, 75%, 80%, 85%, 90%, or 95%, or 100%, of a third glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the third glycosylation site of each bioconjugate is glycosylated with the same monosaccharide, disaccharide, or trisaccharides as the other bioconjugates in the population (i.e., all bioconjugates have the same sugar moiety at the third glycosylation site of the carrier protein). In certain embodiments, at least 50%, 55%, 60%, 65%, 70%, 75%, 80%, 85%, 90%, or 95%, or 100%, of a fourth glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the fourth glycosylation site of each bioconjugate is glycosylated with the same monosaccharide, disaccharide, or trisaccharides as the other bioconjugates in the population (i.e., all bioconjugates have the same sugar moiety at the fourth glycosylation site of the carrier protein). In certain embodiments, at least 50%, 55%, 60%, 65%, 70%, 75%, 80%, 85%, 90%, or 95%, or 100%, of a fifth glycosylation site in the carrier protein of the bioconjugates in the population is glycosylated. In a specific embodiment, at least 50%, 55%, 60%, 65%, 70%, 75%, 80%, 85%, 90%, or 95%, or 100%, of the fifth glycosylation site of each bioconjugate is glycosylated with the same monosaccharide, disaccharide, or trisaccharides as the other bioconjugates in the population (i.e., all bioconjugates have the same sugar moiety at the fifth glycosylation site of the carrier protein).

5.5 Compositions

5.5.1 Compositions Comprising Host Cells

In one embodiment, provided herein are compositions comprising the host cells described herein. Such compositions can be used in methods for generating the bioconjugates described herein, e.g., the compositions can be cultured under conditions suitable for the production of proteins. Subsequently, the bioconjugates can be isolated from said compositions.

The compositions comprising the host cells provided herein can comprise additional components suitable for maintenance and survival of the host cells described herein, and can additionally comprise additional components required or beneficial to the production of proteins by the host cells, e.g., inducers for inducible promoters, such as arabinose, IPTG.

5.5.2 Compositions Comprising Bioconjugates

In another embodiment, provided herein are compositions comprising the bioconjugates described herein. Such compositions can be used in methods of treatment and prevention of disease.

In a specific embodiment, provided herein are immunogenic compositions comprising one or more of the bioconjugates described herein. The immunogenic compositions provided herein can be used for eliciting an immune response in a host to whom the composition is administered. Thus, the immunogenic compositions described herein can be used as vaccines and can accordingly be formulated as pharmaceutical compositions.

The compositions comprising the bioconjugates described herein may comprise any additional components suitable for use in pharmaceutical administration. In specific embodiments, the immunogenic compositions described herein are monovalent formulations. In other embodiments, the immunogenic compositions described herein are multivalent formulations. For example, a multivalent formulation comprises more than one bioconjugate described herein.

In certain embodiments, the compositions described herein additionally comprise a preservative, e.g., the mercury derivative thimerosal. In a specific embodiment, the pharmaceutical compositions described herein comprises 0.001% to 0.01% thimerosal. In other embodiments, the pharmaceutical compositions described herein do not comprise a preservative.

In certain embodiments, the immunogenic compositions described herein comprise, or are administered in combination with, an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to a bioconjugate, but when the compound is administered alone does not generate an immune response to the bioconjugate. In some embodiments, the adjuvant generates an immune response to the poly bioconjugate peptide and does not produce an allergy or other adverse reaction. Adjuvants can enhance an immune response by several mechanisms including, e.g., lymphocyte recruitment, stimulation of B and/or T cells, and stimulation of macrophages.

5.6 Uses

In one embodiment, provided herein are methods for inducing an immune response in a subject comprising administering to the subject a bioconjugate described herein or a composition thereof. In a specific embodiment, a method for inducing an immune response to a bioconjugate described herein comprises administering to a subject in need thereof an effective amount of a bioconjugate described herein or a composition thereof.

In a specific embodiment, the subjects to whom a bioconjugate or composition thereof is administered have, or are susceptible to, an infection, e.g., a bacterial infection. In a specific embodiment, the subjects to whom a bioconjugate or composition thereof is administered are susceptible to infection with Neisseria meningitidis, e.g., Neisseria meningitidis group B, i.e., the bioconjugate is administered as a vaccine against Neisseria meningitidis, e.g., Neisseria meningitidis group B.

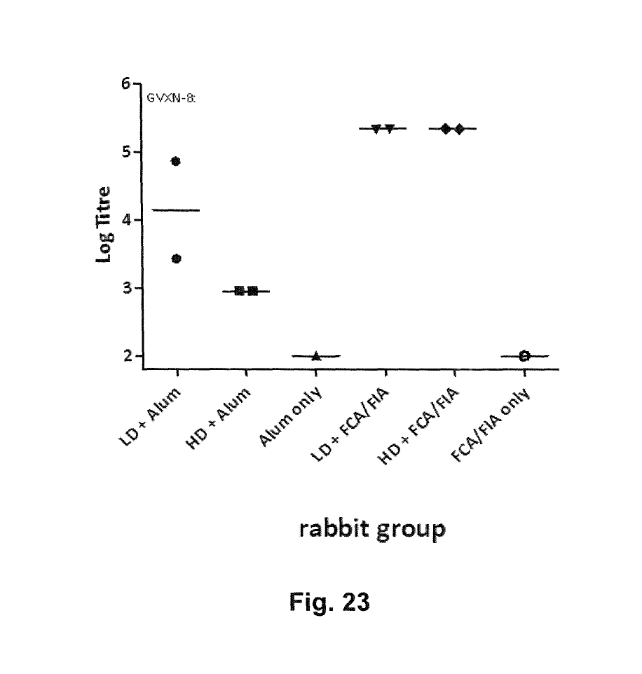

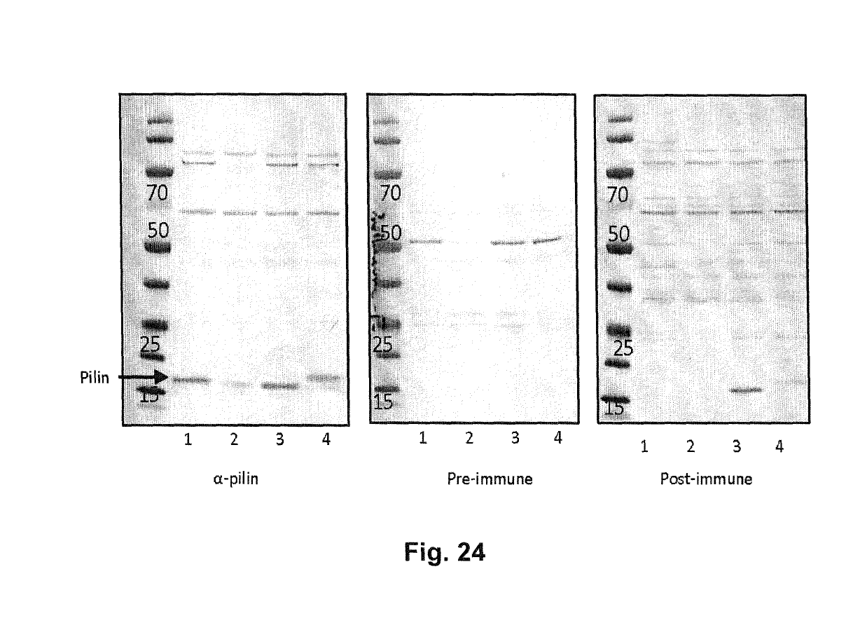

In another embodiment, the bioconjugates described herein can be used to generate antibodies for use in, e.g., diagnostic and research purposes. See Example 8 and FIGS. 23 and 24.

### 6. EXAMPLES

### 6.1 Example 1

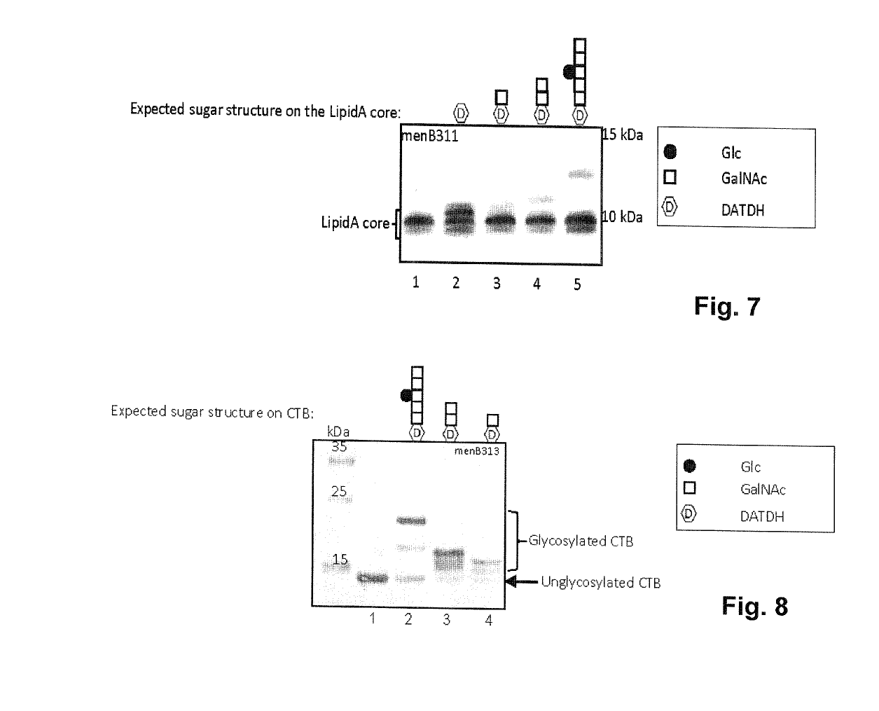

This example demonstrates that PglK, the flippase of C. jejuni, can flip short oligosaccharides into the periplasm of host cells. FIG. 7 shows that silver stained LOS variants were produced in E. coli SCM7, which lacks flippase activity. E. coli SCM7 was transformed with C. jejuni pgl plasmid variants lacking different glycosyltransferase activities but containing flippase (PglK) activity. Lanes 2-4 show bands with a lower mobility compared to lane 1, the lipid A core alone, with less mobility compared to C. jejuni Heptasaccharide on lipid A, lane 5. The upper band of lanes 2-4 corresponds to mono, di, and trisaccharides attached to the E. coli lipid A core. Thus, E. coli can utilize heterologous flippases to flip short sugar moieties into the periplasm.

### 6.2 Example 2

This example demonstrates that C. jejuni PglB can efficiently transfer di- and trisaccharides onto a protein acceptor in E. coli. FIG. 8 shows that PglB can transfer di- (lane 4) and tri-saccharides (lane 3) produced with C. jejuni pgl cluster variants, to engineered CTB (a carrier protein), containing two glycosylation sites. Thus, a heterologous oligosaccharyltransferase can be utilized in E. coli host cells to transfer short sugar moieties.

### 6.3 Example 3

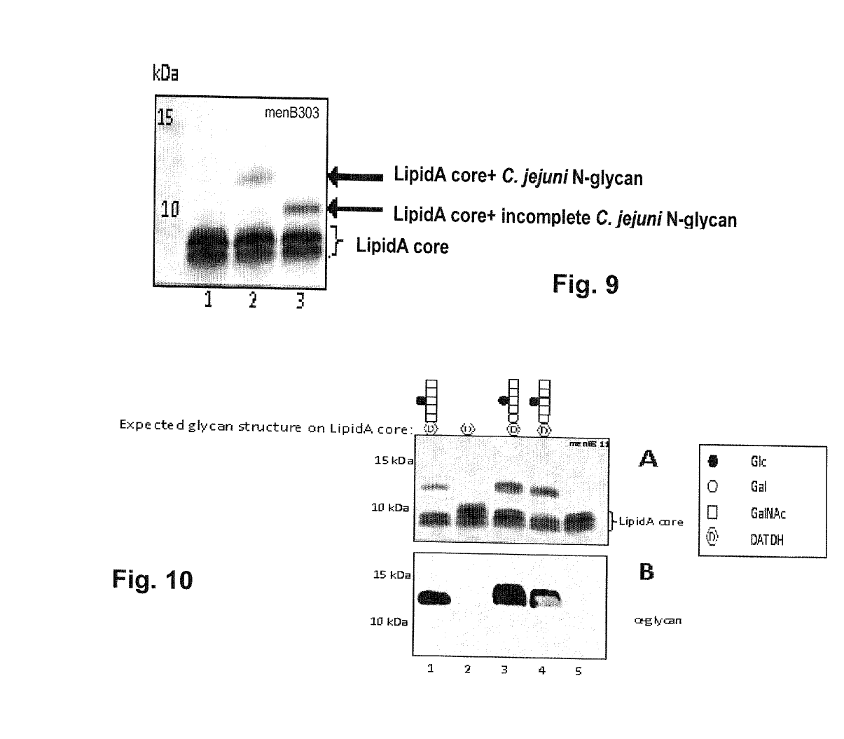

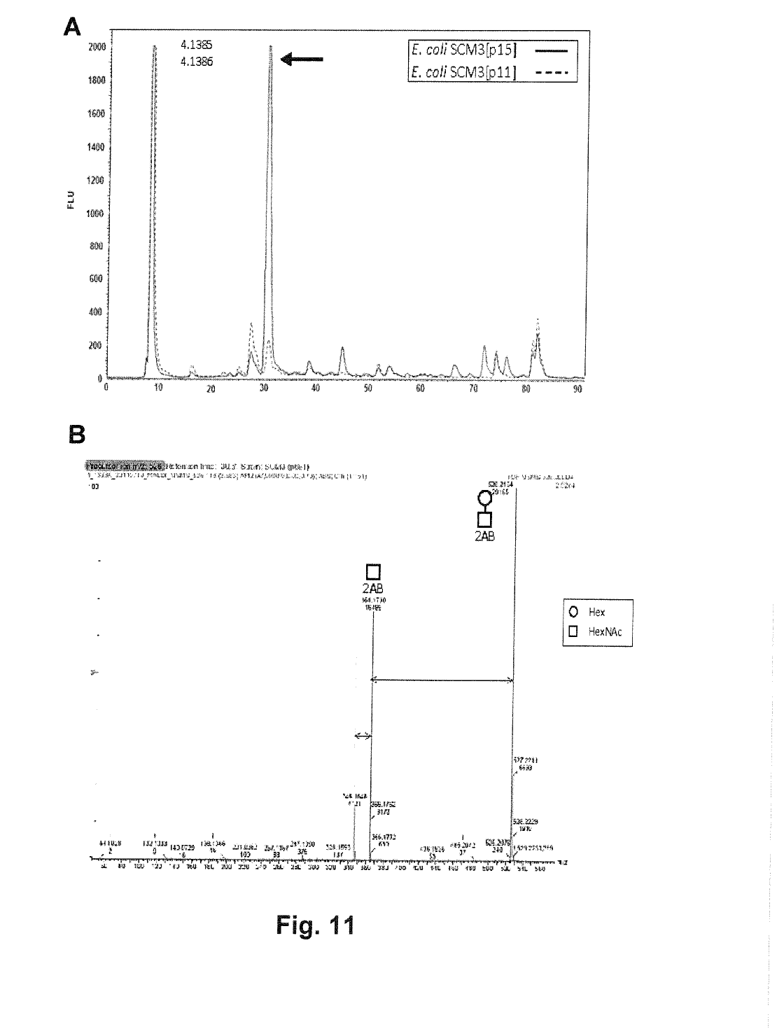

This example demonstrates that N. meningitidis flippase, PglF, has specificity toward short lipid-linked oligosaccharides. FIG. 9 shows silver stained lipooligosaccharide (LOS) produced in E. coli strain SCM7. It was demonstrated that when a plasmid comprising pglF nucleic acids was added in trans to E. coli SCM7 harboring a plasmid for expression of the C. jejuni pgl gene cluster lacking functional flippase (pglK), a band with a lower mobility shift (lane 2) as compared to the same strain bearing the wild type pgl cluster (lane 3) appears. The band that appears in lane 2 is demonstrated to have a higher mobility than the band corresponding to the lipid A core only that is produced by a strain containing the pgl cluster and lacking PglK flippase activity (lane 1).

### 6.4 Example 4

This example demonstrates that N. meningitidis PglA is functional in E. coli. E. coli strain SCM7 transformed with a C. jejuni pgl cluster could restore biosynthesis of a heptasaccharide by assembly of Gal on DATDH. FIG. 10 shows silver-staining and Western-blot analysis of LOS extracted from E. coli SCM7 transformed with a C. jejuni pgl cluster that has a mutation in PglA, which impairs assembly of the first GalNAc on the Undpp-DATDH in C. jejuni. Expression of N. meningitidis PglAI (FIG. 10A, lane 4) or Shigella dysenteriae O1 RfpB (FIG. 10A, lane 3), O antigen α-(1,3) Galactosyltransferase that assembles a Galactose residue on UndPP-GlcNAc, restore formation of LOS that is comparable in electrophoretic mobility shift with LOS formed in the strain bearing the intact C. jejuni gene cluster (FIG. 10A, lane 1). Also, expression of both S. dysenteriae RfpB (FIG. 10B, lane 3) and N. meningitidis PglA (FIG. 10B, lane 4) resulted in formation of the LOS variants that have comparable reactivity toward C. jejuni specific anti-glycan compared to LOS formed in the strain with the complete C. jejuni pgl gene cluster (FIG. 10B, lane 1). This reactivity was not observed when the same strain harboring the pgl gene cluster with a pglA mutation was transformed with an empty vector of galactosyltransferases (FIG. 10B, lane 2).

### 6.5 Example 5

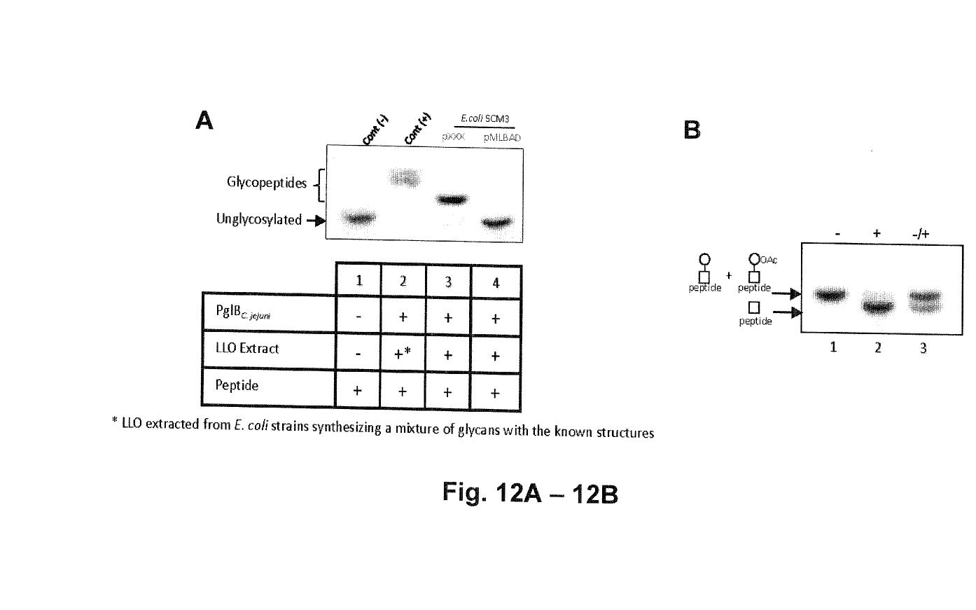

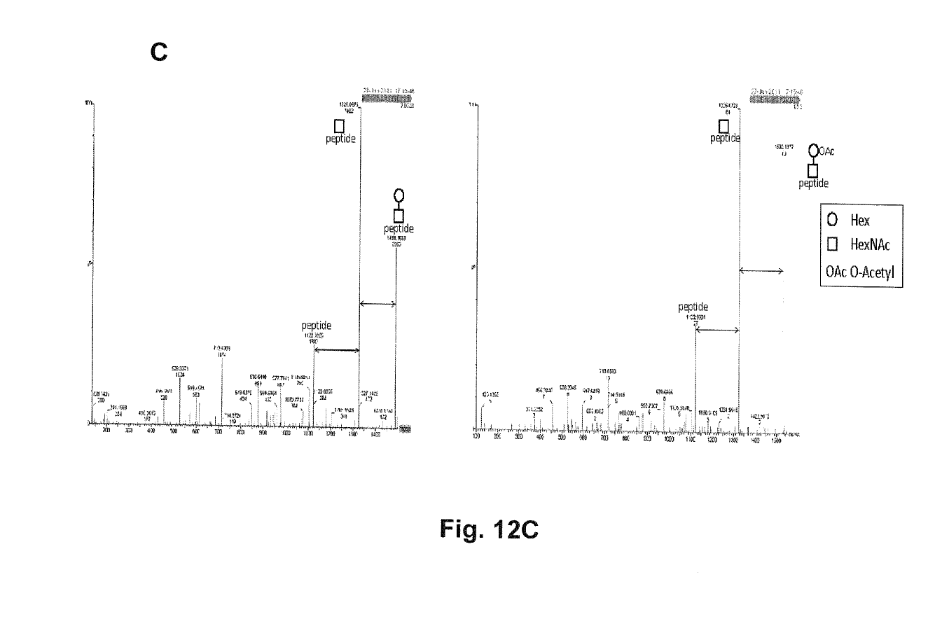

This example demonstrates that N. meningitidis PglA has α-(1,3) Galactosyltransferase activity in an E. coli background. The N. meningitidis pglA operon, containing Galactosyltransferase and O-acetyltransferase activities, was transformed into E. coli strain SCM3 that synthesizes UndPP-GlcNAc. The LLO was extracted from the strain and used for in vitro glycosylation using purified C. jejuni PglB, oligosaccharyltransferase, and a purified peptide acceptor. It is shown in FIG. 12 that when LLO from E. coli SCM3 expressing N. meningitidis pglAI was used for in vitro glycosylation the electrophoretic mobility of the peptide (FIG. 12A, lane 3) was reduced compared to unglycosylated peptide (FIG. 12A, lane 1). The purified peptide was subjected to MS/MS analysis, which demonstrated that the peptide had been glycosylated with Hex-HexNAc or Hex(OAc)-HexNAc (FIG. 12C).

The Glycopeptides were treated with α1-3,6 galactosidase from Xanthamonas manihotis and subjected to PAGE analysis. It was observed that the glycopeptide treated with Galactosidase (FIG. 12B, lane 2) has a lower mobility shift on gel compared to untreated sample (FIG. 12B, lane 1).

These results collectively indicate that Neisseria PglA functions as a Galactosyltransferase in E. coli and that this enzyme possesses a relaxed specificity toward acceptor glycan, which allows using both UndPP-GlcNAc and Ump-DATDH. Furthermore, it is demonstrated that PglI, an O-acetyltransferase, is functional in E. coli.

### 6.6 Example 6

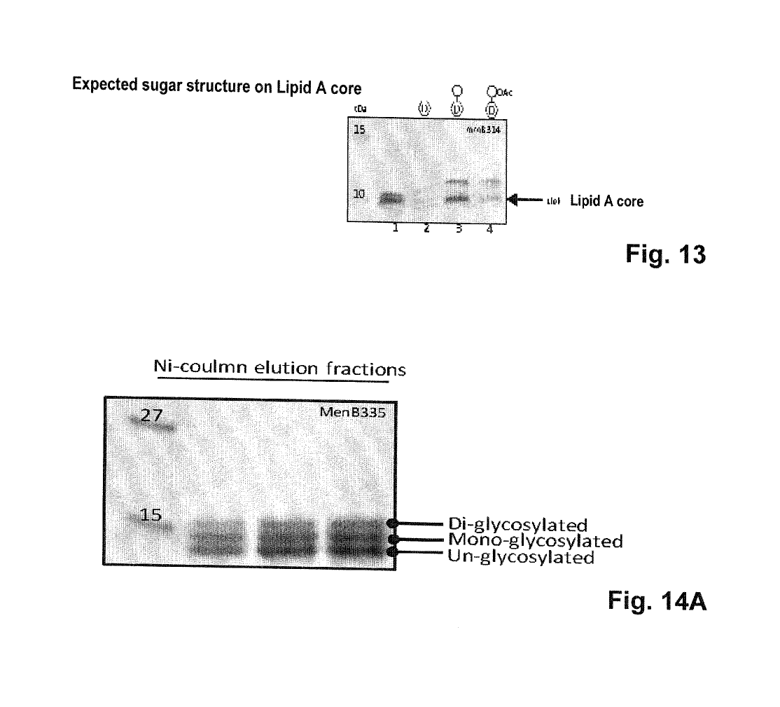

This example demonstrates the synthesis of N. meningitidis disaccharide, Gal-DATDH, in E. coli. Production of the disaccharide was achieved using the C. jejuni pgl cluster carrying a transposon mutation in galE, an epimerase. Mutation of this epimerase abrogates production of UDP-GalNAc and therefore results in accumulation of Undpp-DATDH. FIG. 13 shows silver-stained LOS produced in E, coli strain SCM7 that was transformed with a C. jejuni pgl galE mutant and an N. meningitidis pglAI (lane 4) or S. dysenteriae rfpB (lane 3). As shown, bands with lower electrophoretic mobility were observed than LOS from the strain that harbors a corresponding empty vector (lane 2). These results indicate that both PglA and RfpB can interchangeably be used to form Gal-DATDH. It also indicates that RfpB can accept UndPP-DATDH as an acceptor.

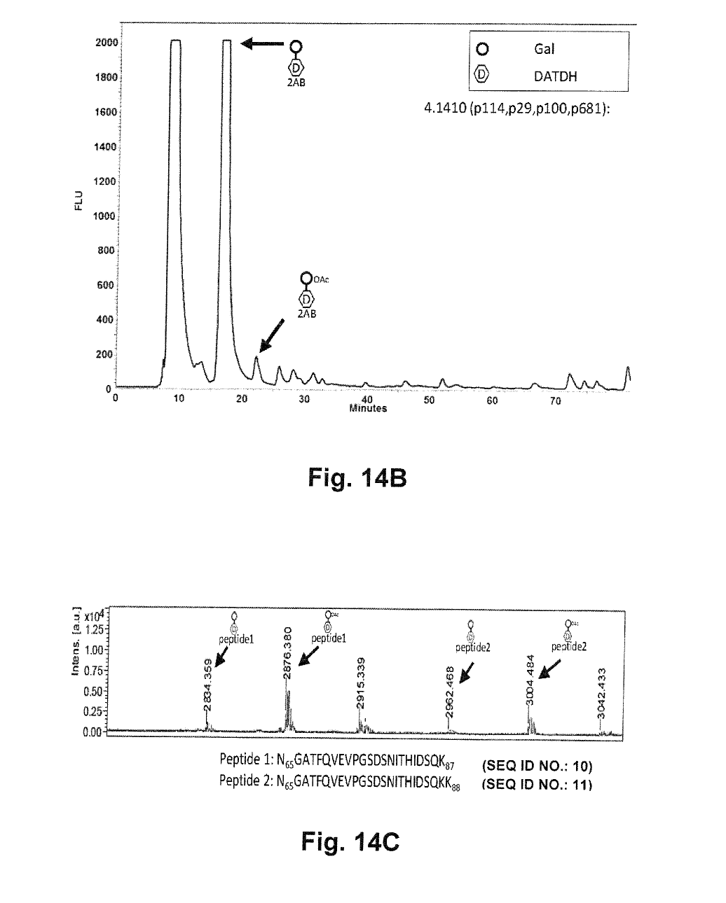

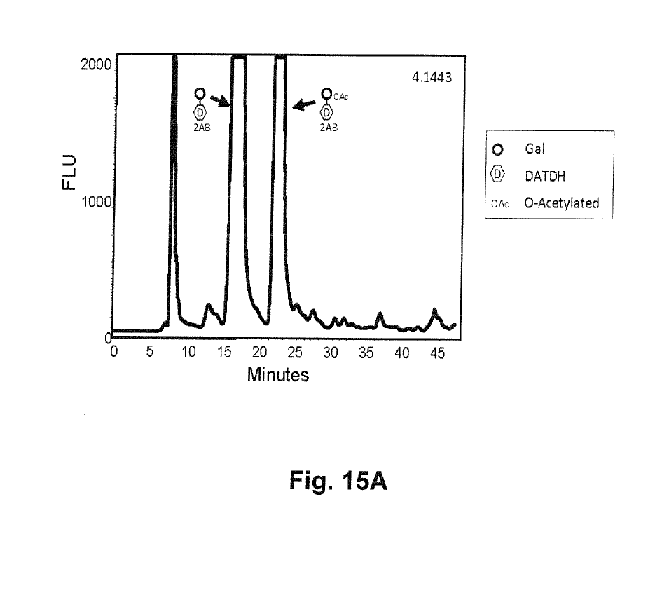

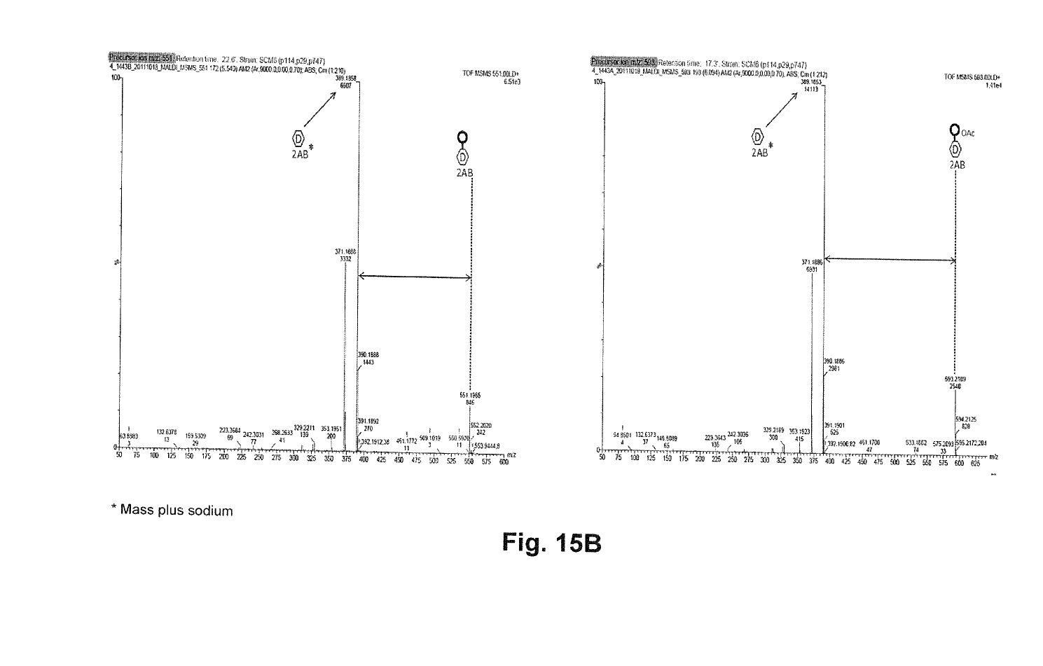

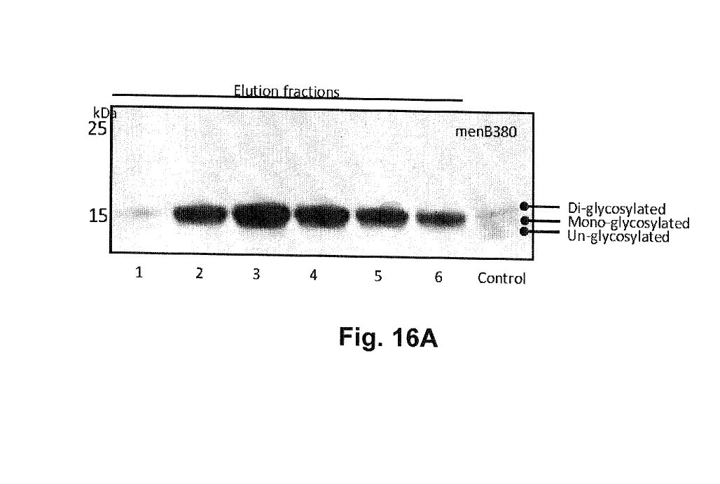

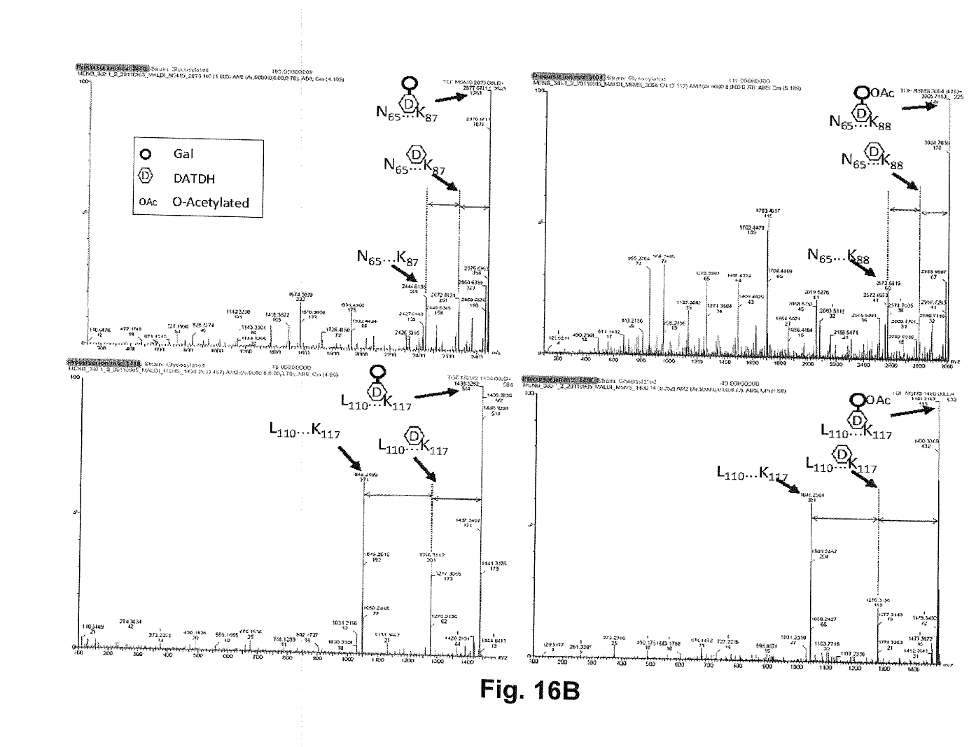

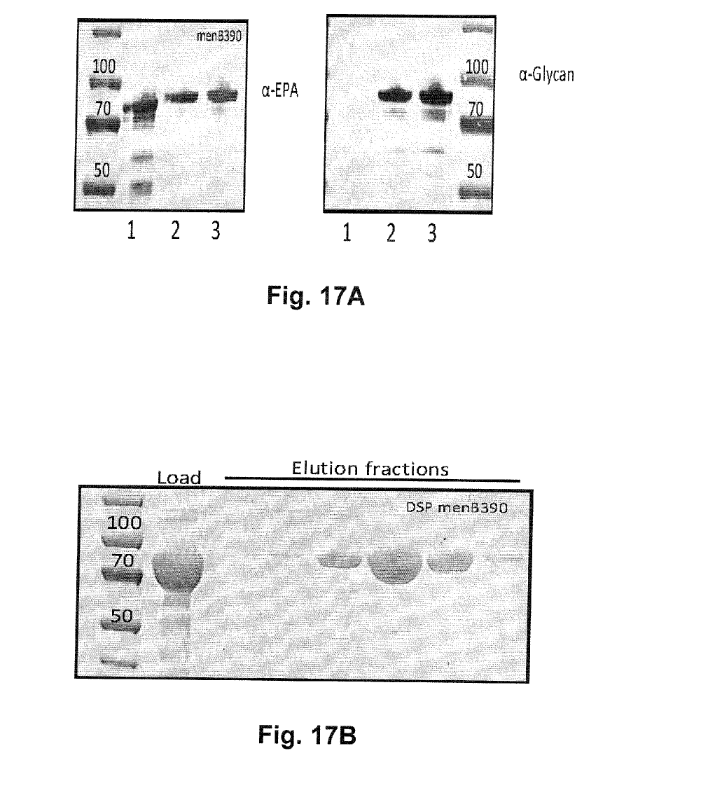

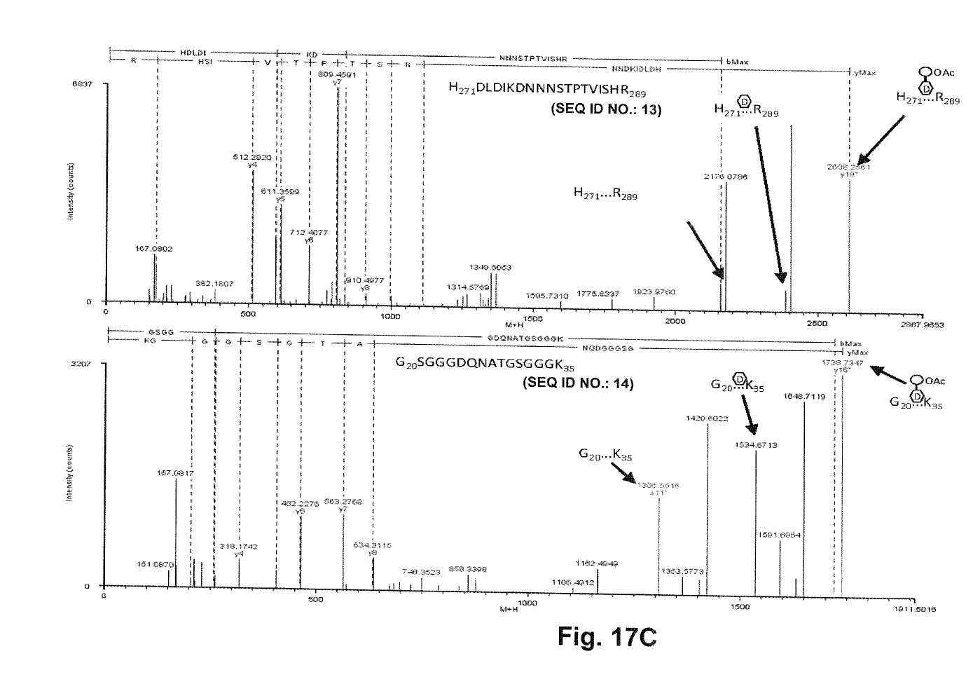

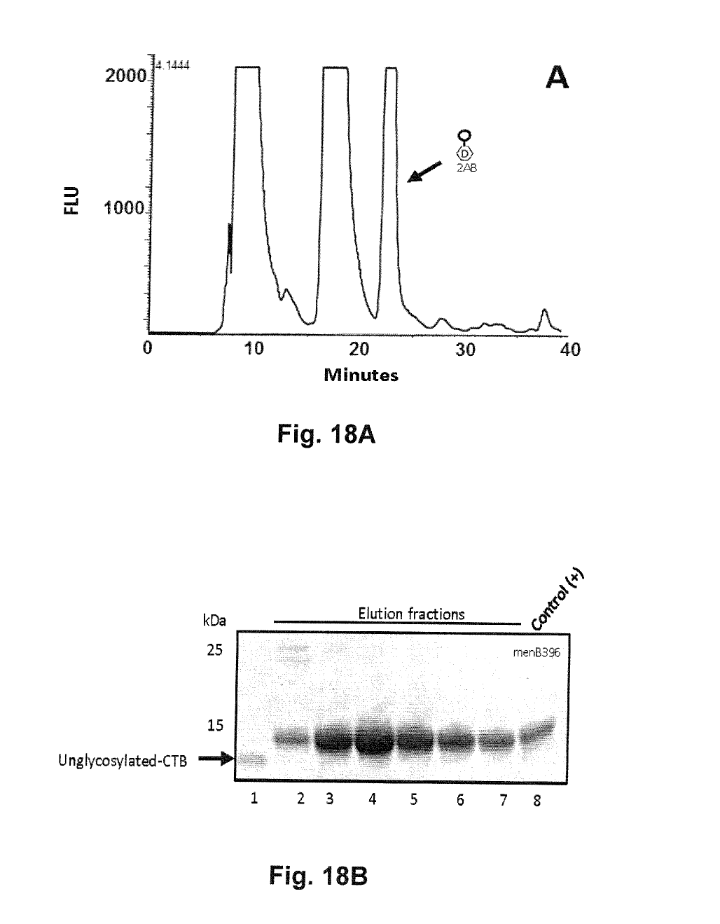

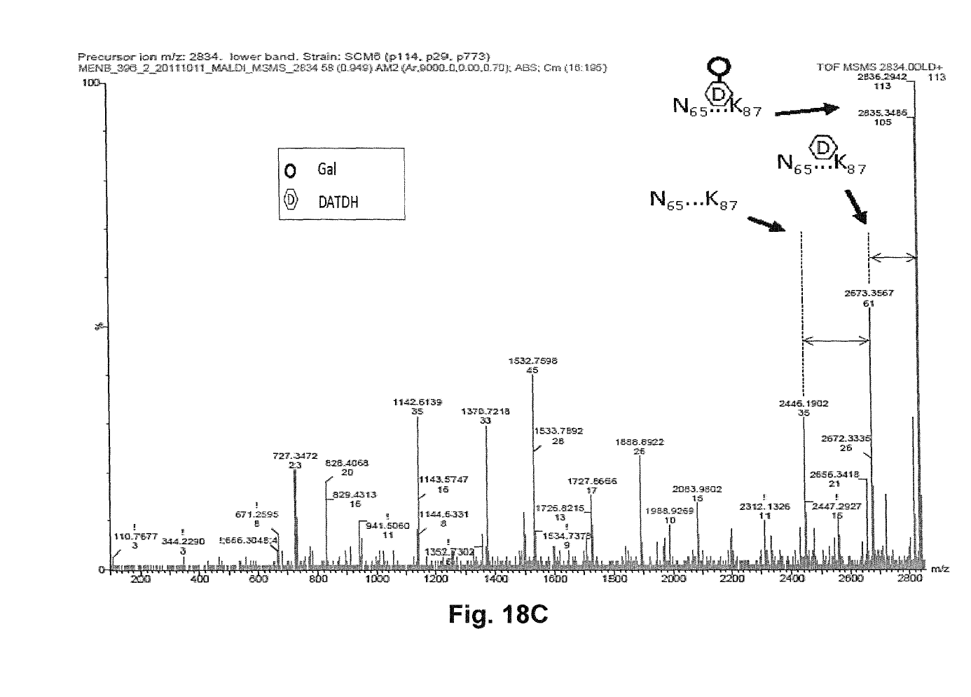

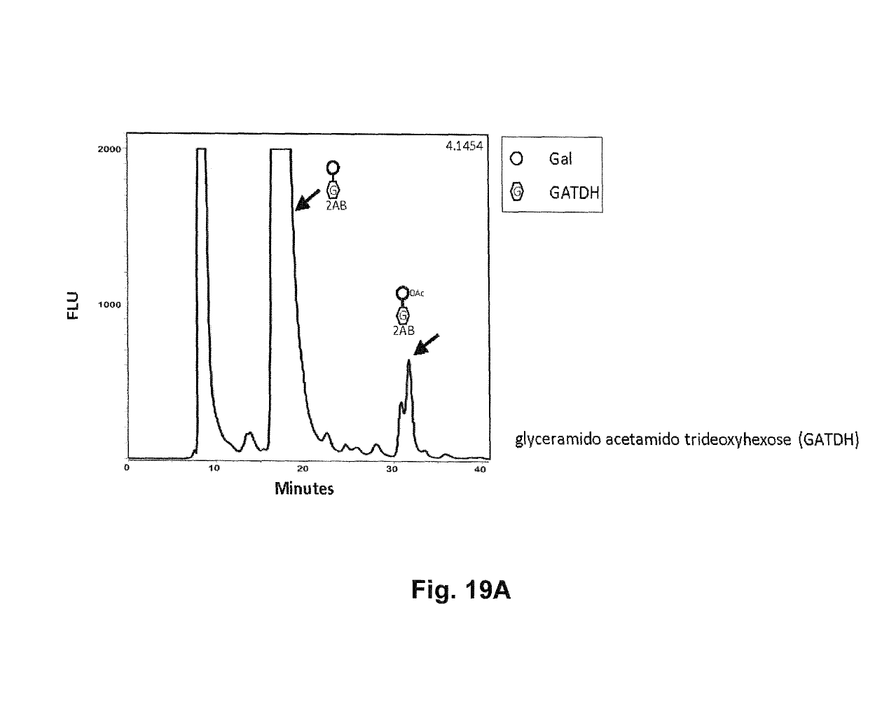

This example also demonstrates the synthesis of a glycoconjugate with N. meningitidis disaccharide, Gal(OAc)-DATDH. Glycosylation of CTB with a disaccharide was achieved in E. coli strain SCM6 by combining plasmids for expressing C. jejuni pgl galE mutant, N. meningitidis PglAI, CTB and C. jejuni pglB. FIG. 14 shows PAGE analysis and MS analysis of glycosylated CTB with a disaccharide Hex-DATDH, which Hex could also be found in acetylated form. Furthermore, it was demonstrated that by using a plasmid containing an N. meningitidis synthetic cluster, pglFBCDAI, in E. coli strain SCM6, a large quantity of UndPP-Hex(OAc)-DATDH was produced (FIG. 15A). FIG. 16 shows that CTB was efficiently glycosylated in vivo with the disaccharide compared to previously described system using combination of genes from different bacteria (FIG. 15A, control). MS analysis of glycopeptide released from glycosylated CTB demonstrated the sequence of disaccharide to be Hex_DATDH or Hex(OAc)-DATDH. FIG. 17 demonstrates that engineered EPA containing 4 glycosylation sites can be glycosylated with the disaccharide in an E. coli background. FIG. 18 shows the disaccharide without an O-acetyl group, Gal-DATDH, can also efficiently be transferred to CTB in an E. coli background.

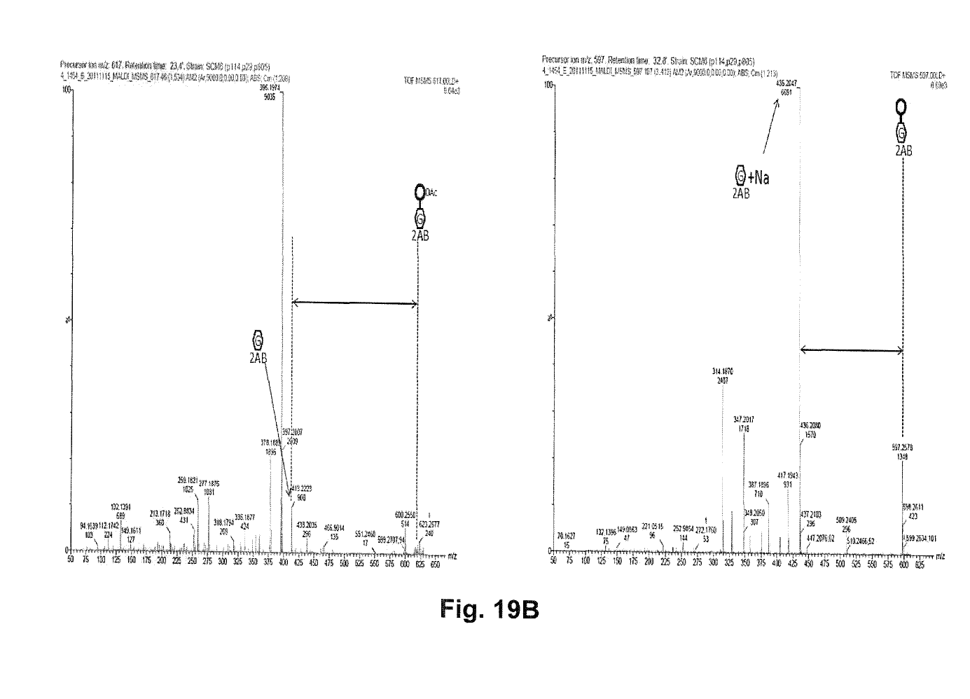

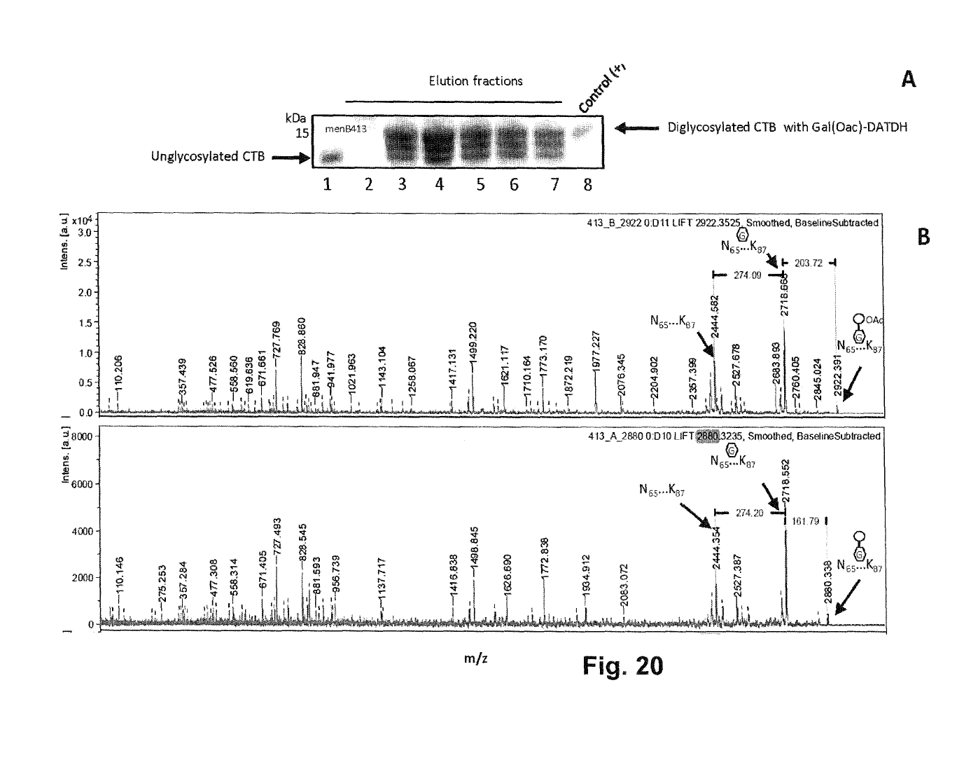

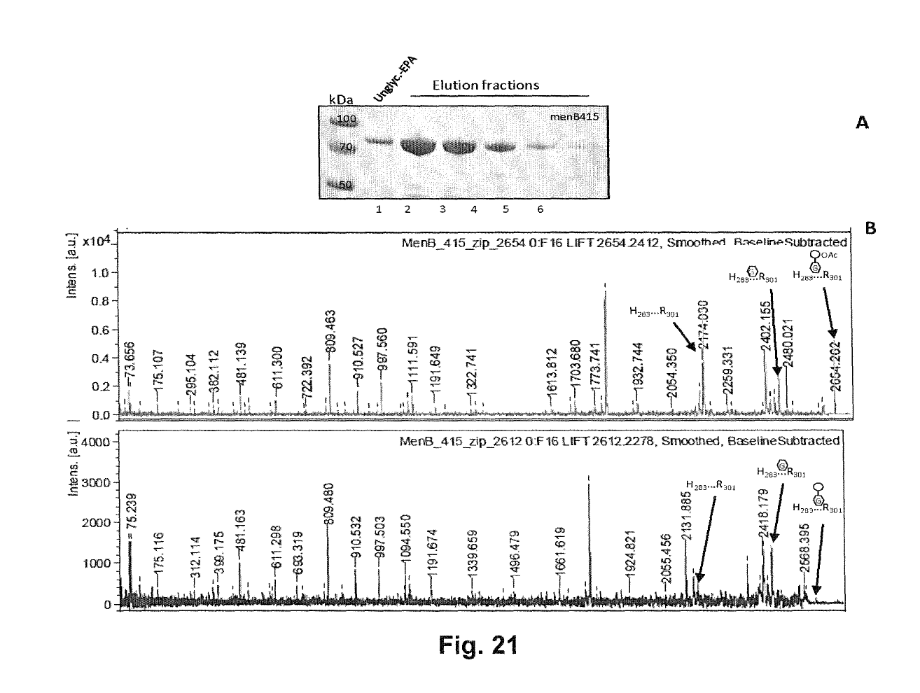

This example also demonstrates the synthesis of a glycoconjugate with N. meningitidis disaccharide, Gal(OAc)-GATDH. Glycosylation of CTB with disaccharide was achieved in E. coli strain SCM6 by combining plasmids for expressing N. meningitidis synthetic cluster pglFB2CDAI in E. coli SCM6, CTB and C. jejuni pglB. FIG. 20 shows PAGE analysis (FIG. 20A) and MS analysis of glycosylated CTB (FIG. 20B) with a disaccharide Flex-GATDH or Hex(OAc)-GATDH. FIG. 21 shows glycosylation of EPA with Gal(OAc)-GATDH.

### 6.7 Example 7

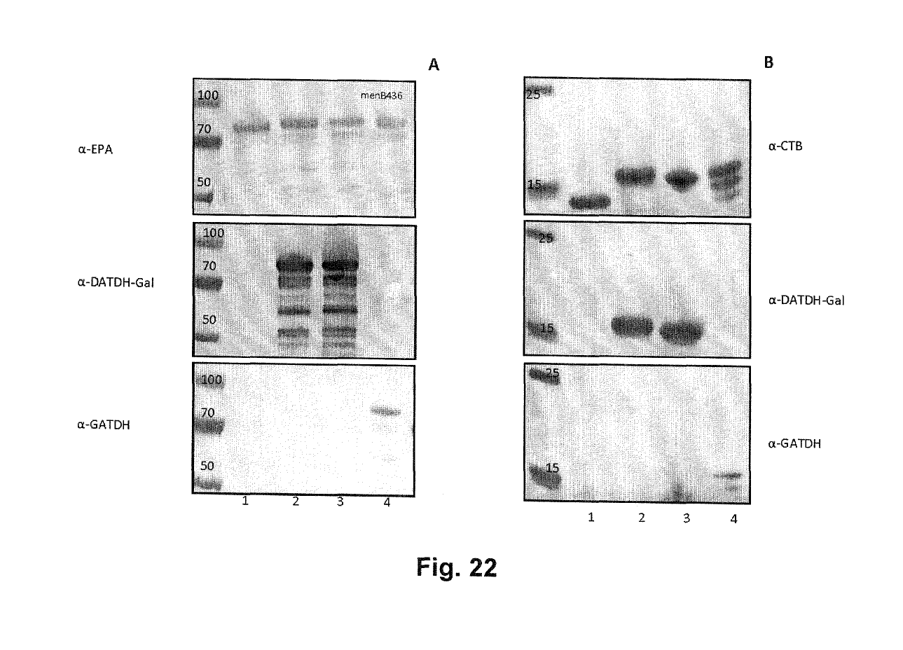

This example demonstrates that specific glycan antibodies are raised against O-glycosylated pilin from different Neisseria strains and that such antibodies recognize recombinant N-glycosylated proteins having identical glycan structures. See FIG. 22.

### 6.8 Example 8

This example demonstrates that recombinant glycosylated CTB with Gal-DATDH is immunogenic in rabbits. See FIGS. 23 and 24.

### 6.9 Example 9

This example demonstrates that disaccharides can be successfully transferred onto protein carriers, specifically cholera toxin B (CTB), using the C. jejuni glycosylation machinery in an E. coli background.

(a) Materials and Methods

(i) Bacterial Strains, Plasmids, Media and Cultures

Escherichia coli strains, as well as the plasmids used in this example are listed in Table 1. Generally, cultures were grown in LB at 37° C., supplemented by ampicillin (100 μg/mL), chloramphenicol (30 μg/mL), kanamycin (50 μg/mL), spectinomycin (80 μg/mL), or trimetoprim (100 μg/mL) when required. If indicated, strains were grown in Terrific Broth (TB, 12 g Bacto Tryptone, 24 g Bacto Yeast Extract, 4 mL Glycerol and 100 mL 0.17M KH2PO4 and 0.72M K2HPO4, for 1 L), Super Optimal Broth (SOB, 20 g Bacto Tryptone, 5 g Bacto Yeast Extract, 10 mM NaCl, 2.5 mM KCl, 10 mM MgCl2, for 1 L), Tryptic Soy Broth (TSB, BD), or Brain Heart Infusion (BHI, Merck). When indicated, 10 mM MgCl₂ was added to the cultures.

TABLE 1

| Strain/ | | | Selection |
|---|---|---|---|
| Plasmid | Characteristics | Induction | Marker |
| SCM6 | Sψ801acZΔM15 Δ(lacZYA-argF) | | |
| | U169 recA1 endA1 hsdR17 (rk−, | | |
| | mk+) phoA supE44 λ-thi-1 gyrA96 | | |
| | relA1 | | |
| Plasmid 2 | pEC415_CTB 2 sites of | Arabinose | AmpR |
| | glycosylation | | |
| Plasmid 3 | Pgl cluster, pglJ::Kan | Consti- | ClmR, |
| | | tutive | KanR |
| Plasmid 4 | Pgl cluster, pglH::Kan | Consti- | ClmR, |
| | | tutive | KanR |
| Plasmid 5 | pglB in pEXT21 | IPTG | SpcR |
| Plasmid 12 | pglA_I N. meningitidis in | Arabinose | TmpR |
| | pMLBAD | | |
| Plasmid 16 | pglFBCDAI N. meningitidis in | Arabinose | TmpR |
| | pMLBAD, pglF in the wrong | | |
| | orientation | | |
| Plasmid 17 | pglFBCDAI N. meningitidis in | Arabinose | TmpR |
| | pMLBAD, pglF in the right | | |
| | orientation | | |

(ii) Molecular Biology

PCR and colony PCR reactions were performed using Taq Polymerase (˜1 kbp/min), 25 mM MgCl₂, 10 mM dNTP (Fermentas), 5 μM of primer, and DNA (or one colony directly from a plate) as template. For high fidelity PCR, Phusion enzyme was used (1U, Finnzyme).

Restriction analyses were performed using 5-50 U of enzymes (Fermentas) in their buffer of highest activity. Approximately 2 μg of DNA was digested for 1-2 h at 37° C. Enzymes were then heat-inactivated for 20 min at 65° C. Enzymatic digestions were ran on a 0.7% agarose gel, where DNA was visualized using GelRed (Biotum). The migration was performed in Tris, Acetate, and EDTA (TAE) buffer.

For cloning, the insert and the vector were both digested as described above. The digested vector was then incubated for 1 h at 37° C. with 10 U Shrimp Alkaline Phosphatase (SAP, Fermentas) for dephosphorylation. After inactivation, digested products were cleaned using the NucleoSpin Extract II kit (Macherey-Nagel) following the manufacturer's protocol. One hundred ng of vector and 100-300 ng of insert were incubated for 2 h at room-temperature in the presence of 5 U T4 DNA ligase. The enzyme was then inactivated and competent DH5α were transformed using 20 ng of the ligation by heat-shock (1 min at 42° C. and 2 min on ice).

The sequence of each of the created plasmids was verified by DNA sequencing (MicroSynth).

(iii) Cell Growth

For protein and sugar expression, cells were diluted from an overnight (ON) preculture grown at 30-37° C. to an OD₆₀₀ nm of 0.05-0.1. Cells were grown at 37° C. until 0.4-1 and induced using 0.02-0.1% arabinose (w/v), and 1 mM IPTG when needed. Cells were incubated at 37° C. for 4 hours or for the night. Whole Cell Extracts (WCE) or periplasmic extractions were performed. For the former, cells were harvested (5000×g, 15 min, 4° C.), resuspended at 1 OD/100 μL in Lämmli buffer and boiled for 15 min at 95° C. The latter consisted of the incubation, after harvesting, for 30 min at 4° C. in Lysis Buffer containing 1 mg/mL Lysozyme (Lysis Buffer: 20% w/v sucrose, 30 mM Tris-HCl pH 8, 1 mM EDTA, Lysis buffer is used at 20 OD/mL). The suspension was centrifuged at 23,000×g for clarification and the periplasmic fraction corresponded to the supernatant. If MgCl₂ was added to the culture, the lysis step was preceded by a washing step in 30 mM Tris-HCl pH 8, 1 mM EDTA.

For the purification of the His-tagged proteins, Binding Buffer (0.5 M NaCl, 50 mM Tris-HCl pH 8, 10 mM Imidazole) and MgCl₂ to a final concentration of 4 mM were added to the periplasmic fractions. The preparation was loaded on a His SpinTrap (GE Healthcare) or HisTrap FF crude 1 mL or 5 mL (GE Healthcwere) and in PBS, 500 mM Imidazole, adjusted to pH 7 for CTB.

After purification, protein concentration was determined by NanoDrop (NanoDrop 2000C, Thermo Scientific) and by BCA, following the provided protocol.

(iv) Protein Analysis

For the analysis of the glycosylated proteins by Coomassie staining, purified proteins were loaded after boiling 15 min in Lämmli Buffer on a SDS-PAGE gel and run at 200 V for 50 min for 70 min in MES buffer for CTB. After several washes in water, proteins were stained using Coomassie Simply Blue.

For Western-Blot analysis, after the separation on an SDS-PAGE gel, proteins were transferred on a nitrocellulose membrane using a semi-dry transfer (iBlot, Invitrogen). The membrane was blocked for 1 h in PBS-Tween 20 at 0.1%+10% Milk. After several washes in PBS-Tween, the membrane was incubated with the primary antibody, diluted in PBS-Tween+10% Milk, for 1-2 h at room-temperature or 4° C. over night. After a washing step in PBS-Tween, the secondary antibody coupled to Horse Radish Peroxidase (HRP) was added for 1 h at room-temperature or over night at 4° C. The membrane was washed again and proteins were visualized by the addition of TetraMethylBenzidine (TMB, Sigma). The antibodies used included: (a) primary antibodies: mouse anti-Penta-His (Qiagen, diluted 1:2000); rabbit anti-Cholera toxin (Virostat, diluted 1:1000); rabbit antiserum R12 (Pineda, diluted 1:2000); (b) secondary antibodies: goat anti-mouse HRP (Sigma, diluted 1:2000); and goat anti-rabbit HRP (Bio-Rad, diluted 1:20,000).

(b) Results

(i) Transfer of the Pilin Glycan onto the CTB

It has been previously shown that the C. jejuni N-glycosylation machinery, once transferred to E. coli, has a relaxed specificity towards the carbohydrate added to the target protein. The carbohydrate tested here was a disaccharide and no previous study had been performed concerning the transfer of such a short sugar with the pgl machinery. Thereby, whether a disaccharide could be transferred to the protein carrier CTB, which possesses two sites of glycosylation, was investigated.

(A) Glycosylation of CTB by C. jejuni Disaccharide

In C. jejuni, N-glycosylation comprises the addition of a heptasaccharide (GalNAc-α1,4-GalNAc-α1,4-(Glc-β1,3)-GalNAc-α1,4-GalNAc-α1,4-GalNAc-α1,3-Bac) to a lipid carrier, undecaprenylpyrophosphate. This heptasaccharide is then transferred to an asparagine residue within the specific and conserved sequence Asp/Glu-X₁-Asn-X₂-Ser/Thr of a protein (where X₁ and X₂ are any amino acid except proline), and occurs in the periplasm. More specifically, in C. jejuni, the reaction begins with the sequential conversion of a uridine diphosphate UDP-GlcNAc or UDP-GalNAc in UDP-Bac (Bacillosamine, 2,4-diacetamido-2,4,6-trideoxyglucose) by a dehydratase (PglF), an aminotransferase (PglE) and an acetyltransferase (PglD). This UDP-Bac is then transferred to the lipid carrier undecaprenyl phosphate (Und-P) by PglC, in order to create the first intermediate undecaprenylpyrophosphate-bacillosamine (Und-PP-Bac), on the cytoplasmic side of the inner membrane. A first GalNAc residue is added by PglA, and a second is added by PglJ, before PglH adds 3 other GalNAc residues. Finally, a unique Glc residue, branched in position 4, is linked by PglI. Once the entire heptasaccharide is formed, it is flipped into the periplasm by PglK, before being transferred onto the Asn residue of an acceptor protein by PglB.

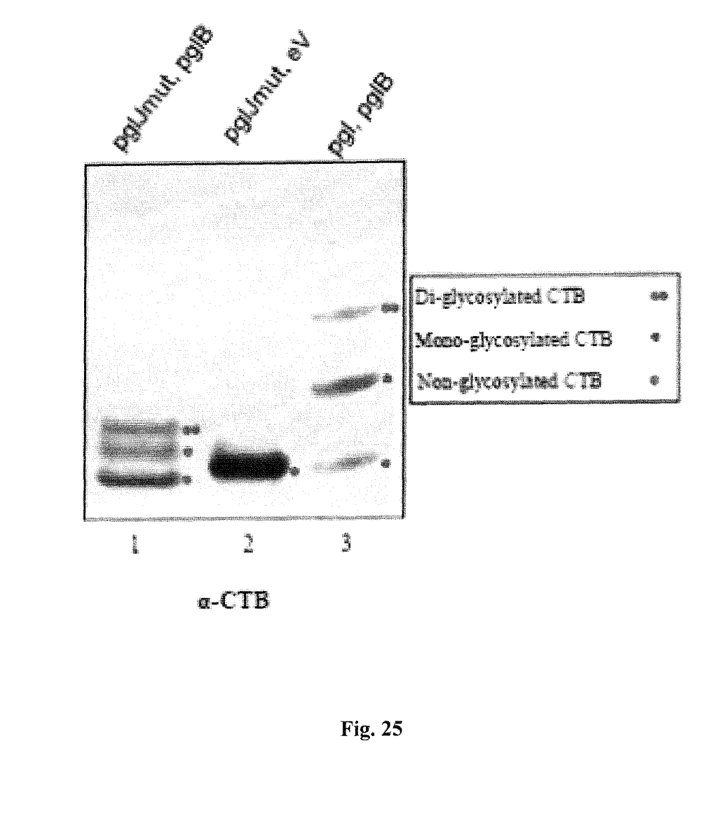

The pglJ gene, which catalyzes the addition of the third GalNAc residue of the heptasaccharide of C. jejuni, has been previously mutated inside the pgl cluster, which results in the formation of a disaccharide Bac-GalNAc on the Und-PP. To test whether PglB can N-glycosylate the CTB with such a short sugar, this mutated cluster, expressed by plasmid 3 (see Table 1), the gene of the protein carrier CTB presenting 2 glycosites, expressed by plasmid 2, and plasmid 5, which results in the overexpression of PglB, were introduced into the E. coli strain SCM6. This strain does not express any endogenous sugar on its surface and allows for the study of the carbohydrate of interest. Following 4 h of induction with 0.1% of arabinose and 1 mM of IPTG, the periplasm of these cells was extracted according to the method described in above, and the glycosylation state of the CTB was analyzed by Western-Blot. As shown in FIG. 25, the control of the glycosylation by the heptasaccharide of C. jejuni results in a variation of the mobility of the CTB on SDS-PAGE gel (FIG. 25, lane 3). Three bands can be distinguished in lane 3 of FIG. 25—the highest mobility band corresponds to the unglycosylated form of the CTB; the intermediate mobility band corresponds to the glycosylation of the CTB by the heptasaccharide, on only one of its two sites; and the lower mobility represents the CTB glycosylated on each of its two sites. This mobility shift was also observed in the context of the mutation of pglJ, but here, the shift observed was shorter (FIG. 25, lane 1). Three bands were also observed in lane 1 of FIG. 25. By analogy, these bands correspond to the un-, mono- and di-glycosylated form of the CTB. When PglB is not overexpressed, only the non-glycosylated form of the CTB is detected (FIG. 25, lane 2). This mobility shift observed between the non-mutated and mutated form of pglJ indicates that the CTB is glycosylated by shorter carbohydrates than the heptasaccharide of C. jejuni.

These data indicate that the mutation of PglJ inside the pgl cluster results in the formation of a disaccharide, and this disaccharide can be transferred onto the protein carrier CTB, using the C. jejuni glycosylation machinery.

(B) Glycosylation by the N. meningitidis Disaccharide and its Optimization

As shown above, PglB can transfer a disaccharide to CTB. In the context of the mutation of the epimerase GalE, which catalyzes the transformation of UDP-GlcNAc into UDP-GalNAc, the transfer of the bacillosamine of C. jejuni elongated by the O-acetylated galactose of Neisseria meningitidis is possible. The mutation of the epimerase GalE of C. jejuni prevents the addition of GalNAc residues on the bacillosamine. However, a weak expression of the CTB and glycosylation level by this new disaccharide was observed. In order to improve it, different combinations of antibiotics were tested to decrease the stress undergone by the cells. Several culture media were also tested, as well as the addition of MgCl2.

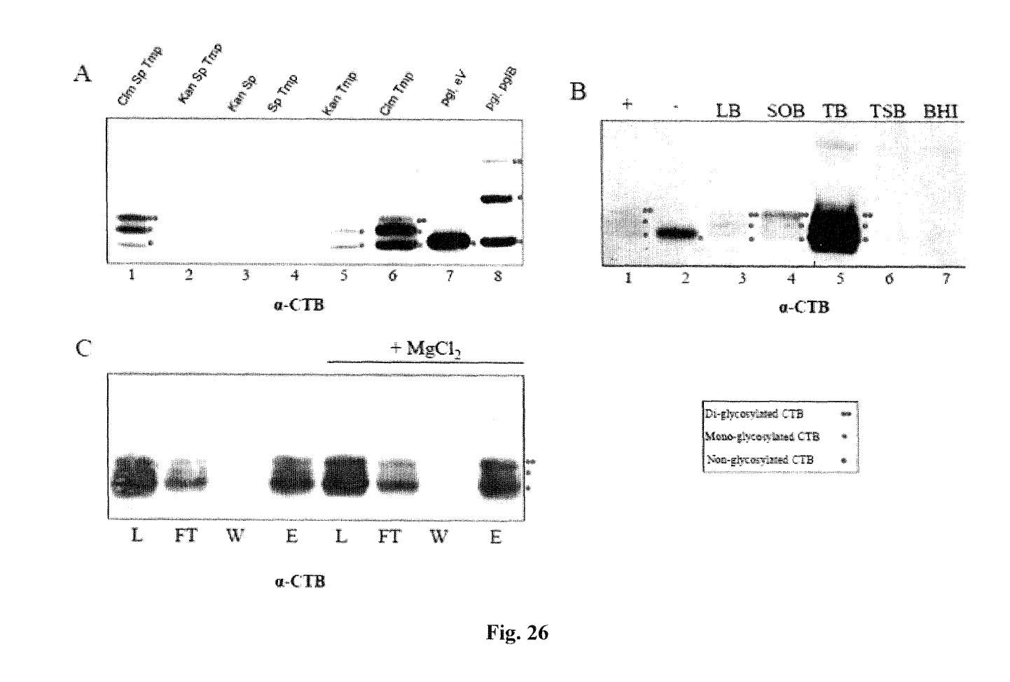

Plasmid 4, carrying the C. jejuni glycosylation cluster mutated for GalE, as well as plasmids 2, 5 and 12, allowing the elongation of the bacillosamine of C. jejuni by the O-acetylated galactose of N. meningitidis, were introduced into the E. coli strain SCM6. When this strain was grown in the presence of the 4 markers of selection carried by the plasmids (respectively Clm/Kan, Amp, Spc, and Tmp), no expression of the CTB was detected in the periplasm of the cells after 4 h of induction or ON. To remedy this, several combinations of antibiotics were tested. Periplasmic fractions were collected after 4 h of induction and analyzed by Western-Blot. As shown in FIG. 26A, the level of expression of the CTB and its glycosylation were highly influenced by the antibiotics. For instance, the addition of kanamycin (FIG. 26A, lanes 2 and 3) seems to strongly impair the expression of the CTB. The highest level of expression and glycosylation of the CTB was in the presence of chloramphenicol, spectinomycin and trimetoprim (FIG. 26A, lane 1). Therefore, this combination was further analyzed.

Next, different culture media were tested, with the aim to improve the expression and the glycosylation of CTB. The same strain of SCM6 carrying plasmids 2, 4, 5, and 12 was grown in LB, SOB, TB, TSB, or BHI. FIG. 26B represents the analysis of the periplasmic fractions of the cells, induced ON, by Western-Blot. Whereas the enriched media TSB and BHI (FIG. 26B, lanes 6 and 7) do not show any expression of the CTB, expression and glycosylation were strong in TB medium (FIG. 26B, lane 5). Weak expression and glycosylation were observed in LB, as seen lane 3 of FIG. 26B. Finally, SOB medium containing 10 mM of MgCl2 showed a higher ratio of di-glycosylated form of the CTB than when MgCl2 was absent. TB medium, which allowed for the highest level of expression of the CTB was therefore used for the next experiments.

In order to assess more accurately its level of glycosylation, CTB was purified using its histidine tag on a Ni²⁺ column, from the periplasmic extracts of cells induced ON, and grown in TB with or without 10 mM MgCl₂. The analysis of each step of the purification of the CTB grown in presence or not of MgCl₂, was performed by Western-Blot. As shown in FIG. 26C, the majority of the CTB could be purified. Only a low amount of the protein was found in the Flow Through (FT). The elution fraction of the CTB, where the cells were grown in TB+10 mM MgCl₂ shows a slightly higher level of diglycosylation, compared to that in cells grown without MgCl₂. Thus, it appears that MgCl₂ has a positive effect on the glycosylation of the CTB.

As seen, the transfer of a disaccharide on the CTB is possible using the glycosylation machinery of C. jejuni. Therefore, this was used for the transfer of the O-acetylated disaccharide of N. meningitidis to CTB. The levels of expression and of glycosylation were been optimized, and the best conditions of culture appear to be in TB, in presence of chloramphenicol, spectinomycin and trimetoprim. Further, the addition of 10 mM MgCl₂ has a positive effect on the glycosylation of the CTB.

(C) Large Scale Expression and Purification of the Glycosylated CTB

In order to collect large amounts of glycosylated CTB, its purification from a higher volume of culture was performed using the conditions described above.

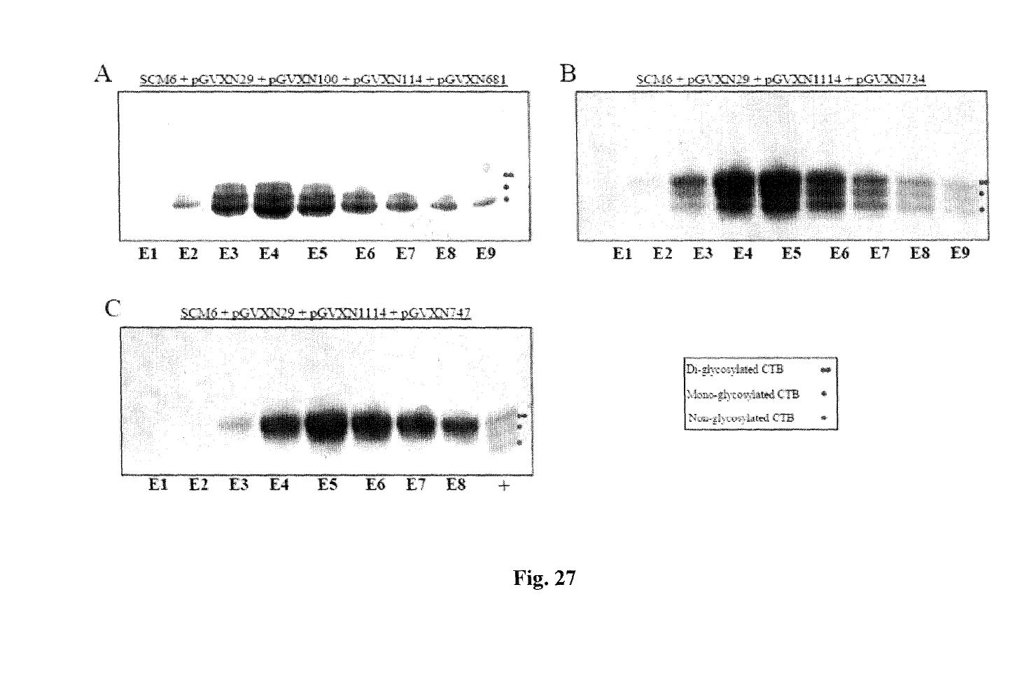

Plasmids 2, 4, 5 and 12 were introduced into the E. coli strain SCM6 and grown in 6 L of TB supplemented with chloramphenicol, spectinomycin, trimetoprim and 10 mM MgCl2. The extraction of the periplasmic fractions was performed and the CTB was purified, as indicated in the Materials and Methods section above. FIG. 27A depicts the different elution fractions collected during the purification on a 5 mL HisTrap column, and analyzed on SDS-PAGE gel stained by Coomassie Simply Blue. The un-, mono- and di-glycosylated forms of the CTB were detected in elution fractions E1 to E9.

Plasmid 16 bears all the genes necessary for the assembly and the translocation to the periplasm of the entire O-acetylated disaccharide of N. meningitidis, but this operon contains the flippase pglF in the wrong orientation. This allows, on one hand, for the study of the biosynthesis of the unmodified disaccharide of N. meningitidis (without using the bacillosamine of C. jejuni), and on the other hand, allows for the replacement of plasmids 4 and 12 by the single plasmid 16. This plasmid was tested, and the un-, mono- and di-glycosylated forms of the CTB were detected, in TB supplemented by ampicillin, spectinomycin, trimetoprim and 10 mM MgCl2. This plasmid was introduced into the E. coli SCM6 strain, along with plasmids 2 and 5. The cultures were grown in 6 L TB, with ampicillin, spectinomycin, trimetoprim and 10 mM MgCl2 added. The periplasmic fractions were extracted as described, purified on a Ni²⁺ column, and analyzed by SDS-PAGE gel stained by Coomassie Simply Blue (FIG. 27B). Although the presence of aggregates was detected in the first elution fraction (E1), a high amount of CTB, mainly glycosylated (mono- and di-glycosylation) was collected (lanes E2 to E9). A BCA assay was performed, indicating that approximately 14 mg of CTB was collected.

Finally, plasmid 17 contains the entire operon for the synthesis of the disaccharide of N. meningitidis, but in which the flippase pglF is in the correct orientation. When introduced into E. coli SCM6 with plasmids 2 and 5 and grown in TB supplemented with ampicillin, spectinomycin, trimetoprim and 10 mM MgCl₂, the purified fractions showed a glycosylation state of the CTB which was almost 100% (FIG. 27C).

These data show that CTB can be highly glycosylated by the disaccharide of N. meningitidis. Further, this glycosylated protein can be purified in high amounts. From each batch of 6 L of culture, between 5 and 15 mg of the protein of interest could be collected. Finally, the sugar structure was confirmed by MS analysis.

(c) Conclusions

The biosynthetic pathway for production of LLO's was reproduced using specific genes and enzymes from Neisseria, Campylobacter and other prokaryotic genes.

The mass spectrometry and liquid chromatography analyses of purified LLO demonstrated that such engineered glycan structures synthesized in engineered E. coli are identical to the said glycans identified in Neisseria.

In vivo glycosylation of several antigenic proteins (protein acceptor) from Gram positive and negative bacteria with the Neisseria oligosaccharides has been achieved generating new engineered prokaryotic cells (e.g. E. coli) by introducing genes encoding specific proteins to produce Neisseria undpp-oligosaccharides, protein acceptors and an oligosaccharyl transferase. Highly glycosylated proteins were purified from engineered E. coli to homogeneity using different liquid chromatography steps. The structures of the sugar were determined by mass spectrometry analysis of trypsin digested glycosylated proteins. The described methods have been used to produce milligram amounts of glycosylated protein per liter of culture.

The glycoconjugate produced with this approach can be used for preclinical assays to check the immunogenicity and serum bactericidal activity representative for the functionality of the protein conjugates as anti-neisserial conjugate vaccines.

### EQUIVALENTS

Although the invention is described in detail with reference to specific embodiments thereof, it will be understood that variations which were functionally equivalent were within the scope of this invention. Indeed, various modifications of the invention in addition to those shown and described herein will become apparent to those skilled in the art from the foregoing description and accompanying drawings. Such modifications were intended to fall within the scope of the appended claims. Those skilled in the art will recognize, or be able to ascertain using no more than routine experimentation, many equivalents to the specific embodiments of the invention described herein. Such equivalents were intended to be encompassed by the following claims.

All publications, patents and patent applications mentioned in this specification were herein incorporated by reference into the specification to the same extent as if each individual publication, patent or patent application was specifically and individually indicated to be incorporated herein by reference in their entireties.

1. A bioconjugate comprising (i) a carrier protein and a monosaccharide; (ii) a carrier protein and a disaccharide; or (iii) a carrier protein and a trisaccharide.

2. The bioconjugate of claim 1, wherein the monosaccharide, disaccharide, or trisaccharide is covalently bound to the Asn within a glycosylation site of the carrier protein wherein the glycosylation site comprises the amino acid sequence Asp/Glu-X-Asn-Z-Ser/Thr wherein X and Z may be any amino acid except Pro.

3. The bioconjugate of claim 2, wherein the glycosylation site has been recombinantly engineered and does not exist in the native carrier protein.

4. The bioconjugate of claim 2, wherein the carrier protein comprises 2, 3, 4, 5, 6, 7, 8, 9, or 10 glycosylation sites each having the amino acid sequence Asp/Glu-X-Asn-Z-Ser/Thr wherein X and Z may be any amino acid except Pro.

5. The bioconjugate of claim 1, wherein the monosaccharide is from N. meningitidis, the disaccharide is from N. meningitidis, or the trisaccharide is from N. meningitidis.

6. (canceled)

7. (canceled)

8. The bioconjugate of claim 1, wherein the carrier protein is selected from the group consisting of Exotoxin A of P. aeruginosa, CRM197, Diphtheria toxoid, tetanus toxoid, detoxified hemolysin A of S. aureus, clumping factor A, clumping factor B, E. coli FimH, E. coli FimHC, E. coli heat labile enterotoxin, detoxified variants of E. coli heat labile enterotoxin, Cholera toxin B subunit, cholera toxin, detoxified variants of cholera toxin, E. coli sat protein, the passenger domain of E. coli sat protein, C. jejuni AcrA, a C. jejuni natural glycoprotein, or an antigen of Neisseria meningitidis.

9. The bioconjugate of claim 8, wherein the antigen of N. meningitidis is from N. meningitidis group B; or wherein the antigen of N. meningitidis is pilin, NMB0088, nitrite reductase (AniA), heparin-binding antigen (NHBA), factor H binding protein (fHBP), adhesion, NadA, Ag473, or surface protein A (NapA).

10. (canceled)

11. (canceled)

12. The bioconjugate of claim 1 wherein the monosaccharide is DATDH or GATDH; wherein the disaccharide is Gal-DATDH, Gal(OAc)-DATDH, Gal-GATDH, Gal-GATDH, Gal(OAc)-GATDH, Gal-GlcNAc, Gal(OAc)-GlcNAc, Glc-DATDH, or Glc-GATDH or wherein the trisaccharide is Gal(OAc)-Gal-DATDH, Gal-Gal-DATDH, Gal(OAc)-Gal-GATDH, or Gal-Gal-GATDH.

13. (canceled)

14. (canceled)

15. An immunogenic composition comprising the bioconjugate of claim 1.

16. A prokaryotic host cell for generating a bioconjugate, wherein the prokaryotic host cell comprises:
a. a heterologous nucleotide sequence encoding a carrier protein comprising at least one glycosylation site comprising the amino acid sequence Asp/Glu-X-Asn-Z-Ser/Thr wherein X and Z may be any natural amino acid except Pro; and
b. a heterologous nucleotide sequence encoding an oligosaccaryltransferase;
wherein the prokaryotic host cell is recombinantly engineered to produce Und-PP-monosaccharide, Und-PP-disaccharide, or Und-PP-trisaccharide and wherein the oligosaccharyltransferase transfers the disaccharide or the trisaccharide to the Asn of the glycosylation site.

17. The prokaryotic host cell of claim 16, wherein the prokaryotic host organism is E. coli.

18. The prokaryotic host cell of claim 16, wherein the oligosaccaryltransferase is PglB of Campylobacter jejuni.

19. The prokaryotic host cell of claim 16, wherein the prokaryotic host cell further comprises a heterologous nucleotide sequence encoding a flippase; and/or a heterologous nucleotide sequence encoding a glycosyltransferase.

20. The prokaryotic host cell of claim 19, wherein the flippase is PglK of Campylobacter jejuni or PglF of Neisseria meningitidis.

21. (canceled)

22. The prokaryotic host cell of claim 19, wherein the glycosyltransferase is PglA of Neisseria meningitidis or RfpB of Shigella dysenteriae.

23. The prokaryotic host cell of claim 16, wherein said host cell produces Und-PP-DATDH; wherein said host cell produces Und-PP-GATDH; wherein said host cell produces and flips Und-PP-DATDH-Gal(OAc); or wherein said host cell produces and flips Und-PP-GATDH-Gal(OAc).

24. The prokaryotic host cell of claim 23, wherein said host cell produces Und-PP-DATDH or produces Und-PP-GATDH and wherein the host cell comprises N. meningitidis PglB, PglC, and PglD.

25. (canceled)

26. (canceled)

27. (canceled)

28. The prokaryotic host cell of claim 23, wherein said host cell produces and flips Und-PP-DATDH-Gal(OAc) and wherein said host cell comprises N. meningitidis PglB, PglC, PglD, PglF, PglA and PglI.

29. (canceled)

30. The prokaryotic host cell of claim 23, wherein said host cell produces and flips Und-PP-GATDH-Gal(OAc) and wherein said host cell comprises N. meningitidis PglB2, PglC, and PglD, PglF, PglA and PglI.

31. The prokaryotic host cell of claim 16, wherein the prokaryotic host cell comprises the pgl cluster of Campylobacter jejuni carrying a transposon mutation in galE.

32. The prokaryotic host cell of claim 16, wherein chloramphenicol, spectinomycin, and/or trimetoprim has been used as a drug selection marker for the generation of the prokaryotic host cell.

33. A method of generating a bioconjugate, wherein the method comprises:
a. culturing the prokaryotic host organism of claim 16; and
b. isolating the bioconjugate.

34. The method of claim 33, wherein the culturing step is performed in Terrific Broth (TB) medium and/or in the presence of MgCl₂.

35. (canceled)
